(19) United States
(12) Patent Application Publication
(10) Pub. No.: US 2025/0163072 A1
(43) Pub. Date: May 22, 2025

(54) MACROCYCLIC COMPOUNDS FOR THE TREATMENT OF CANCER

(71) Applicant: Hoffmann-La Roche Inc., Little Falls, NJ (US)

(72) Inventors: Jianguo CHEN, Shanghai (CN); Haixia LIU, Shanghai (CN); Hongxia QIU, Shanghai (CN); Hong SHEN, Shanghai (CN); Zhipeng YAN, Shanghai (CN); Beimeng YANG, Shanghai (CN); Xiangyu YAO, Shanghai (CN); Weixing ZHANG, Shanghai (CN); Dan ZHAO, Shanghai (CN); Wei ZHU, Shanghai (CN)

(73) Assignee: Hoffmann-La Roche Inc., Little Falls, NJ (US)

(21) Appl. No.: 18/687,262

(22) PCT Filed: Aug. 24, 2022
(86) PCT No.: PCT/EP2022/073542

§ 371 Date: Feb. 27, 2024

(30) Foreign Application Priority Data
Aug. 27, 2021 (WO) PCT/CN2021/115095
Dec. 9, 2021 (WO) PCT/CN2021/136793
Mar. 14, 2022 (WO) PCT/CN2022/080635
Jun. 13, 2022 (WO) PCT/CN2022/098430

(51) Int. Cl. C07D 491/16; A61K 31/5025; A61K 31/5377; C07D 513/22; C07D 519/00

(52) U.S. Cl. CPC C07D 491/16; A61K 31/5025; A61K 31/5377; C07D 513/22; C07D 519/00

(57) ABSTRACT

The present invention relates to compounds of formula (I), wherein R¹ to R⁵ and A¹ to A³ are as described herein, and their pharmaceutically acceptable salt thereof, and compositions including the compounds and methods of using the compounds.

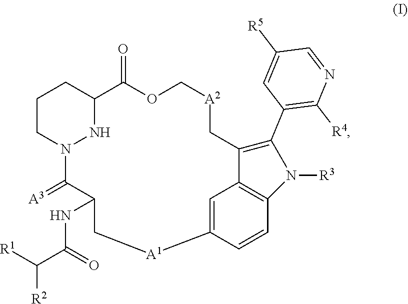

The present invention relates to organic compounds useful for therapy and/or prophylaxis in a mammal, and in particular to inhibition of KRAS G12C useful for treating cancers.

### FIELD OF THE INVENTION

RAS is one of the most well-known proto-oncogenes. Approximately 30% of human cancers contain mutations in three most notable members, KRAS, HRAS, and NRAS, making them the most prevalent oncogenic drivers. KRAS mutations are generally associated with poor prognosis especially in colorectal cancer, pancreatic cancer, lung cancers. As the most frequently mutated RAS isoform, KRAS has been intensively studied in the past years. Among the most commonly occurring KRAS alleles (including G12D, G12V, G12C, G13D, G12R, G12A, G12S, Q61H, etc), G12C, G12D, G12V represent more than half of all K-RAS-driven cancers across colorectal cancer (CRC), pancreatic ductal adenocarcinoma (PDAC), lung adenocarcinoma (LUAD). Of note, KRAS wild-type amplifications are also found in around 7% of all KRAS-altered cancers (ovarian, esophagogastric, uterine), ranking among the top alterations.

All RAS proteins belong to a protein family of small GTPases that hydrolyze GTP to GDP. KRAS is structurally divided into an effector binding lobe followed by the allosteric lobe and a carboxy-terminal region that is responsible for membrane anchoring. The effector lobe comprises the P-loop, switch I, and switch II regions. The switch I/II loops play a critical role in KRAS downstream signaling through mediating protein-protein interactions with effector proteins that include RAF in the mitogen-activated protein kinase (MAPK) pathway or PI3K in the phosphatidylinositol 3-kinase (PI3K)/protein kinase B (AKT) pathway.

KRAS protein switches between an inactive to an active form via binding to GTP and GDP, respectively. Under physiological conditions, the transition between these two states is regulated by guanine nucleotide exchange factors (GEFs), such as Son Of Sevenless Homolog 1 (SOS1), or GTPase-activating proteins (GAPs) that involve catalyzing the exchange of GDP for GTP, potentiating intrinsic GTPase activity or accelerating RAS-mediated GTP hydrolysis. In response to extracellular stimuli, the inactive RAS-GDP is converted to active RAS-GTP which directly binds to RAF RAS binding domains (RAF^{RBD}), recruiting RAF kinase family from cytoplasm to membranes, where they dimerize and become active. The activated RAF subsequently carries out a chain of phosphorylation reactions to its downstream Mitogen-activated protein kinase (MEK) and extracellular signal-regulated kinase (ERK), and propagates the growth signal. Of the RAF family of protein kinases (three known isoforms ARAF, BRAF, CRAF/RAF1), BRAF is most frequently mutated and remains the most potent activator of MEK.

Despite that individual RAS and RAF family members revealed distinct binding preferences, all RAFs possess the conserved RBD for forward transmission of MAPK signaling, frequently used for characterize KRAS inhibition (e.g. KRAS-BRAF^{RBD} herein). For KRAS, mutations at positions 12, 13, 61, and 146 lead to a shift toward the active KRAS form through impairing nucleotide hydrolysis or activating nucleotide exchange, leading to hyper-activation of the MAPK pathway that results in tumorigenesis.

Despite its well-recognized importance in cancer malignancy, continuous efforts in the past failed to develop approved therapies for KRAS mutant cancer until recently, the first selective drug AMG510 has fast approval as second line treatment in KRAS G12C driven non-small cell lung cancer (NSCLC). Nevertheless, the clinical acquired resistance to KRAS G12C inhibitors emerge rigorously with disease progresses after around 6 month of treatment. All of the mutations converge to reactivate RAS-MAPK signaling, with secondary RAS mutants at oncogenic hotspots (e.g. G12/G13/Q61) and within the switch II pocket (e.g. H95, R68, and Y96) have been observed; moreover, over 85% of all KRAS-mutated or wild-type amplified driven cancers still lack novel agents. Altogether, both the myriad of escape mechanism and various oncogenic alleles, highlight the urgent medical need for additional KRAS therapies. As such, we invented oral compounds that target and inhibit KRAS alleles for the treatment of KRAS mutant driven cancers.

### SUMMARY OF THE INVENTION

The present invention relates to novel compounds of formula (I),

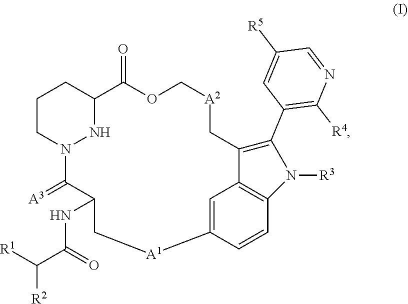

wherein
R¹ is

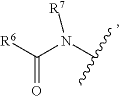

or 6-oxo-2,7-diazaspiro[4.5]decan-7-yl substituted by (dihaloC₁₋₆alkyl)carbonyl;
   wherein R⁶ is azetidinyl substituted by C₂₋₆alkynyl,
      cycloalkyl substituted by formyl, C₂₋₆alkynyl, pyridinylC₂₋₆alkynyl or [(C₁₋₆alkyl)₂(oxo)-λ⁶-sulfanylidene]C₁₋₆alkylcarbonyl,
      piperidinyl once or twice substituted by substituents independently selected from halogen, (dihaloC₁₋₆alkyl)carbonyl and C₂₋₆alkynyl, or
      pyrrolidinyl substituted by (C₁₋₆alkylcarbonyl)carbonyl, (dihaloC₁₋₆alkyl)carbonyl, C₂₋₆alkynyl, cyanoC₁₋₆alkyl, cycloalkylcarbonyl, or triazolylC₂₋₆alkenylcarbonyl;
   R⁷ is C₁₋₆alkyl;
R² is C₁₋₆alkyl;
R³ is C₁₋₆alkyl or haloC₁₋₆alkyl;
R⁴ is C₁₋₆alkoxyC₁₋₆alkyl;
R⁵ is H, morpholinyl, (haloC₁₋₆alkyl)piperazinyl or C₁₋₆alkylpiperazinyl;
A¹ is thiazolylene or phenylene, said phenylene being substituted by hydroxy;
A² is C₁₋₆alkylene;
A³ is O;
or a pharmaceutically acceptable salt thereof.

The invention also relates to their manufacture, medicaments based on a compound in accordance with the invention and their production as well as the use of compounds of formula (I) or (Ia) thereof as inhibitor of KRAS.

The compounds of formula (I) or (Ia) show good KRAS inhibition for G12C, G12D and G12V. In another embodiment, the compounds of this invention showed superior cancer cell inhibition. In addition, the compounds of formula (I) or (Ia) also show good or improved cytotoxicity, solubility profiles. Furthermore, the compound of current invention addressed GSH toxicity issue comparing with the reference compounds (see Example 27).

### BRIEF DESCRIPTION OF THE FIGURE

FIG. 1. X-ray crystallographic analysis of Intermediate A

### DETAILED DESCRIPTION OF THE INVENTION

### Definitions

The term “C₁₋₆alkyl” denotes a saturated, linear or branched chain alkyl group containing 1 to 6, particularly 1 to 4 carbon atoms, for example methyl, ethyl, n-propyl, isopropyl, n-butyl, isobutyl, tert-butyl and the like. Particular “C₁₋₆alkyl” groups are methyl, ethyl and n-propyl.

The term “C₁₋₆alkylene” denotes a linear or branched saturated divalent hydrocarbon group of 1 to 6 carbon atoms or a divalent branched saturated divalent hydrocarbon group of 3 to 6 carbon atoms. Examples of C₁₋₆alkylene groups include methylene, ethylene, propylene, 2-methylpropylene, butylene, 2-ethylbutylene, pentylene, hexylene.

The term “C₂₋₆alkenyl” denotes a monovalent linear or branched hydrocarbon group of 2 to 6 carbon atoms with at least one double bond. In particular embodiments, alkenyl has 2 to 4 carbon atoms with at least one double bond. Examples of C₂₋₆alkenyl include ethenyl (or vinyl), propenyl, prop-2-enyl, isopropenyl, n-butenyl, and iso-butenyl.

The term “C₂₋₆alkynyl” denotes a monovalent linear or branched saturated hydrocarbon group of 2 to 6 carbon atoms comprising one, two or three triple bonds. In particular embodiments alkynyl has from 2 to 4 carbon atoms comprising one or two triple bonds. Examples of C₂₋₆alkynyl include ethynyl, propynyl, prop-2-ynyl, isopropynyl, and n-butynyl.

The term “C₃₋₈alkadienyl” denotes hydrocarbons of 3 to 8 carbon atoms containing two double bonds between carbon atoms. These two double bonds can be present in the molecule at different positions, connected to the same carbon atom (cumulated, C═C═C), or separated from each other by a single a bond (conjugated, C═C—C═C). Examples of “C₃₋₈alkadienyl” include butadienyl.

The term “cycloalkyl” denotes a monovalent saturated monocyclic or bicyclic hydrocarbon group of 3 to 10 ring carbon atoms. In particular embodiments, cycloalkyl denotes a monovalent saturated monocyclic hydrocarbon group of 3 to 8 ring carbon atoms. Bicyclic means consisting of two saturated carbocycles having one or more carbon atoms in common. Examples for monocyclic cycloalkyl are cyclopropyl, cyclobutyl, cyclopentyl, cyclohexyl or cycloheptyl. Examples for bicyclic cycloalkyl are bicyclo[1.1.0]butyl, bicyclo[2.2.1]heptanyl, or bicyclo[2.2.2]octanyl.

The term “halogen” and “halo” are used interchangeably herein and denote fluoro, chloro, bromo, or iodo.

The term “dihaloC₁₋₆alkyl” denotes a C₁₋₆alkyl group wherein two of the hydrogen atoms of the C₁₋₆alkyl group have been replaced by same or different halogen atoms. Examples of dihaloC₁₋₆alkyl include, difluoro- or chloro(fluoro)-methyl, -ethyl or -propyl, for example difluoropropyl, difluoromethyl, difluoroethyl or chloro(fluoro)methyl.

The term “haloC₁₋₆alkyl” denotes a C₁₋₆alkyl group wherein at least one of the hydrogen atoms of the C₁₋₆alkyl group have been replaced by same or different halogen atoms. Examples of haloC₁₋₆alkyl include fluoro, difluoro- or chloro(fluoro)-methyl, -ethyl or -propyl, for example fluoromethyl, difluoropropyl, difluoromethyl, difluoroethyl, chloro(fluoro)methyl, trifluoroethyl, or trifluoromethyl.

The term “phenylene” denotes a divalent phenyl group.

The term “thiazolylene” denotes a divalent thiazolyl group.

The term “oxo” denotes a divalent oxygen atom ═O.

The term “sulfanyl” denotes a —S— group.

The term “heterocyclyl” denotes a monovalent saturated or partly unsaturated mono- or bicyclic ring system of 3 to 9 ring atoms, comprising 1, 2, or 3 ring heteroatoms selected from N, O and S, the remaining ring atoms being carbon. In particular embodiments, heterocyclyl is a monovalent saturated monocyclic ring system of 4 to 7 ring atoms, comprising 1, 2, or 3 ring heteroatoms selected from N, O and S, the remaining ring atoms being carbon. Examples for monocyclic saturated heterocyclyl are aziridinyl, oxiranyl, azetidinyl, oxetanyl, pyrrolidinyl, tetrahydrofuranyl, tetrahydro-thienyl, pyrazolidinyl, imidazolidinyl, oxazolidinyl, isoxazolidinyl, thiazolidinyl, piperidinyl, tetrahydropyranyl, tetrahydrothiopyranyl, piperazinyl, morpholinyl, thiomorpholinyl, 1,1-dioxo-thiomorpholin-4-yl, azepanyl, diazepanyl, homopiperazinyl, or oxazepanyl. Examples for bicyclic saturated heterocyclyl are 8-aza-bicyclo[3.2.1]octyl, quinuclidinyl, 8-oxa-3-aza-bicyclo[3.2.1]octyl, 9-aza-bicyclo[3.3.1]nonyl, 3-oxa-9-aza-bicyclo[3.3.1]nonyl, or 3-thia-9-aza-bicyclo[3.3.1]nonyl. Examples for partly unsaturated heterocyclyl are dihydrofuryl, imidazolinyl, dihydro-oxazolyl, tetrahydro-pyridinyl, or dihydropyranyl. The term “heterocyclylene” denotes a divalent heterocyclyl group.

The term “protecting group” denotes the group which selectively blocks a reactive site in a multifunctional compound such that a chemical reaction can be carried out selectively at another unprotected reactive site in the meaning conventionally associated with it in synthetic chemistry. Protecting groups can be removed at the appropriate point. Exemplary protecting groups are amino-protecting groups, carboxy-protecting groups or hydroxy-protecting groups.

The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

The term “A pharmaceutically active metabolite” denotes a pharmacologically active product produced through metabolism in the body of a specified compound or salt thereof. After entry into the body, most drugs are substrates for chemical reactions that may change their physical properties and biologic effects. These metabolic conversions, which usually affect the polarity of the compounds of the invention, alter the way in which drugs are distributed in and excreted from the body. However, in some cases, metabolism of a drug is required for therapeutic effect.

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

### Inhibitor of KRAS

The present invention relates to (i) a compound of formula (I),

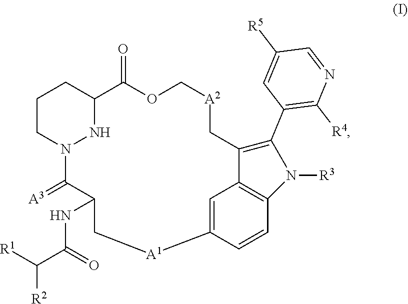

wherein
R¹ is

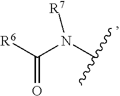

1-oxo-2,7-diazaspiro[4.4]nonan-2-yl substituted by (dihaloC₁₋₆alkyl)carbonyl or 6-oxo-2,7-diazaspiro[4.5]decan-7-yl substituted by (dihaloC₁₋₆alkyl)carbonyl;
   wherein R⁶ is azetidinyl substituted by C₂₋₆alkynyl,
      cycloalkyl substituted by formyl, C₂₋₆alkynyl, pyridinylC₂₋₆alkynyl or [(C₁₋₆alkyl)₂(oxo)-λ⁶-sulfanylidene]C₁₋₆alkylcarbonyl,
      piperidinyl once or twice substituted by substituents independently selected from halogen, (dihaloC₁₋₆alkyl)carbonyl and C₂₋₆alkynyl, or
      pyrrolidinyl substituted by (C₁₋₆alkylcarbonyl)carbonyl, (dihaloC₁₋₆alkyl)carbonyl, C₂₋₆alkynyl, cyanoC₁₋₆alkyl, cycloalkylcarbonyl, or triazolylC₂₋₆alkenylcarbonyl;
   R⁷ is C₁₋₆alkyl;
R² is C₁₋₆alkyl;
R³ is C₁₋₆alkyl or haloC₁₋₆alkyl;
R⁴ is C₁₋₆alkoxyC₁₋₆alkyl;
R⁵ is H, morpholinyl, (haloC₁₋₆alkyl)piperazinyl or C₁₋₆alkylpiperazinyl;
A¹ is thiazolylene or phenylene, said phenylene being substituted by hydroxy;
A² is C₁₋₆alkylene;
A³ is O;
or a pharmaceutically acceptable salt thereof.

Another embodiment of present invention is (ii) a compound of formula (Ia),

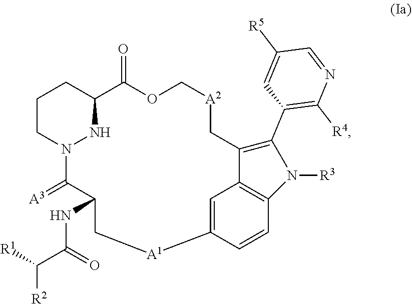

wherein
R¹ is

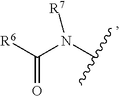

1-oxo-2,7-diazaspiro[4.4]nonan-2-yl substituted by (dihaloC₁₋₆alkyl)carbonyl or 6-oxo-2,7-diazaspiro[4.5]decan-7-yl substituted by (dihaloC₁₋₆alkyl)carbonyl;
   wherein R⁶ is azetidinyl substituted by C₂₋₆alkynyl,
      cycloalkyl substituted by formyl, C₂₋₆alkynyl, pyridinylC₂₋₆alkynyl or [(C₁₋₆alkyl)₂(oxo)-λ⁶-sulfanylidene]C₁₋₆alkylcarbonyl,
      piperidinyl once or twice substituted by substituents independently selected from halogen, (dihaloC₁₋₆alkyl)carbonyl and C₂₋₆alkynyl, or
      pyrrolidinyl substituted by (C₁₋₆alkylcarbonyl)carbonyl, (dihaloC₁₋₆alkyl)carbonyl, C₂₋₆alkynyl, cyanoC₁₋₆alkyl, cycloalkylcarbonyl, or triazolylC₂₋₆alkenylcarbonyl;
   R⁷ is C₁₋₆alkyl;
R² is C₁₋₆alkyl;
R³ is C₁₋₆alkyl or haloC₁₋₆alkyl;
R⁴ is C₁₋₆alkoxyC₁₋₆alkyl;
R⁵ is H, morpholinyl, (haloC₁₋₆alkyl)piperazinyl or C₁₋₆alkylpiperazinyl;
A¹ is thiazolylene or phenylene, said phenylene being substituted by hydroxy;
A² is C₁₋₆alkylene;
A³ is O;
or a pharmaceutically acceptable salt thereof.

A further embodiment of present invention is (iii) a compound of formula (I) or (Ia) according to (i) or (ii), or a pharmaceutically acceptable salt thereof, wherein R¹ is

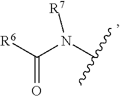

1-oxo-2,7-diazaspiro[4.4]nonan-2-yl substituted by (dihaloC₁₋₆alkyl)carbonyl or 6-oxo-2,7-diazaspiro[4.5]decan-7-yl substituted by (dihaloC₁₋₆alkyl)carbonyl; wherein R⁶ is pyrrolidinyl substituted by (dihaloC₁₋₆alkyl)carbonyl; R⁷ is C₁₋₆alkyl.

A further embodiment of present invention is (iv) a compound of formula (I) or (Ia), according to any one of (i) to (iii), or a pharmaceutically acceptable salt thereof, wherein R¹ is

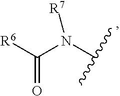

1-oxo-2,7-diazaspiro[4.4]nonan-2-yl substituted by chloro(fluoro)acetyl or 6-oxo-2,7-diazaspiro[4.5]decan-7-yl substituted by chloro(fluoro)acetyl; wherein R⁶ is pyrrolidinyl substituted by chloro(fluoro)acetyl; R⁷ is methyl.

A further embodiment of present invention is (v) a compound of formula (I) or (Ia) according to any one of (i) to (iv), wherein R¹ is

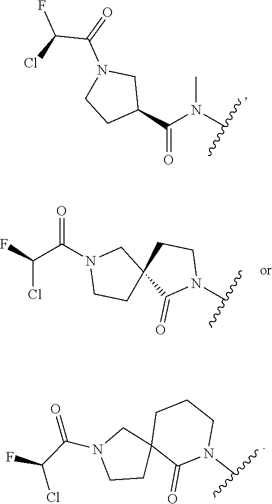 [SMILES: *N(C)C(=O)[C@H]1CCN(C(=O)[C@H](F)Cl)C1 ; *N1CC[C@]2(CCN(C(=O)[C@H](F)Cl)C2)C1=O ; *N1CCCC2(CCN(C(=O)[C@H](F)Cl)C2)C1=O]

A further embodiment of present invention is (vi) a compound of formula (I) or (Ia), or a pharmaceutically acceptable salt thereof, according to any one of (i) to (v), wherein R² is isopropyl.

A further embodiment of present invention is (vii) a compound of formula (I) or (Ia), or a pharmaceutically acceptable salt thereof, according to any one of (i) to (vi), wherein R³ is ethyl or trifluoroethyl.

A further embodiment of present invention is (viii) a compound of formula (I) or (Ia), or a pharmaceutically acceptable salt thereof, according to any one of (i) to (vii), wherein R⁴ is methoxyethyl.

A further embodiment of present invention is (ix) a compound of formula (I) or (Ta), or a pharmaceutically acceptable salt thereof, according to any one of (i) to (viii), wherein R⁴ is

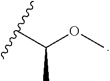 [SMILES: *[C@H](C)OC]

A further embodiment of present invention is (x) a compound of formula (I) or (Ta), or a pharmaceutically acceptable salt thereof, according to any one of (i) to (ix), wherein R⁵ is morpholinyl or methylpiperazinyl.

A further embodiment of present invention is (xi) a compound of formula (I) or (Ia), or a pharmaceutically acceptable salt thereof, according to any one of (i) to (x), wherein A¹ is

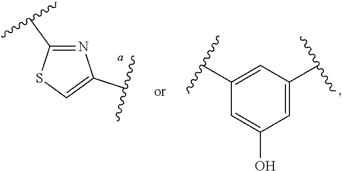 [SMILES: *c1csc(*)n1 ; *c1cc(*)cc(O)c1]

wherein bond “a” connects to indole ring.

A further embodiment of present invention is (xii) a compound of formula (I) or (Ia), or a pharmaceutically acceptable salt thereof, according to any one of (i) to (xi), wherein A² is dimethylmethylene.

A further embodiment of present invention is (xiii) a compound of formula (I) or (Ia), according to any one of (i) to (xii), wherein
R¹ is

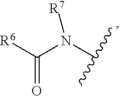

1-oxo-2,7-diazaspiro[4.4]nonan-2-yl substituted by (dihaloC₁₋₆alkyl)carbonyl or 6-oxo-2,7-diazaspiro[4.5]decan-7-yl substituted by (dihaloC₁₋₆alkyl)carbonyl;
   wherein R⁶ is pyrrolidinyl substituted by (dihaloC₁₋₆alkyl)carbonyl; R⁷ is C₁₋₆alkyl;
R² is C₁₋₆alkyl;
R³ is C₁₋₆alkyl or haloC₁₋₆alkyl;
R⁴ is C₁₋₆alkoxyC₁₋₆alkyl;
R⁵ is morpholinyl or C₁₋₆alkylpiperazinyl;
A¹ is thiazolylene or phenylene, said phenylene being substituted by hydroxy;
A² is C₁₋₆alkylene;
A³ is O;
or a pharmaceutically acceptable salt thereof.

A further embodiment of present invention is (xiv) a compound of formula (I) or (Ia), according to any one of (i) to (xiii), wherein
R¹ is

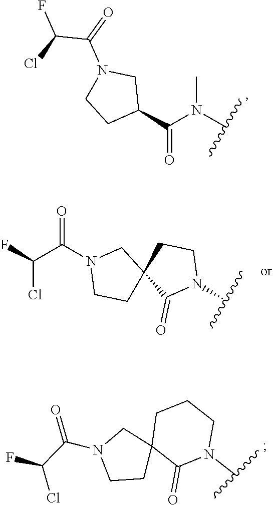

R² is isopropyl;
R³ is ethyl or trifluoroethyl;
R⁴ is

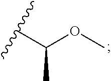 [SMILES: *[C@H](C)OC]

R⁵ is morpholinyl or methylpiperazinyl;
A¹ is or

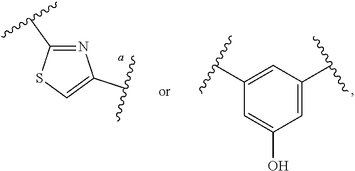 [SMILES: *c1csc(*)n1 ; *c1cc(*)cc(O)c1]

wherein bond “a” connects to indole ring;
A² is dimethylmethylene;
A³ is O;
or a pharmaceutically acceptable salt thereof.

The present invention relates to (i′) a compound of formula (I),

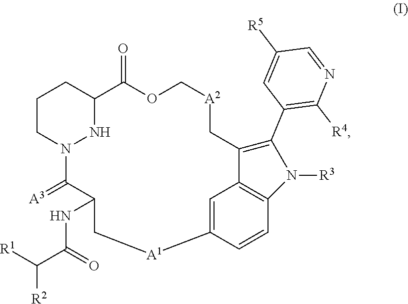

wherein
R¹ is

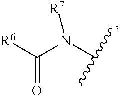

or 6-oxo-2,7-diazaspiro[4.5]decan-7-yl substituted by (dihaloC₁₋₆alkyl)carbonyl;
   wherein R⁶ is azetidinyl substituted by C₂₋₆alkynyl,
      cycloalkyl substituted by formyl, C₂₋₆alkynyl, pyridinylC₂₋₆alkynyl or [(C₁₋₆alkyl)₂(oxo)-λ⁶-sulfanylidene]C₁₋₆alkylcarbonyl,
      piperidinyl once or twice substituted by substituents independently selected from halogen, (dihaloC₁₋₆alkyl)carbonyl and C₂₋₆alkynyl, or
      pyrrolidinyl substituted by (C₁₋₆alkylcarbonyl)carbonyl, (dihaloC₁₋₆alkyl)carbonyl, C₂₋₆alkynyl, cyanoC₁₋₆alkyl, cycloalkylcarbonyl, or triazolylC₂₋₆alkenylcarbonyl;
   R⁷ is C₁₋₆alkyl;
R² is C₁₋₆alkyl;
R³ is C₁₋₆alkyl or haloC₁₋₆alkyl;
R⁴ is C₁₋₆alkoxyC₁₋₆alkyl;
R⁵ is H, morpholinyl, (haloC₁₋₆alkyl)piperazinyl or C₁₋₆alkylpiperazinyl;
A¹ is thiazolylene or phenylene, said phenylene being substituted by hydroxy;
A² is C₁₋₆alkylene;
A³ is O;
or a pharmaceutically acceptable salt thereof.

Another embodiment of present invention is (ii′) a compound of formula (Ia),

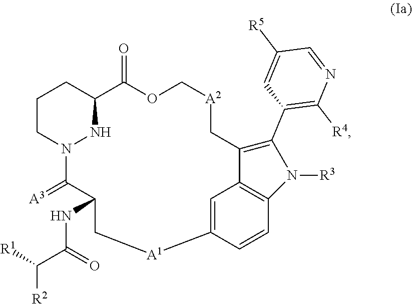

wherein
R¹ is

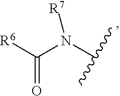

or 6-oxo-2,7-diazaspiro[4.5]decan-7-yl substituted by (dihaloC₁₋₆alkyl)carbonyl;
   wherein R⁶ is azetidinyl substituted by C₂₋₆alkynyl,
      cycloalkyl substituted by formyl, C₂₋₆alkynyl, pyridinylC₂₋₆alkynyl or [(C₁₋₆alkyl)₂(oxo)-λ⁶-sulfanylidene]C₁₋₆alkylcarbonyl,
      piperidinyl once or twice substituted by substituents independently selected from halogen, (dihaloC₁₋₆alkyl)carbonyl and C₂₋₆alkynyl, or
      pyrrolidinyl substituted by (C₁₋₆alkylcarbonyl)carbonyl, (dihaloC₁₋₆alkyl)carbonyl, C₂₋₆alkynyl, cyanoC₁₋₆alkyl, cycloalkylcarbonyl, or triazolylC₂₋₆alkenylcarbonyl;
   R⁷ is C₁₋₆alkyl;
R² is C₁₋₆alkyl;
R³ is C₁₋₆alkyl or haloC₁₋₆alkyl;
R⁴ is C₁₋₆alkoxyC₁₋₆alkyl;
R⁵ is H, morpholinyl, (haloC₁₋₆alkyl)piperazinyl or C₁₋₆alkylpiperazinyl;
A¹ is thiazolylene or phenylene, said phenylene being substituted by hydroxy;
A² is C₁₋₆alkylene;
A³ is O;
or a pharmaceutically acceptable salt thereof.

A further embodiment of present invention is (iii′) a compound of formula (I) or (Ia) according to (i′) or (ii′), or a pharmaceutically acceptable salt thereof, wherein R¹ is

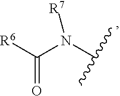

or 6-oxo-2,7-diazaspiro[4.5]decan-7-yl substituted by (dihaloC₁₋₆alkyl)carbonyl; wherein R⁶ is pyrrolidinyl substituted by (dihaloC₁₋₆alkyl)carbonyl; R⁷ is C₁₋₆alkyl.

A further embodiment of present invention is (iv′) a compound of formula (I) or (Ia), according to any one of (i′) to (iii′), or a pharmaceutically acceptable salt thereof, wherein R¹ is

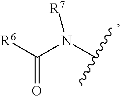

or 6-oxo-2,7-diazaspiro[4.5]decan-7-yl substituted by chloro(fluoro)acetyl; wherein R⁶ is pyrrolidinyl substituted by chloro(fluoro)acetyl; R⁷ is methyl.

A further embodiment of present invention is (v′) a compound of formula (I) or (Ia) according to any one of (i′) to (iv′), wherein R¹ is

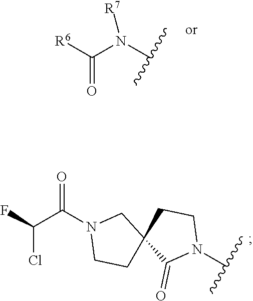

wherein R⁶ is

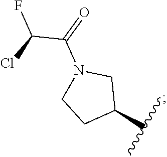 [SMILES: *[C@H]1CCN(C(=O)[C@H](F)Cl)C1]

R⁷ is methyl.

A further embodiment of present invention is (vi′) a compound of formula (I) or (Ta), or a pharmaceutically acceptable salt thereof, according to any one of (i′) to (v′), wherein R² is isopropyl.

A further embodiment of present invention is (vii′) a compound of formula (I) or (Ia), or a pharmaceutically acceptable salt thereof, according to any one of (i) to (vi), wherein R³ is ethyl or trifluoroethyl.

A further embodiment of present invention is (viii′) a compound of formula (I) or (Ta), or a pharmaceutically acceptable salt thereof, according to any one of (i) to (vii), wherein R⁴ is methoxyethyl.

A further embodiment of present invention is (ix′) a compound of formula (I) or (Ta), or a pharmaceutically acceptable salt thereof, according to any one of (i′) to (viii′), wherein R⁴ is

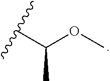 [SMILES: *[C@H](C)OC]

A further embodiment of present invention is (x′) a compound of formula (I) or (Ta), or a pharmaceutically acceptable salt thereof, according to any one of (i′) to (ix′), wherein R⁵ is morpholinyl or methylpiperazinyl.

A further embodiment of present invention is (xi′) a compound of formula (I) or (Ta), or a pharmaceutically acceptable salt thereof, according to any one of (i′) to (x′), wherein A¹ is

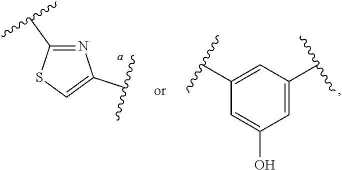 [SMILES: *c1csc(*)n1 ; *c1cc(*)cc(O)c1]

wherein bond “a” connects to indole ring.

A further embodiment of present invention is (xii′) a compound of formula (I) or (Ia), or a pharmaceutically acceptable salt thereof, according to any one of (i′) to (xi′), wherein A² is dimethylmethylene.

A further embodiment of present invention is (xiii′) a compound of formula (I) or (Ia), according to any one of (i′) to (xii′), wherein
R¹ is

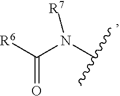

or 6-oxo-2,7-diazaspiro[4.5]decan-7-yl substituted by (dihaloC₁₋₆alkyl)carbonyl;
   wherein R⁶ is pyrrolidinyl substituted by (dihaloC₁₋₆alkyl)carbonyl; R⁷ is C₁₋₆alkyl;
R² is C₁₋₆alkyl;
R³ is C₁₋₆alkyl or haloC₁₋₆alkyl;
R⁴ is C₁₋₆alkoxyC₁₋₆alkyl;
R⁵ is morpholinyl or C₁₋₆alkylpiperazinyl;
A¹ is thiazolylene or phenylene, said phenylene being substituted by hydroxy;
A² is C₁₋₆alkylene;
A³ is O;
or a pharmaceutically acceptable salt thereof.

A further embodiment of present invention is (xiv′) a compound of formula (I) or (Ia), according to any one of (i′) to (xiii′), wherein
R¹ is

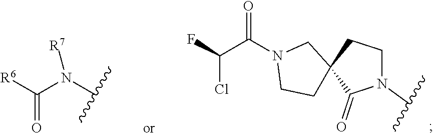

wherein R⁶ is

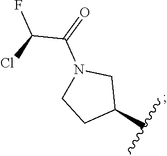 [SMILES: *[C@H]1CCN(C(=O)[C@H](F)Cl)C1]

R⁷ is methyl;
R² is isopropyl;
R³ is ethyl or trifluoroethyl;
R⁴ is

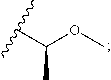 [SMILES: *[C@H](C)OC]

R⁵ is morpholinyl or methylpiperazinyl;
A¹ is

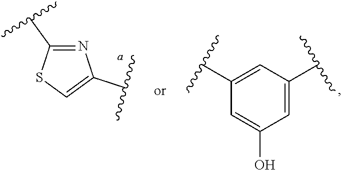 [SMILES: *c1csc(*)n1 ; *c1cc(*)cc(O)c1]

wherein bond “a” connects to indole ring;
A² is dimethylmethylene;
A³ is O;
or a pharmaceutically acceptable salt thereof.

The present invention relates to (i″) a compound of formula (I),

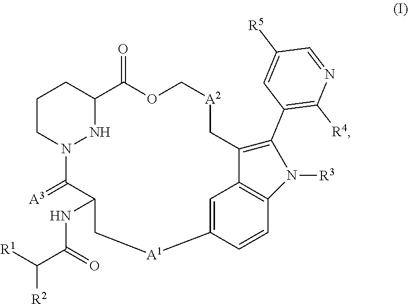

wherein
R¹ is

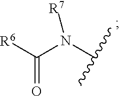

wherein R⁶ is azetidinyl substituted by C₂₋₆alkynyl,
   cycloalkyl substituted by formyl, C₂₋₆alkynyl, pyridinylC₂₋₆alkynyl or [(C₁₋₆alkyl)₂(oxo)-λ⁶-sulfanylidene]C₁₋₆alkylcarbonyl,
   piperidinyl once or twice substituted by substituents independently selected from halogen and C₂₋₆alkynyl, or
   pyrrolidinyl substituted by (C₁₋₆alkylcarbonyl)carbonyl, (dihaloC₁₋₆alkyl)carbonyl, C₂₋₆alkynyl, cyanoC₁₋₆alkyl, cycloalkylcarbonyl, or triazolylC₂₋₆alkenylcarbonyl;
R⁷ is C₁₋₆alkyl;
R² is C₁₋₆alkyl;
R³ is C₁₋₆alkyl;
R⁴ is C₁₋₆alkoxyC₁₋₆alkyl;
R⁵ is H or C₁₋₆alkylpiperazinyl;
A¹ is thiazolylene or phenylene, said phenylene being substituted by hydroxy;
A² is C₁₋₆alkylene;
A³ is O;
or a pharmaceutically acceptable salt thereof.

Another embodiment of present invention is (ii″) a compound of formula (Ia),

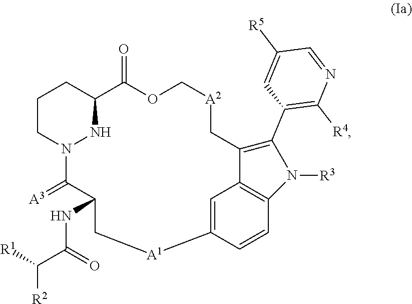

wherein
R¹ is

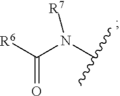

wherein R⁶ is azetidinyl substituted by C₂₋₆alkynyl,
   cycloalkyl substituted by formyl, C₂₋₆alkynyl, pyridinylC₂₋₆alkynyl or [(C₁₋₆alkyl)₂(oxo)-λ⁶-sulfanylidene]C₁₋₆alkylcarbonyl,
   piperidinyl once or twice substituted by substituents independently selected from halogen and C₂₋₆alkynyl, or
   pyrrolidinyl substituted by (C₁₋₆alkylcarbonyl)carbonyl, (dihaloC₁₋₆alkyl)carbonyl, C₂₋₆alkynyl, cyanoC₁₋₆alkyl, cycloalkylcarbonyl, or triazolylC₂₋₆alkenylcarbonyl;
R⁷ is C₁₋₆alkyl;
R² is C₁₋₆alkyl;
R³ is C₁₋₆alkyl;
R⁴ is C₁₋₆alkoxyC₁₋₆alkyl;
R⁵ is H or C₁₋₆alkylpiperazinyl;
A¹ is thiazolylene or phenylene, said phenylene being substituted by hydroxy;
A² is C₁₋₆alkylene;
A³ is O;
or a pharmaceutically acceptable salt thereof.

A further embodiment of present invention is (iii″) a compound of formula (I) or (Ia) according to (i″) or (ii″), or a pharmaceutically acceptable salt thereof, wherein R¹ is

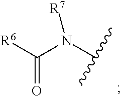

wherein R⁶ is pyrrolidinyl substituted by (dihaloC₁₋₆alkyl)carbonyl; R⁷ is C₁₋₆alkyl.

A further embodiment of present invention is (iv″) a compound of formula (I) or (Ia), according to any one of (i″) to (iii″), or a pharmaceutically acceptable salt thereof, wherein R¹ is

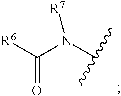

wherein R⁶ is pyrrolidinyl substituted by chloro(fluoro)acetyl; R⁷ is methyl.

A further embodiment of present invention is (v″) a compound of formula (I) or (Ia) according to any one of (i″) to (iv″), wherein
R¹ is

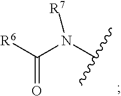

wherein R⁶ is

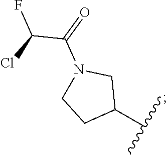 [SMILES: *C1CCN(C(=O)[C@H](F)Cl)C1]

R⁷ is methyl.

A further embodiment of present invention is (vi″) a compound of formula (I) or (Ia), or a pharmaceutically acceptable salt thereof, according to any one of (i″) to (v″), wherein R² is isopropyl.

A further embodiment of present invention is (vii″) a compound of formula (I) or (Ia), or a pharmaceutically acceptable salt thereof, according to any one of (i″) to (vi″), wherein R³ is ethyl.

A further embodiment of present invention is (viii″) a compound of formula (I) or (Ia), or a pharmaceutically acceptable salt thereof, according to any one of (i″) to (vii′″), wherein R⁴ is methoxyethyl.

A further embodiment of present invention is (ix″) a compound of formula (I) or (Ia), or a pharmaceutically acceptable salt thereof, according to any one of (i′) to (viii′), wherein R⁴ is

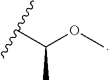 [SMILES: *[C@H](C)OC]

A further embodiment of present invention is (x″) a compound of formula (I) or (Ta), or a pharmaceutically acceptable salt thereof, according to any one of (i″) to (ix″), wherein R⁵ is H or methylpiperazinyl.

A further embodiment of present invention is (xi″) a compound of formula (I) or (Ta), or a pharmaceutically acceptable salt thereof, according to any one of (i″) to (x″), wherein A¹ is

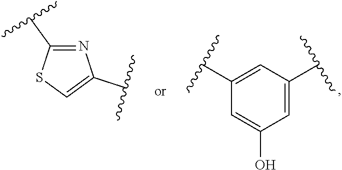 [SMILES: *c1csc(*)n1 ; *c1cc(*)cc(O)c1]

A further embodiment of present invention is (xii″) a compound of formula (I) or (Ta), or a pharmaceutically acceptable salt thereof, according to any one of (i″) to (xi″), wherein A² is dimethylmethylene.

A further embodiment of present invention is (xiii″) a compound of formula (I) or (Ta), according to any one of (i″) to (xii″), wherein
R¹ is

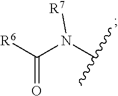

wherein R⁶ is pyrrolidinyl substituted by (dihaloC₁₋₆alkyl)carbonyl; R⁷ is C₁₋₆alkyl;
R² is C₁₋₆alkyl;
R³ is C₁₋₆alkyl;
R⁴ is C₁₋₆alkoxyC₁₋₆alkyl;
R⁵ is H or C₁₋₆alkylpiperazinyl;
A¹ is thiazolylene or phenylene, said phenylene being substituted by hydroxy;
A² is C₁₋₆alkylene;
A³ is O;
or a pharmaceutically acceptable salt thereof.

A further embodiment of present invention is (xiv″) a compound of formula (I) or (Ia), according to any one of (i″) to (xiii″), wherein
R¹ is

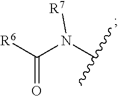

wherein R⁶ is

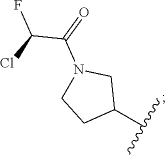 [SMILES: *C1CCN(C(=O)[C@H](F)Cl)C1]

R⁷ is methyl;
R² is isopropyl;
R³ is ethyl;
R⁴ is

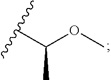 [SMILES: *[C@H](C)OC]

R⁵ is H or methylpiperazinyl;
A¹ is

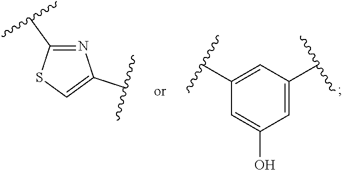 [SMILES: *c1csc(*)n1 ; *c1cc(*)cc(O)c1]

A² is dimethylmethylene;
A³ is O;
or a pharmaceutically acceptable salt thereof.

The present invention relates to (i′″) a compound of formula (I),

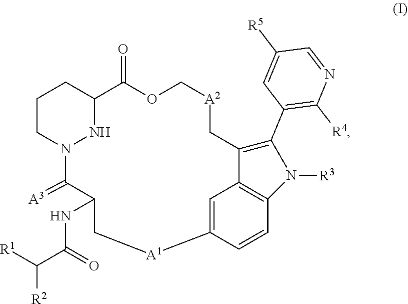

wherein
R¹ is

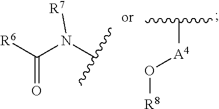

wherein R⁶ is pyrrolidinyl substituted by (C₁₋₆alkyl)₂oxooxetanylcarbonyl, (C₁₋₆alkylcarbonyl)carbonyl, (dihaloC₁₋₆alkyl)carbonyl, (oxooxetanylamino)carbonyl, C₂₋₆alkynyl, C₃₋₈alkadienylcarbonyl, cyanoC₁₋₆alkyl, cycloalkylcarbonyl, oxoazetidinylcarbonyl, pyridinylC₂₋₆alkynylcarbonyl or triazolylC₂₋₆alkenylcarbonyl; or cycloalkyl substituted by formyl, C₂₋₆alkynyl, C₁₋₆alkylsulfonylC₂₋₆alkenyl, (C₁₋₆alkyl)₂phosphorylC₂₋₆alkenyl or [(C₁₋₆alkyl)₂(oxo)-λ⁶-sulfanylidene]C₁₋₆alkylcarbonyl;
   R⁷ is C₁₋₆alkyl;
   R⁸ is ((dihaloC₁₋₆alkyl)carbonyl)azetidinyl;
   A⁴ is C₁₋₆alkylene;
R² is C₁₋₆alkyl;
R³ is C₁₋₆alkyl;
R⁴ is C₁₋₆alkoxyC₁₋₆alkyl;
R⁵ is H, (C₁₋₆alkyl)₂phosphoryl, C₁₋₆alkylpiperazinyl or C₁₋₆alkylsulfonimidoyl;
A¹ is thiazolylene or phenylene substituted by R⁹; wherein R⁹ is H, hydroxy, (C₁₋₆alkyl)₂phosphoryl or C₁₋₆alkylsulfonimidoyl;
A² is C₁₋₆alkylene;
A³ is O or S;
or a pharmaceutically acceptable salt thereof.

A further embodiment of present invention is (ii′″) a compound of formula (I) according to (i′″), wherein
R¹ is

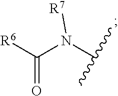

wherein R⁶ is pyrrolidinyl substituted by (C₁₋₆alkylcarbonyl)carbonyl, (dihaloC₁₋₆alkyl)carbonyl, C₂₋₆alkynyl or cyanoC₁₋₆alkyl; or cycloalkyl substituted by formyl, C₂₋₆alkynyl or [(C₁₋₆alkyl)₂(oxo)-λ⁶-sulfanylidene]C₁₋₆alkylcarbonyl;
R⁷ is C₁₋₆alkyl;
R² is C₁₋₆alkyl;
R³ is C₁₋₆alkyl;
R⁴ is C₁₋₆alkoxyC₁₋₆alkyl;
R⁵ is H;
A¹ is phenylene substituted by R⁹; wherein R⁹ is hydroxy;
A² is C₁₋₆alkylene;
A³ is O;
or a pharmaceutically acceptable salt thereof.

A further embodiment of present invention is (iii′″) a compound of formula (I) according to (i′″) or (ii′″), or a pharmaceutically acceptable salt thereof, wherein
R¹ is

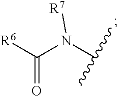

wherein R⁶ is pyrrolidinyl substituted by (dihaloC₁₋₆alkyl)carbonyl, or cycloalkyl substituted by formyl, C₂₋₆alkynyl or [(C₁₋₆alkyl)₂(oxo)-λ⁶-sulfanylidene]C₁₋₆alkylcarbonyl;
R⁷ is C₁₋₆alkyl.

A further embodiment of present invention is (iv′″) a compound of formula (I), according to any one of (i′″) to (iii′″), or a pharmaceutically acceptable salt thereof, wherein
R¹ is

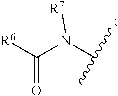

wherein R⁶ is pyrrolidinyl substituted by chloro(fluoro)acetyl, or cycloalkyl substituted by formyl, ethynyl or [dimethyl(oxo)-λ⁶-sulfanylidene]acetyl;
R⁷ is methyl.

A further embodiment of present invention is (v′″) a compound of formula (I) according to any one of (i′″) to (iv′″), wherein
R¹ is

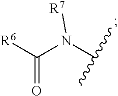

wherein R⁶ is

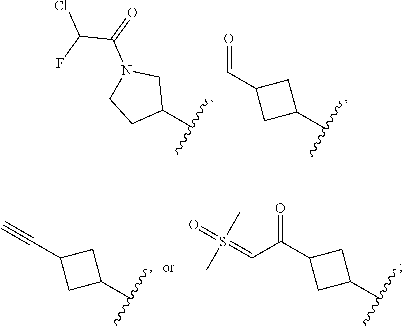

R⁷ is methyl.

A further embodiment of present invention is (vi′″) a compound of formula (I), or a pharmaceutically acceptable salt thereof, according to any one of (i′″) to (v′″), wherein R² is isopropyl.

A further embodiment of present invention is (vii′″) a compound of formula (I), or a pharmaceutically acceptable salt thereof, according to any one of (i″) to (vi′″), wherein R³ is ethyl.

A further embodiment of present invention is (viii′″) a compound of formula (I), or a pharmaceutically acceptable salt thereof, according to any one of (i′″) to (vii′″), wherein R⁴ is methoxyethyl.

A further embodiment of present invention is (ix′″) a compound of formula (I), or a pharmaceutically acceptable salt thereof, according to any one of (i′″) to (viii′″), wherein R⁵ is H.

A further embodiment of present invention is (x′″) a compound of formula (I), or a pharmaceutically acceptable salt thereof, according to any one of (i′″) to (ix′″), wherein A¹ is

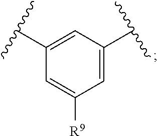

wherein R⁹ is hydroxy.

A further embodiment of present invention is (xi′″) a compound of formula (I), or a pharmaceutically acceptable salt thereof, according to any one of (i′″) to (x′″), wherein A² is

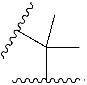 [SMILES: *C(*)(C)C]

A further embodiment of present invention is (xii′″) a compound of formula (I), according to any one of (i′″) to (xi′″), wherein
R¹ is

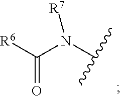

wherein R⁶ is pyrrolidinyl substituted by (dihaloC₁₋₆alkyl)carbonyl, or cycloalkyl substituted by formyl, C₂₋₆alkynyl or [(C₁₋₆alkyl)₂(oxo)-λ⁶-sulfanylidene]C₁₋₆alkylcarbonyl;
R⁷ is C₁₋₆alkyl;
R² is C₁₋₆alkyl;
R³ is C₁₋₆alkyl;
R⁴ is C₁₋₆alkoxyC₁₋₆alkyl; R⁵ is H;
A¹ is phenylene substituted by R⁹; wherein R⁹ is hydroxy;
A² is C₁₋₆alkylene;
A³ is O;
or a pharmaceutically acceptable salt thereof.

A further embodiment of present invention is (xii′″) a compound of formula (I), according to any one of (i′″) to (xi′″), wherein
R¹ is

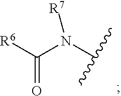

wherein R⁶ is

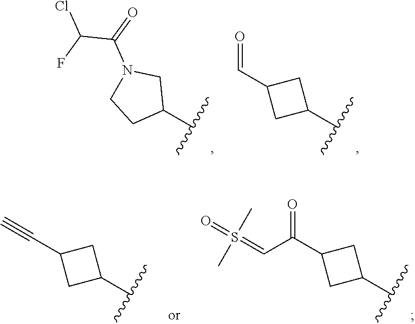

R⁷ is methyl;
R² is isopropyl;
R³ is ethyl;
R⁴ is methoxyethyl;
R⁵ is H;
A¹ is

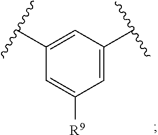

wherein R⁹ is hydroxy;
A² is

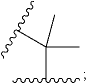 [SMILES: *C(*)(C)C]

A³ is O;
or a pharmaceutically acceptable salt thereof.

Another embodiment of present invention is a compound of formula (I) selected from the following:
(3S)-1-[(2S)-2-chloro-2-fluoro-acetyl]-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-pyrrolidine-3-carboxamide;
(3S)-1-[(2R)-2-chloro-2-fluoro-acetyl]-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-pyrrolidine-3-carboxamide;
N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-3-formyl-N-methyl-cyclobutanecarboxamide;
N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-3-ethynyl-N-methyl-cyclobutanecarboxamide;
(3S)-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-1-(2-oxopropanoyl)pyrrolidine-3-carboxamide;
(3S)-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-1-prop-2-ynyl-pyrrolidine-3-carboxamide;
(3S)-1-(cyanomethyl)-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-pyrrolidine-3-carboxamide;
3-[2-[dimethyl(oxo)-λ⁶-sulfanylidene]acetyl]-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-cyclobutanecarboxamide;
(3S)-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-1-[(E)-3-(1,2,4-triazol-1-yl)prop-2-enoyl]pyrrolidine-3-carboxamide;
(3S)-1-[(2R)-2-chloro-2-fluoro-acetyl]-N-[(1S)-1-[[(7S,13S)-21-ethyl-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.12,5.19,13.022,26]octacosa-1(25),2,5(28),19,22(26),23-hexaen-7-yl]carbamoyl]-2-methyl-propyl]-N-methyl-pyrrolidine-3-carboxamide;
(3S)-1-(bicyclo[1.1.0]butane-1-carbonyl)-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-pyrrolidine-3-carboxamide;
(3S)-1-[(2R)-2-chloro-2-fluoro-acetyl]-N-[(1S)-1-[[(8S,14S)-22-ethyl-4-hydroxy-(21M)-21-[2-[(1S)-1-methoxyethyl]-5-(4-methylpiperazin-1-yl)-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-pyrrolidine-3-carboxamide;
N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-3-[2-(2-pyridyl)ethynyl]cyclobutanecarboxamide;
N-[(1S)-1[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-3-ethynyl-N-methyl-azetidine-1-carboxamide;
N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-4-ethynyl-N-methyl-piperidine-1-carboxamide;
N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-4-ethynyl-4-fluoro-N-methyl-piperidine-1-carboxamide;
(3S)-1-[(2R)-2-chloro-2-fluoro-acetyl]-N-[(1S)-1-[[(7S,13S)-21-ethyl-(20M)-20-[2-[(1S)-1-methoxyethyl]-3-pyridyl]-17,17-dimethyl-8,14-dioxo-15-oxa-4-thia-9,21,27,28-tetrazapentacyclo[17.5.2.12,5.19,13.022,26]octacosa-1(25),2,5(28),19,22(26),23-hexaen-7-yl]carbamoyl]-2-methyl-propyl]-N-methyl-pyrrolidine-3-carboxamide;
(2S)-2-[2-[(2R)-2-chloro-2-fluoro-acetyl]-6-oxo-2,7-diazaspiro[4.5]decan-7-yl]-N-[(7S,13S)-21-ethyl-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.12,5.19,13.022,26]octacosa-1(25),2,5(28),19,22(26),23-hexaen-7-yl]-3-methyl-butanamide;
(2S)-2-[2-[(2R)-2-chloro-2-fluoro-acetyl]-6-oxo-2,7-diazaspiro[4.5]decan-7-yl]-N-[(7S,13S)-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.12,5.19,13.022,26]octacosa-1(25),2,5(28),19,22(26),23-hexaen-7-yl]-3-methyl-butanamide;
1-[(2R)-2-chloro-2-fluoro-acetyl]-4-fluoro-N-[(1S)-1-[[(7S,13S)-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.12,5.19,13.022,26]octacosa-1(25),2,5(28),19,22(26),23-hexaen-7-yl]carbamoyl]-2-methyl-propyl]-N-methyl-piperidine-4-carboxamide;
(3S)-1-[(2R)-2-chloro-2-fluoro-acetyl]-N-[(1S)-1-[[(7S,13S)-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.12,5.19,13.022,26]octacosa-1(25),2,5(28),19,22(26),23-hexaen-7-yl]carbamoyl]-2-methyl-propyl]-N-methyl-pyrrolidine-3-carboxamide;
(3S)-1-[(2R)-2-chloro-2-fluoro-acetyl]-N-[(1S)-1-[[(7S,13S)-(20M)-20-[2-[(1S)-1-methoxyethyl]-3-pyridyl]-17,17-dimethyl-8,14-dioxo-21-(2,2,2-trifluoroethyl)-15-oxa-4-thia-9,21,27,28-tetrazapentacyclo[17.5.2.12,5.19,13.022,26]octacosa-1(25),2,5(28),19,22(26),23-hexaen-7-yl]carbamoyl]-2-methyl-propyl]-N-methyl-pyrrolidine-3-carboxamide;
(2S)-2-[(5S)-7-[(2R)-2-chloro-2-fluoro-acetyl]-1-oxo-2,7-diazaspiro[4.4]nonan-2-yl]-N-[(7S,13S)-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.12,5.19,13.022,26]octacosa-1(25),2,5(28),19,22(26),23-hexaen-7-yl]-3-methyl-butanamide;
(2S)-2-[(5R)-7-[(2R)-2-chloro-2-fluoro-acetyl]-1-oxo-2,7-diazaspiro[4.4]nonan-2-yl]-N-[(7S,13S)-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.12,5.19,13.022,26]octacosa-1(25),2,5(28),19,22(26),23-hexaen-7-yl]-3-methyl-butanamide;
(3S)-1-[(2R)-2-chloro-2-fluoro-acetyl]-N-[(1S)-1-[[(7S,13S)-(20M)-20-[2-[(1S)-1-methoxyethyl]-5-morpholino-3-pyridyl]-17,17-dimethyl-8,14-dioxo-21-(2,2,2-trifluoroethyl)-15-oxa-4-thia-9,21,27,28-tetrazapentacyclo[17.5.2.12,5.19,13.022,26]octacosa-1(25),2,5(28),19,22(26),23-hexaen-7-yl]carbamoyl]-2-methyl-propyl]-N-methyl-pyrrolidine-3-carboxamide; and
(3S)-1-[(2R)-2-chloro-2-fluoro-acetyl]-N-[(1S)-1-[[(7S,13S)-21-ethyl-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.12,5.19,13.022,26]octacosa-1(25),2,5(28),19,22(26),23-hexaen-7-yl]carbamoyl]-2-methyl-propyl]-N-methyl-pyrrolidine-3-carboxamide;
   or a pharmaceutically acceptable salt thereof.

Another embodiment of present invention is (xvi) compound RM461-A with structure of

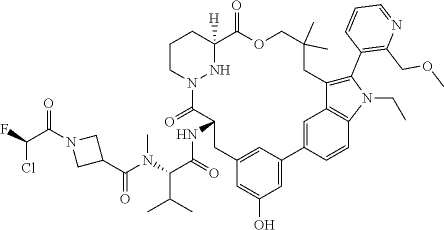 [SMILES: CCn1c(-c2cccnc2COC)c2c3cc(ccc31)-c1cc(O)cc(c1)C[C@H](NC(=O)[C@H](C(C)C)N(C)C(=O)C1CN(C(=O)[C@H](F)Cl)C1)C(=O)N1CCC[C@H](N1)C(=O)OCC(C)(C)C2]

or a pharmaceutically acceptable salt thereof.

Another embodiment of present invention is related to (xvii) a process for the preparation of a compound according to any one of (i) to (xv), (i′) to (xiv′), (i″) to (xiii″) or (i′″) to (xii′″) comprising any of the following steps:
a) coupling reaction between compound of formula (IX),

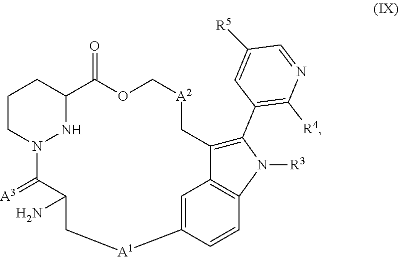

and acid (X),

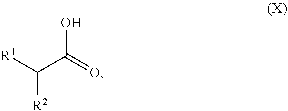

with coupling reagent in the presence of a base;
b) coupling reaction between compound of formula (XIII),

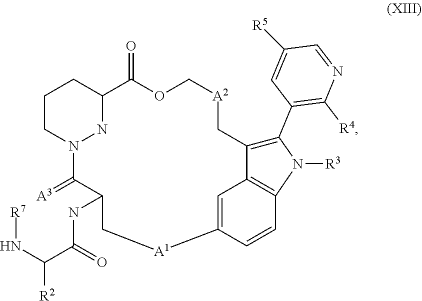

and acid (XIV),

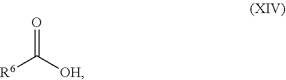

with coupling reagent in the presence of a base;
c) coupling reaction between compound of formula (XVIII),

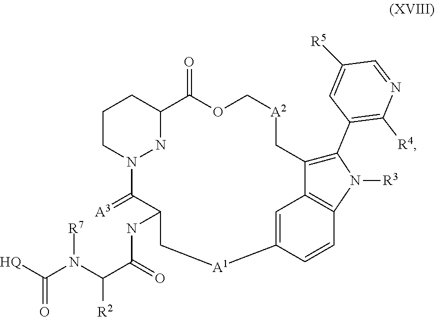

and acid (XIX),

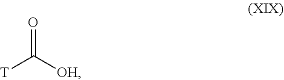

with coupling reagent in the presence of a base;
wherein Q is heterocyclylene; T is (C₁₋₆alkyl)₂oxooxetanyl, C₁₋₆alkylcarbonyl, dihaloC₁₋₆alkyl, oxooxetanylamino, C₂₋₆alkynyl, C₃₋₈alkadienyl, cyanoC₁₋₆alkyl, cycloalkyl, morpholinylC₂₋₆alkynyl, oxoazetidinyl, pyridinylC₂₋₆alkynyl or triazolylC₂₋₆alkenyl; R¹, R², R³, R⁴, R⁵, R⁶, R⁷, A¹, A² and A³ are defined as in any one of claims 1 to 14; the coupling reagent is T₃P, HATU, PyBOP or EDCI/HOBt; the base is TEA, DIEPA or DMAP.

Another embodiment of present invention is (xviii) a compound or pharmaceutically acceptable salt according to any one of (i) to (xvi) for use as therapeutically active substance.

Another embodiment of present invention is (xix) a pharmaceutical composition comprising a compound in according to any one of (i) to (xvi) and a therapeutically inert carrier.

Another embodiment of present invention is (xx) the use of a compound according to any one of (i) to (xvi) for treating a KRAS G12C protein-related disease.

Another embodiment of present invention is (xxi) the use of a compound according to any one of (i) to (xvi) for treating a KRAS G12C, G12D and G12V protein-related disease.

Another embodiment of present invention is (xxii) the use of a compound according to any one of (i) to (xvi) for inhibiting RAS interaction with downstream effectors, wherein the downstream effectors are RAF and PI3K.

Another embodiment of present invention is (xxiii) the use of a compound according to any one of (i) to (xvi) for inhibiting the propagating oncogenic MAPK and PI3K signaling.

Another embodiment of present invention is (xxiv) the use of a compound according to any one of (i) to (xvi) for the treatment or prophylaxis of KRAS mutation driven cancers, wherein the cancer is selected from pancreatic cancer, colorectal cancer, lung cancer, esophageal cancer, gallbladder cancer, melanoma ovarian cancer and endometrial cancer.

Another embodiment of present invention is (xxv) the use of a compound according to any one of (i) to (xvi) for the treatment or prophylaxis of KRAS mutation driven cancers, wherein the cancer is selected from pancreatic adenocarcinoma, colorectal cancer and non-small cell lung cancer.

Another embodiment of present invention is (xxvi) a compound or pharmaceutically acceptable salt according to any one of (i) to (xvi) for the treatment or prophylaxis of KRAS mutation driven cancers, wherein the cancer is selected from pancreatic adenocarcinoma, colorectal cancer and non-small cell lung cancer.

Another embodiment of present invention is (xxvii) the use of a compound according to any one of (i) to (xvi) for the preparation of a medicament for the treatment or prophylaxis of KRAS mutation driven cancers, wherein the cancer is selected from pancreatic adenocarcinoma, colorectal cancer and non-small cell lung cancer.

Another embodiment of present invention is (xxviii) a method for the treatment or prophylaxis of KRAS mutation driven cancers, wherein the cancer is selected from pancreatic adenocarcinoma, colorectal cancer and non-small cell lung cancer, which method comprises administering a therapeutically effective amount of a compound as defined in any one of (i) to (xvi).

Another embodiment of present invention is (xxix) a compound or pharmaceutically acceptable salt according to any one of (i) to (xvi), when manufactured according to a process of (xvii).

### Pharmaceutical Compositions and Administration

Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compounds of formula (I) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to inhibit mutant RAS (e.g. KRAS G12C) interaction with RAF, blocking the oncogenic MAPK signaling. For example, such amount may be below the amount that is toxic to normal cells, or the mammal as a whole.

In one example, the pharmaceutically effective amount of the compound of the invention administered parenterally per dose will be in the range of about 0.1 to 1000 mg/kg, alternatively about 0.1 to 1000 mg/kg of patient body weight per day, with the typical initial range of compound used being 0.3 to 15 mg/kg/day. In another embodiment, oral unit dosage forms, such as tablets and capsules, preferably contain from about 1 to about 1000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 1 to 1000 mg of the compound of the invention compounded with about 1 to 1000 mg anhydrous lactose, about 1 to 1000 mg sodium croscarmellose, about 1 to 1000 mg polyvinylpyrrolidone (PVP) K30, and about 1 to 1000 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5 to 400 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of formula (I), or a stereoisomer or pharmaceutically acceptable salt thereof. In a further embodiment includes a pharmaceutical composition comprising a compound of formula (I), or a stereoisomer or pharmaceutically acceptable salt thereof, together with a pharmaceutically acceptable carrier or excipient.

Another embodiment includes a pharmaceutical composition comprising a compound of formula (I) for use in the treatment of mutant KRAS-driven cancers. Another embodiment includes a pharmaceutical composition comprising a compound of Formula (I) for use in the treatment of mutant KRAS-driven cancers.

The following example A and B illustrate typical compositions of the present invention, but serve merely as representative thereof.

### Example A

A compound of the present invention can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Example B

A compound of the present invention can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

### Indications and Methods of Treatment

The compounds of the invention induce a new binding pocket in KRAS by driving formation of a high affinity tri-complex between KRAS protein and the widely expressed cyclophilin A (CYPA), which inhibit KRAS interaction with downstream effectors, such as RAF and PI3K. Accordingly, the compounds of the invention are useful for inhibiting the propagating oncogenic MAPK and PI3K signaling, reducing cell proliferation, in particular cancer cells. Compounds of the invention are useful for termination of RAS signaling in cells that express RAS mutant, e.g. KRAS mutation driven pancreatic cancer, colorectal cancer, lung cancer, esophageal cancer, gallbladder cancer, melanoma ovarian cancer, endometrial cancer, etc. Alternatively, compounds of the invention are useful for termination of RAS signaling in malignant solid tumor where the oncogenic role of KRAS mutation is reinforced by dysregulation or mutation of effector pathways as MAPK, PI3K-AKT-mTOR (Mammalian target of rapamycin) driven signaling, for targeted therapy in pancreatic adenocarcinoma, colorectal cancer, non-small cell lung cancer, etc.

Another embodiment includes a method of treating or preventing cancer in a mammal in need of such treatment, wherein the method comprises administering to said mammal a therapeutically effective amount of a compound of formula (I), a stereoisomer, tautomer, prodrug or pharmaceutically acceptable salt thereof.

### Synthesis

The compounds of the present invention can be prepared by any conventional means. Suitable processes for synthesizing these compounds as well as their starting materials are provided in the schemes below and in the examples. All substituents, in particular, R¹ to R⁵ and A¹ to A³ are as defined above unless otherwise indicated. Furthermore, and unless explicitly otherwise stated, all reactions, reaction conditions, abbreviations and symbols have the meanings well known to a person of ordinary skill in organic chemistry.

General synthetic routes for preparing the compound of formula (I) are shown below.

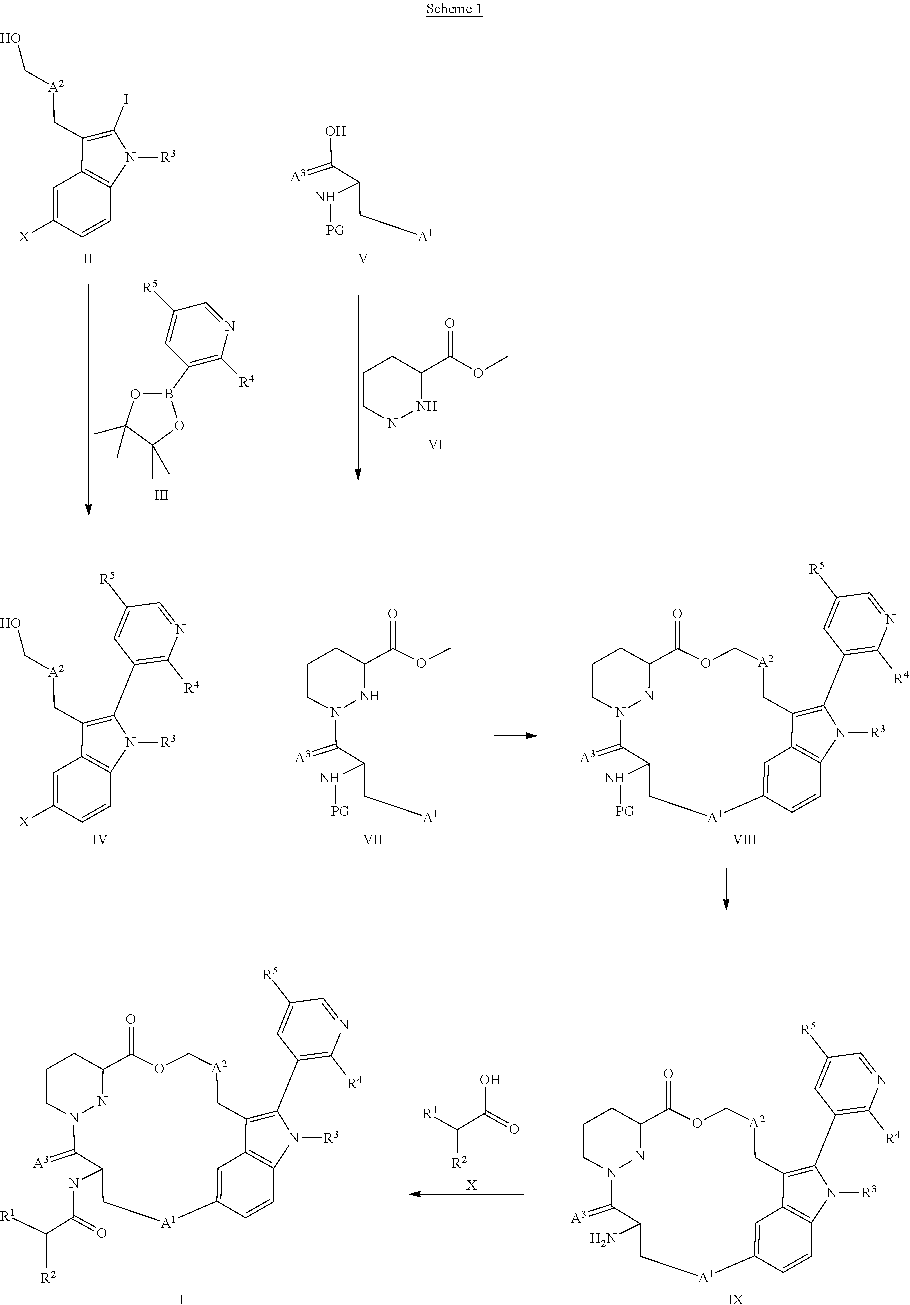

wherein X is halogen; PG is a protecting group, such as Boc and Cbz; Q is heterocyclylene.

The coupling of compound of formula (II) with compound (III) can be achieved under standard Suzuki coupling conditions to provide compound of formula (IV). Compound of formula (VII) can be obtained by a coupling reaction between acid (V) and compound (VI) with coupling reagent(s), such as T₃P, HATU, PyBOP and EDCI/HOBt, in the presence of a base, such as TEA, DIEPA and DMAP. The coupling of compound of formula (IV) with compound of formula (VII) can be achieved under Suzuki coupling conditions to provide compound of formula (VIII). Deprotection of compound of formula (VIII) can afford compound of formula (IX) in the presence of an acid, such as TFA, or under hydrogenation condition with a catalyst, such as Pd/C and Pd(OH)₂/C. Compound of formula (I) can be obtained by a coupling reaction between acid (X) and compound of formula (IX) with coupling reagent(s), such as T₃P, HATU, PyBOP and EDCI/HOBt, in the presence of a base, such as TEA, DIEPA and DMAP.

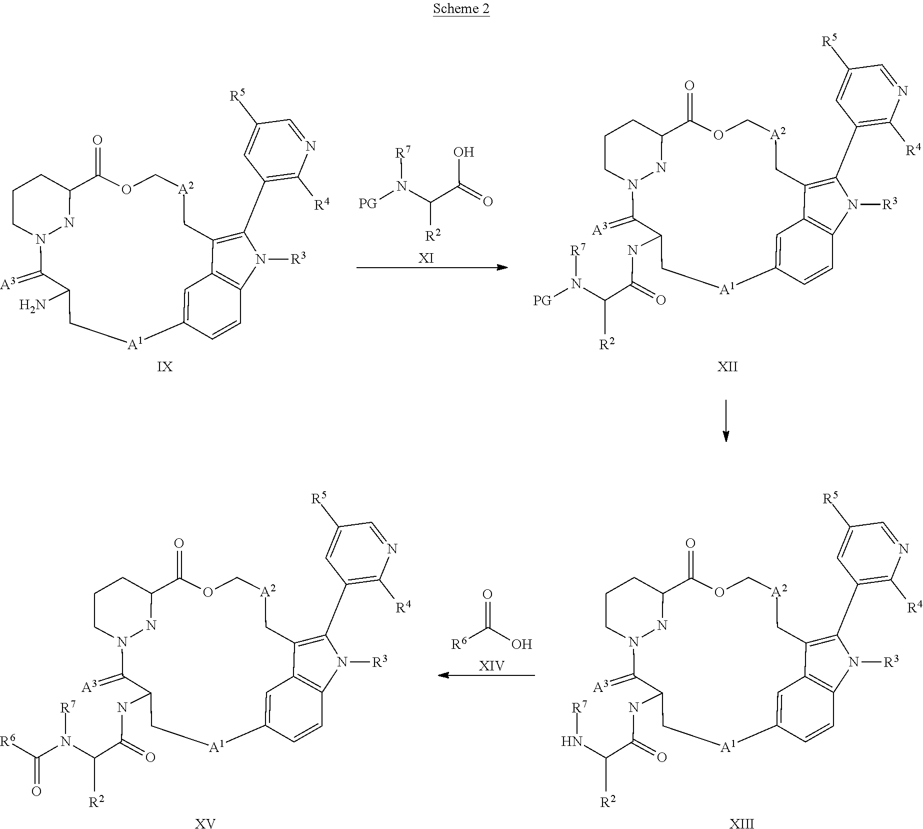

wherein X is halogen; PG is a protecting group, such as Boc and Cbz; Q is heterocyclylene.

Alternatively compound of formula (XII) can be obtained by a coupling reaction between acid (XI) and compound of formula (IX) with coupling reagent(s), such as T₃P, HATU, PyBOP or EDCI/HOBt, in the presence of a base, such TEA, DIEPA or DMAP. Deprotection of compound of formula (XII) can afford compound of formula (XIII) in the presence of an acid, such as TFA, or under hydrogenation condition with a catalyst, such as Pd/C and Pd(OH)₂/C. Compound of formula (XV) can be obtained by a coupling reaction between acid (XIV) and compound of formula (XIII) with coupling reagent(s), such as T₃P, HATU, PyBOP and EDCI/HOBt, in the presence of a base, such as TEA, DIEPA and DMAP.

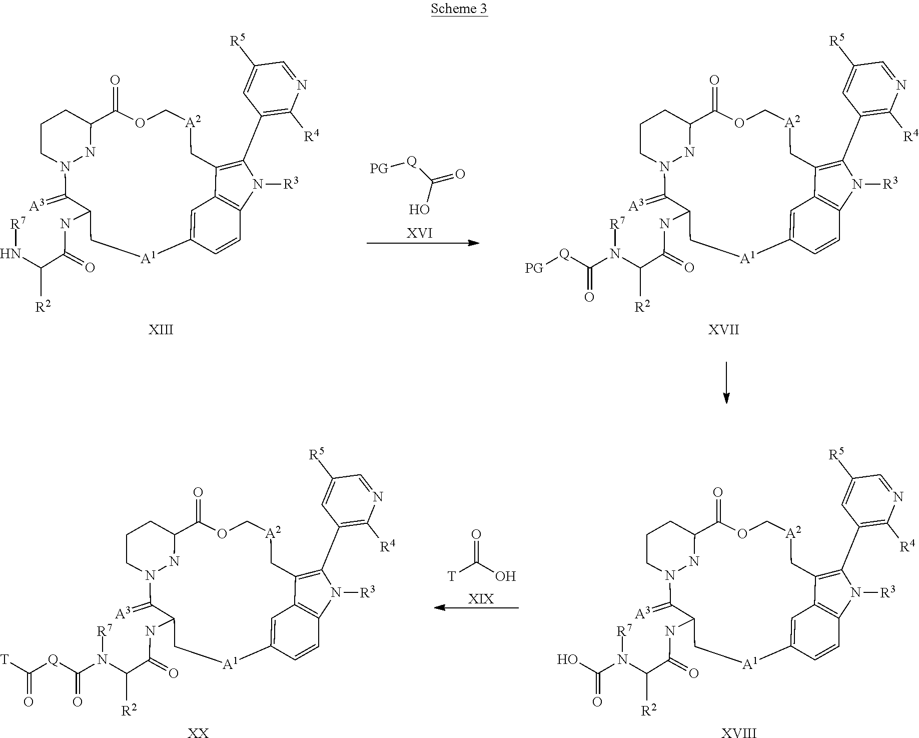

wherein X is halogen; PG is a protecting group, such as Boc and Cbz; Q is heterocyclylene; T is (C₁₋₆alkyl)₂oxooxetanyl, C₁₋₆alkylcarbonyl, dihaloC₁₋₆alkyl, oxooxetanylamino, C₂₋₆alkynyl, C₃₋₈alkadienyl, cyanoC₁₋₆alkyl, cycloalkyl, oxoazetidinyl, pyridinylC₂₋₆alkynyl or triazolylC₂₋₆alkenyl.

Alternatively compound of formula (XVII) can be obtained by a coupling reaction using acid (XVI), compound of formula (XIII) and coupling reagent(s), such as T₃P, HATU, PyBOP or EDCI/HOBt, in the presence of a base, such TEA, DIEPA or DMAP. Deprotection of compound of formula (XVII) can afford compound of formula (XVIII) in the presence of an acid, such as TFA, or under hydrogenation condition with a catalyst, such as Pd/C and Pd(OH)₂/C. Compound of formula (XX) can be obtained by a coupling reaction between acid (XIX) and compound of formula (XVIII) with coupling reagent(s), such as T₃P, HATU, PyBOP and EDCI/HOBt, in the presence of a base, such as TEA, DIEPA and DMAP.

Compounds of this invention can be obtained as mixtures of diastereomers or enantiomers, which can be separated by methods well known in the art, e.g. (chiral) HPLC or SFC. In another embodiment, compound of formula (I) can be obtained according to above scheme by using corresponding chiral starting materials.

This invention also relates to a process for the preparation of a compound of formula (I) comprising any of the following steps:
a) coupling reaction between compound of formula (IX),

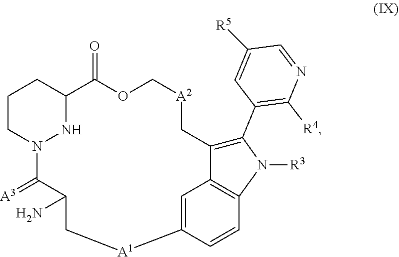

and acid (X),

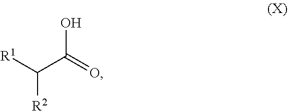

with coupling reagent in the presence of a base;
b) coupling reaction between compound of formula (XIII),

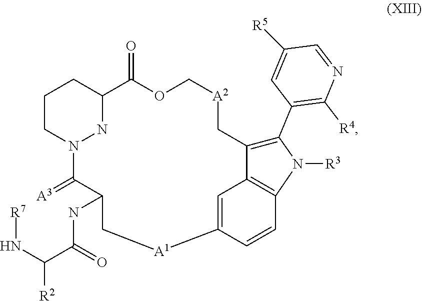

and acid (XIV),

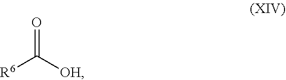

with coupling reagent in the presence of a base;
c) coupling reaction between compound of formula (XVIII),

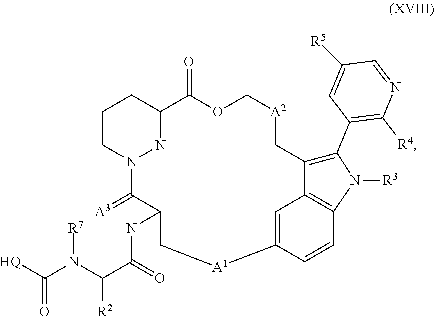

and acid (XIX),

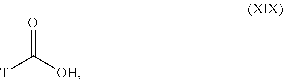

with coupling reagent in the presence of a base;
wherein
in step a), b) and c) the coupling reagent can be, for example, T₃P, HATU, PyBOP or EDCI/HOBt; the base can be, for example, TEA, DIEPA or DMAP.

A compound of formula (I) when manufactured according to the above process is also an object of the invention.

### EXAMPLES

The invention will be more fully understood by reference to the following examples. They should not, however, be construed as limiting the scope of the invention.

### Abbreviations

The invention will be more fully understood by reference to the following examples. They should not, however, be construed as limiting the scope of the invention.

Abbreviations used herein are as follows:
ACN acetonitrile
aq. Aqueous
CDCl₃: deuterated chloroform
CD₃OD: deuterated methanol
DIEPA: N, N-diethylpropylamine
DMAP: 4-Dimethylaminopyridine
DMF: dimethyl formamide
DMSO: dimethyl sulfoxide
EDCI: N-Ethyl-N′-(3-dimethylaminopropyl)carbodiimide hydrochloride
EtOAc or EA: ethyl acetate
FRET fluorescence resonance energy transfer
HATU: (1-[Bis(dimethylamino)methylene]-1H-1,2,3-triazolo[4,5-b]pyridinium 3-oxid hexafluorophosphate)
hr(s): hour(s)
HPLC: high performance liquid chromatography
HOBt: N-hydroxybenzotriazole
MS: (ESI): mass spectroscopy (electron spray ionization)
min(s) minute(s)
NMR: nuclear magnetic resonance
obsd. observed
prep-HPLC preparative high performance liquid chromatography
PyBOP: benzotriazol-1-yloxytripyrrolidinophosphonium hexafluorophosphate
RT or rt: room temperature
sat. saturated
SFC supercritical fluid chromatography
TEA: triethylamine
TFA: trifluoroacetic acid
THF: tetrahydrofuran
TEA: triethylamine
T₃P: propylphosphonic anhydride

### General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using XBridge™ Prep-C18 (5 μm, OBD™ 30×100 mm) column, SunFire™ Prep-C18 (5 μm, OBD™ 30×100 mm) column, Phenomenex Synergi-C18 (10 μm, 25×150 mm) or Phenomenex Gemini-C18 (10 μm, 25×150 mm). Waters AutoP purification System (Sample Manager 2767, Pump 2525, Detector: Micromass ZQ and UV 2487, solvent system: acetonitrile and 0.1% ammonium hydroxide in water; acetonitrile and 0.1% FA in water or acetonitrile and 0.1% TFA in water). Or Gilson-281 purification System (Pump 322, Detector: UV 156, solvent system: acetonitrile and 0.05% ammonium hydroxide in water; acetonitrile and 0.225% FA in water; acetonitrile and 0.05% HCl in water; acetonitrile and 0.075% TFA in water; or acetonitrile and water).

For SFC chiral separation, intermediates were separated by chiral column (Daicel chiralpak IC, 5 μm, 30×250 mm), AS (10 μm, 30×250 mm) or AD (10 μm, 30×250 mm) using Mettler Toledo Multigram III system SFC, Waters 80Q preparative SFC or Thar 80 preparative SFC, solvent system: CO₂ and IPA (0.5% TEA in IPA) or CO₂ and MeOH (0.1% NH₃·H₂O in MeOH), back pressure 100 bar, detection UV@254 or 220 nm.

LC/MS spectra of compounds were obtained using a LC/MS (Waters™ Alliance 2795-Micromass ZQ, Shimadzu Alliance 2020-Micromass ZQ or Agilent Alliance 6110-Micromass ZQ), LC/MS conditions were as follows (running time 3 or 1.5 mins):
Acidic condition I: A: 0.1% TFA in H₂O; B: 0.1% TFA in acetonitrile;
Acidic condition II: A: 0.0375% TFA in H₂O; B: 0.01875% TFA in acetonitrile;
Basic condition I: A: 0.1% NH₃—H₂O in H₂O; B: acetonitrile;
Basic condition II: A: 0.025% NH₃—H₂O in H₂O; B: acetonitrile;
Neutral condition: A: H₂O; B: acetonitrile.
Mass spectra (MS): generally only ions which indicate the parent mass are reported, and unless otherwise stated the mass ion quoted is the positive mass ion (MH)⁺.

NMR Spectra were obtained using Bruker Avance 400 MHz.

The microwave assisted reactions were carried out in a Biotage Initiator Sixty microwave synthesizer. All reactions involving air-sensitive reagents were performed under an argon or nitrogen atmosphere. Reagents were used as received from commercial suppliers without further purification unless otherwise noted.

### Preparative Examples

### Preparation of Intermediate

### Intermediate A

### 3-[5-bromo-1-ethyl-(2M)-2-[2-[(1S)-1-methoxyethyl]-3-pyridyl]indol-3-yl]-2,2-dimethyl-propan-1-ol

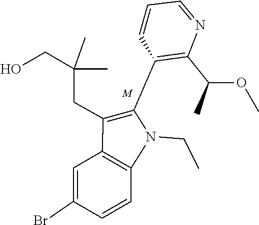

The title intermediate A was prepared according to the following scheme:

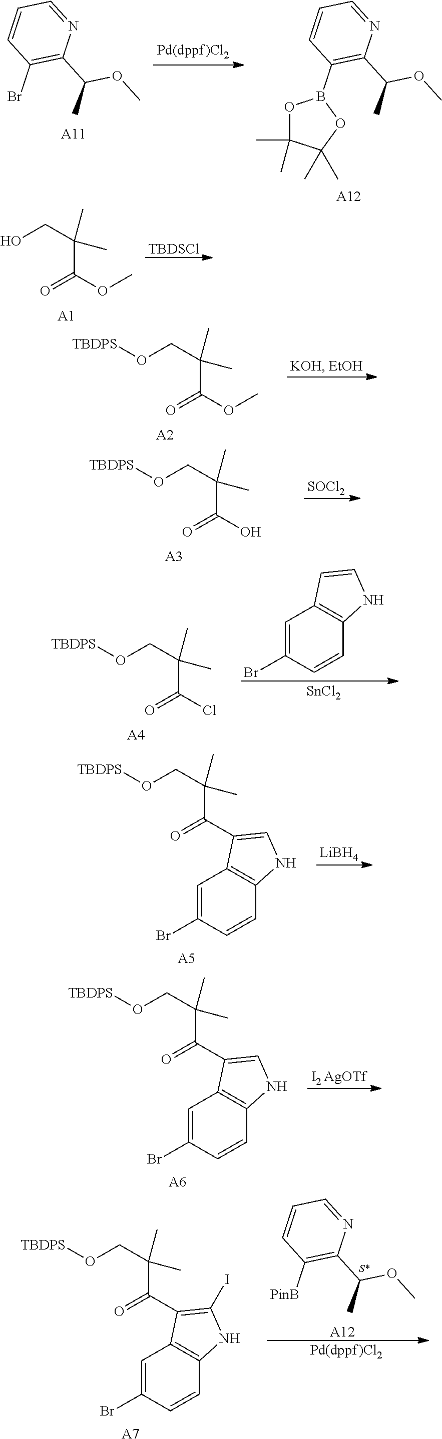

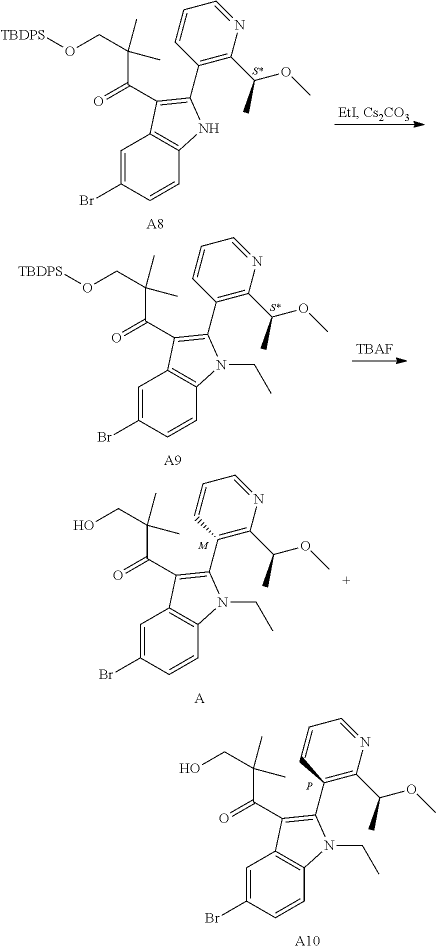

### Preparation of 2-[(1S)-1-methoxyethyl]-3-(4,4,5,5-tetramethyl-1,3,2-dioxaborolan-2-yl)pyridine (compound A12)

To a mixture of 3-bromo-2-[(1S)-1-methoxyethyl]pyridine (compound A11, 40.0 g, 185.1 mmol) and bis(pinacolato)diboron (56.4 g, 222.1 mmol) in 1,4-dioxane (500 mL) was added KOAc (23.1 mL, 370.2 mmol) and [1,1′-bis(diphenylphosphino)ferrocene]dichloropalladium(II) (6.7 g, 9.2 mmol). The mixture was stirred at 100° C. for 5 hrs under N₂ protection. TLC (Ethyl acetate) showed the starting material was consumed. The mixture was filtered and the filtrate was concentrated under vacuum. The residue was purified by silica gel chromatography (EA/PE: 0-50%) to afford 2-[(1S)-1-methoxyethyl]-3-(4,4,5,5-tetramethyl-1,3,2-dioxaborolan-2-yl)pyridine (compound A12, 55 g) as dark brown oil. MS: calc'd 264 (MH⁺), measured 264.1 (MH⁺).

### Step 1: Preparation of methyl 3-[tert-butyl(diphenyl)silyl]oxy-2,2-dimethyl-propanoate (compound A2)

To a solution of methyl 3-hydroxy-2,2-dimethylpropanoate (compound A1, 110.0 g, 832.3 mmol) and imidazole (169.9 g, 2497 mmol) in THF (1500 mL) was added tert-butylchlorodiphenylsilane (256.5 mL, 998.7 mmol) at 0° C. The mixture was stirred at 0° C. for 2 hrs. The mixture was diluted with petroleum ether (1000 mL) and filtered. The cake was washed with petroleum ether (150 mL) for 2 times. The combined filtrate was concentrated under vacuum to give a residue. The residue was purified by silica gel chromatography (EA/PE: 0-50%) to afford methyl 3-[tert-butyl(diphenyl)silyl]oxy-2,2-dimethyl-propanoate (compound A2, 220 g) as colorless oil. ¹H NMR (400 MHz, CDCl₃) δ ppm 7.68-7.63 (m, 4H), 7.44-7.36 (m, 6H), 3.69 (s, 3H), 3.65 (s, 2H), 1.21 (s, 6H), 1.04 (s, 9H).

### Step 2: Preparation of 3-[tert-butyl(diphenyl)silyl]oxy-2,2-dimethyl-propanoic acid (compound A3)

To a solution of methyl 3-[tert-butyl(diphenyl)silyl]oxy-2,2-dimethyl-propanoate (compound A2, 110.0 g, 296.8 mmol) in ethanol (1200 mL) was added a solution of potassium hydroxide (43.2 g, 770.2 mmol) in ethanol (500 mL). The mixture was stirred at 90° C. for 5 hrs. The mixture was concentrated under vacuum to remove EtOH and diluted with ice water (1000 mL). The mixture was acidified by 1 M aq. solution of HCl until pH=3. The aqueous phase was extracted with EtOAc (600 mL) for 2 times. The combined organic layer was washed with brine (400 mL), dried over anhydrous sodium sulfate, filtered. The filtrate was concentrated under vacuum. The residue was triturated with petroleum ether (300 mL) to afford 3-[tert-butyl(diphenyl)silyl]oxy-2,2-dimethyl-propanoic acid (compound A3, 80 g) as a white solid. ¹H NMR (400 MHz, CDCl₃) δ ppm 7.70-7.65 (m, 4H), 7.47-7.38 (m, 6H), 3.67 (s, 2H), 1.25 (s, 6H), 1.05 (s, 9H).

### Step 3: Preparation of 3-[tert-butyl(diphenyl)silyl]oxy-2,2-dimethyl-propanoyl chloride (compound A4)

To a solution of 3-[tert-butyl(diphenyl)silyl]oxy-2,2-dimethyl-propanoic acid (compound A3, 163.0 g, 457.1 mmol) and DMF (166.8 mg, 2.3 mmol) in DCM (50 mL) was added thionyl chloride (265.6 mL, 3657 mmol). The mixture was stirred at 50° C. for 12 hrs. The mixture was concentrated under vacuum to afford 3-[tert-butyl(diphenyl)silyl]oxy-2,2-dimethyl-propanoyl chloride (compound A4, 171.4 g) as yellow oil and it was used in the next step without further purification.

### Step 4: Preparation of 1-(5-bromo-1H-indol-3-yl)-3-[tert-butyl(diphenyl)silyl]oxy-2,2-dimethyl-propan-1-one (compound A5)

To a mixture of 3-[tert-butyl(diphenyl)silyl]oxy-2,2-dimethyl-propanoyl chloride (compound A4, 52.5 g, 140 mmol) in DCM (350 mL) was slowly added a solution of SnCl₄ (140 mL, 140 mmol) at 0° C. The mixture was stirred at −10° C. for 0.5 hour. Then 5-bromoindole (27.4 g, 140 mmol) in DCM (150 mL) was added dropwise. After addition, the mixture was stirred at −10° C. for 15 min. The mixture was diluted with sat. aq. solution NaHCO₃ (1000 mL) at 0° C., then extracted with EtOAc (800 mL) for 2 times. The combined organic layer was washed with brine (600 mL), dried over anhydrous sodium sulfate, filtered. The filtrate was concentrated under vacuum. The residue was triturated with EtOAc (100 mL) to afford 1-(5-bromo-1H-indol-3-yl)-3-[tert-butyl(diphenyl)silyl]oxy-2,2-dimethyl-propan-1-one (compound A5, 40 g) as a yellow solid. ¹H NMR (400 MHz, CDCl3) δ ppm 8.68 (s, 1H), 8.60 (br.s, 1H), 7.67 (d, J=2.8 Hz, 1H), 7.54-7.48 (m, 4H), 7.42-7.35 (m, 3H), 7.32-7.25 (m, 5H), 3.90 (s, 2H), 1.42 (s, 6H), 0.96 (s, 9H).

### Step 5: Preparation of [3-(5-bromo-1H-indol-3-yl)-2,2-dimethyl-propoxy]-tert-butyl-diphenyl-silane (compound A6)

To a solution of 1-(5-bromo-1H-indol-3-yl)-3-[tert-butyl(diphenyl)silyl]oxy-2,2-dimethyl-propan-1-one (compound A5, 20.0 g, 37.4 mmol) in THF (250 mL) was added LiBH₄ (28.1 mL, 112.2 mmol) at 0° C. under N₂ protection. The mixture was stirred at 60° C. for 12 hrs. Once the reaction was finished, the reaction was cooled to 25° C. and quenched with MeOH (20 mL). Then the mixture was diluted with EtOAc (300 mL) and washed with brine (250 mL). The organic layer was dried over anhydrous sodium sulfate, then filtered. The filtrate was concentrated under vacuum. The residue was dissolved in DCM (250 mL) and cooled to 10° C., to which diethyl 1,4-dihydro-2,6-dimethyl-3,5-pyridinedicarboxylate (3.7 g, 14.9 mmol) and p-toluenesulfonic acid monohydrate (356.7 mg, 1.8 mmol) were added. After being stirred at 10° C. for 2 hrs, the mixture was filtered and the filtrate was concentrated under vacuum. The residue was purified by silica gel chromatography (EA/PE: 0-50%) to afford [3-(5-bromo-1H-indol-3-yl)-2,2-dimethyl-propoxy]-tert-butyl-diphenyl-silane (compound A6, 17.2 g,) as colorless oil. ¹H NMR (400 MHz, CDCl3) δ 8.00 (s, 1H), 7.75 (s, 1H), 7.74-7.65 (m, 4H), 7.47-7.36 (m, 6H), 7.26-7.19 (m, 2H), 6.89 (d, J=2.0 Hz, 1H), 3.40 (s, 2H), 2.73 (s, 2H), 1.15 (s, 9H), 0.89 (s, 6H).

### Step 6: Preparation of [3-(5-bromo-2-iodo-1H-indol-3-yl)-2,2-dimethyl-propoxy]-tert-butyl-diphenyl-silane (compound A7)

To a solution of [3-(5-bromo-1H-indol-3-yl)-2,2-dimethyl-propoxy]-tert-butyl-diphenyl-silane (compound A6, 41.2 g, 79.1 mmol) and iodine (20.1 g, 79.1 mmol) in THF (500 mL) was added silver trifluoromethanesulfonate (24.4 g, 94.9 mmol). After being stirred at 25° C. for 1 hr, the mixture was quenched with sat. aq. solution of Na₂SO₃ (400 mL) and extracted with EtOAc (500 mL). The organic layer was washed with brine (400 mL), dried over anhydrous sodium sulfate, filtered and the filtrate was concentrated under vacuum. The residue was purified by silica gel chromatography (EA/PE: 0-20%) to afford [3-(5-bromo-2-iodo-1H-indol-3-yl)-2,2-dimethyl-propoxy]-tert-butyl-diphenyl-silane (compound A7, 46 g) as a yellow solid. ¹H NMR (400 MHz, CDCl3) δ ppm 8.07 (s, 1H), 7.75-7.67 (m, 5H), 7.46-7.37 (m, 6H), 7.26-7.15 (m, 2H), 3.49 (s, 2H), 2.70 (s, 2H), 1.15 (s, 9H), 0.94 (s, 6H). MS: calc'd 646 (MH⁺), measured 645.9 (MH⁺).

### Step 7: Preparation of [3-[5-bromo-2-[2-[(1S)-1-methoxyethyl]-3-pyridyl]-1H-indol-3-yl]-2,2-dimethyl-propoxy]-tert-butyl-diphenyl-silane (compound A8)

To a solution of [3-(5-bromo-2-iodo-1H-indol-3-yl)-2,2-dimethyl-propoxy]-tert-butyl-diphenyl-silane (compound A7, 18 g, 27.8 mmol) and 2-[(1S)-1-methoxyethyl]-3-(4,4,5,5-tetramethyl-1,3,2-dioxaborolan-2-yl)pyridine (compound A12, 10.9 g, 41.7 mmol) in 1,4-dioxane (200 mL) and water (30 mL) were added potassium carbonate (9.6 g, 69.6 mmol) and [1,1′-bis(diphenylphosphino)ferrocene]dichloropalladium(II) (1018.6 mg, 1.4 mmol). The mixture was stirred at 80° C. for 12 hrs under N₂. The mixture was cooled to 20° C., diluted with EtOAc (200 mL) and filtered. The filtrate was concentrated in vacuo. The residue was purified by silica gel chromatography (EA/PE: 0-20%) to afford [3-[5-bromo-2-[2-[(1S)-1-methoxyethyl]-3-pyridyl]-1H-indol-3-yl]-2,2-dimethyl-propoxy]-tert-butyl-diphenyl-silane (compound A8, 10 g) as brown oil. ¹H NMR (400 MHz, CDCl3) δ ppm 9.31 (s, 1H), 8.32 (dd, J=4.4, 0.8 Hz, 1H), 7.83-7.76 (m, 2H), 7.64-7.58 (m, 4H), 7.46-7.34 (m, 7H), 7.32-7.29 (m, 1H), 7.26-7.23 (m, 1H), 4.52 (q, J=6.4 Hz, 1H), 3.34 (s, 3H), 3.33-3.27 (m, 2H), 2.90-2.80 (m, 2H), 1.41 (d, J=6.4 Hz, 3H), 1.07 (s, 9H), 0.64 (s, 3H), 0.59 (s, 3H). MS: calc'd 657 (MH⁺), measured 657.0 (MH⁺).

### Step 8: Preparation of [3-[5-bromo-1-ethyl-2-[2-[(1S)-1-methoxyethyl]-3-pyridyl]indol-3-yl]-2,2-dimethyl-propoxy]-tert-butyl-diphenyl-silane (compound A9)

To a solution of [3-[5-bromo-2-[2-[(1S)-1-methoxyethyl]-3-pyridyl]-1H-indol-3-yl]-2,2-dimethyl-propoxy]-tert-butyl-diphenyl-silane (compound A8, 36.0 g, 54.9 mmol) in DMF (300 mL) were added cesium carbonate (35.7 g, 109.8 mmol) and iodoethane (8.7 mL, 109.8 mmol) at 0° C. After being stirred at 25° C. for 12 hrs, the mixture was diluted with water (1000 mL) and extracted with EtOAc (500 mL). The combined organic layer was washed with brine (200 mL), dried over anhydrous sodium sulfate, filtered and concentrated under vacuum to afford [3-[5-bromo-1-ethyl-2-[2-[(1S)-1-methoxyethyl]-3-pyridyl]indol-3-yl]-2,2-dimethyl-propoxy]-tert-butyl-diphenyl-silane (compound A9, 32 g) as brown oil. MS: calc'd 685 (MH⁺), measured 685.0 (MH⁺).

### Step 9: Preparation of 3-[5-bromo-1-ethyl-(2M)-2-[2-[(1S)-1-methoxyethyl]-3-pyridyl]indol-3-yl]-2,2-dimethyl-propan-1-ol (Intermediate A)

To a solution of [3-[5-bromo-1-ethyl-2-[2-[(1S)-1-methoxyethyl]-3-pyridyl]indol-3-yl]-2,2-dimethyl-propoxy]-tert-butyl-diphenyl-silane (compound A9, 32.0 g, 46.8 mmol) in THF (200 mL) was added tetrabutylammonium fluoride (280.7 mL, 280.7 mmol, 1 M in THF). After being stirred at 50° C. for 12 hrs, the mixture was diluted with water (500 mL) and extracted with EtOAc (300 mL). The combined organic layer was washed with brine (200 mL), dried over anhydrous sodium sulfate, filtered and concentrated under vacuum. The residue was purified by silica gel chromatography (EA/PE: 0-20%) to afford 3-[5-bromo-1-ethyl-(2M)-2-[2-[(1S)-1-methoxyethyl]-3-pyridyl]indol-3-yl]-2,2-dimethyl-propan-1-ol (intermediate A, 7.4 g, faster eluted) as a yellow gum and 3-[5-bromo-1-ethyl-(2P)-2-[2-[(1S)-1-methoxyethyl]-3-pyridyl]indol-3-yl]-2,2-dimethyl-propan-1-ol (compound A10, 5.6 g, slower eluted) as a yellow solid. Intermediate A: ¹H NMR (400 MHz, CDCl₃) δ ppm 8.83 (dd, J=4.8, 2.0 Hz, 1H), 7.90 (d, J=1.6 Hz, 1H), 7.69 (dd, J=7.6, 1.6 Hz, 1H), 7.39-7.32 (m, 2H), 7.26-7.23 (m, 1H), 4.12-4.07 (m, 1H), 4.03-3.95 (m, 1H), 3.92-3.81 (m, 1H), 3.27 (dd, J=24.8 10.4 Hz, 2H), 3.07 (s, 3H), 2.74 (d, J=14.0 Hz, 1H), 2.26 (d, J=14.0 Hz, 1H), 1.60-1.52 (m, 1H), 1.48 (d, J=6.4 Hz, 3H), 1.19 (t, J=7.2 Hz, 3H), 0.77 (s, 6H). MS: calc'd 445 (MH⁺), measured 445.1 (MH⁺).

### X-Ray Crystallographic Analysis of Intermediate A

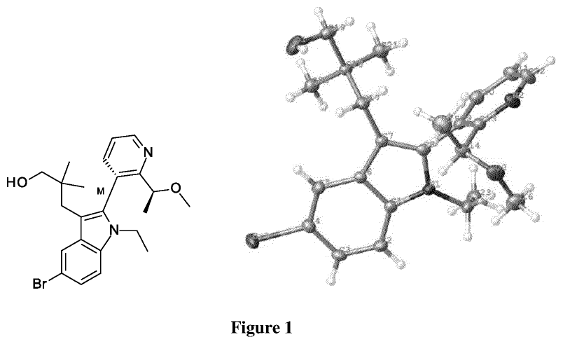

Absolute configuration structure of intermediate A was confirmed by X-ray crystallographic analysis of its single crystal. (FIG. 1)

### Intermediate B

### Methyl (3S)-1-[(2S)-2-(tert-butoxycarbonylamino)-3-[3-(4,4,5,5-tetramethyl-1,3,2-dioxaborolan-2-yl)-5-triisopropylsilyloxy-phenyl]propanoyl]hexahydropyridazine-3-carboxylate

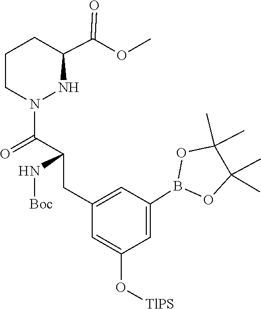

The intermediate B was prepared according to the following scheme:

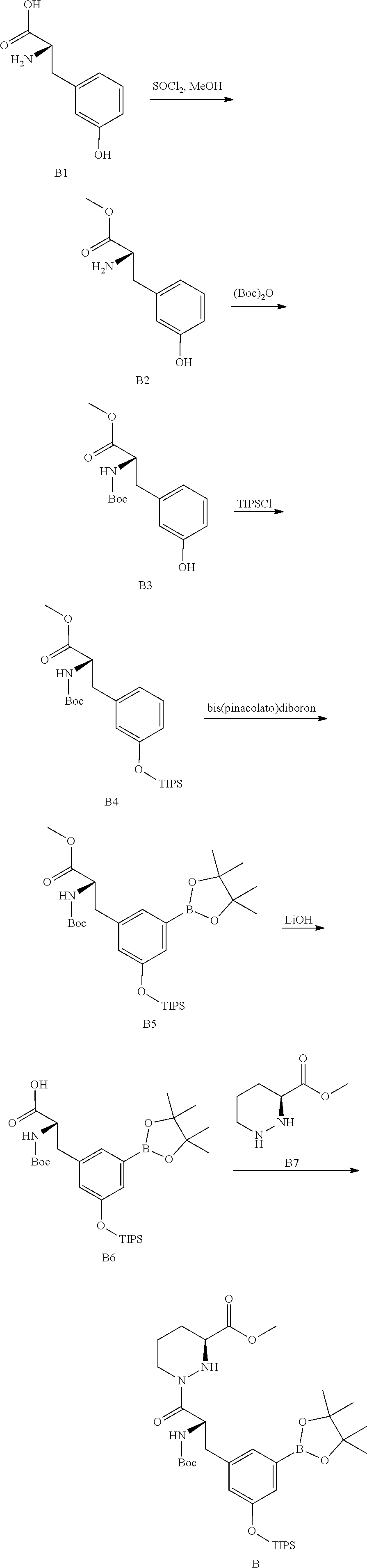

### Step 1: Preparation of methyl (2S)-2-amino-3-(3-hydroxyphenyl)propanoate (compound B2)

To a solution of L-M-tyrosine (compound B1, 5.0 g, 27.6 mmol) in methanol (80 mL) was added thionyl chloride (10 mL, 137.9 mmol). The mixture was stirred at 60° C. for 12 hrs. The reaction mixture was cooled to 20° C. and concentrated in vacuo to afford methyl (2S)-2-amino-3-(3-hydroxyphenyl)propanoate (compound B2, 6.2 g) as a yellow solid. ¹H NMR (400 MHz, CD3OD) δ ppm 7.18 (t, J=8.0 Hz, 1H), 6.78-6.66 (m, 3H), 4.29 (t, J=6.4 Hz, 1H), 3.82 (s, 3H), 3.23-3.05 (m, 2H).

### Step 2: Preparation of methyl (2S)-2-(tert-butoxycarbonylamino)-3-(3-hydroxyphenyl)propanoate (compound B3)

To a solution of methyl (2S)-2-amino-3-(3-hydroxyphenyl)propanoate (compound B2, 32.0 g, 138.1 mmol) in THF (80 mL) and water (20 mL) was added sodium bicarbonate (40.6 g, 483.4 mmol) followed by di-t-butyldicarbonate (33.1 g, 151.9 mmol) at 20° C. The mixture was stirred at 20° C. for 12 hours. The mixture was diluted with water (100 mL) and acidified by 1 M aq. solution of HCl until the pH=5. The mixture was extracted with ethyl acetate (100 mL) for 3 times. The combined organic phase was washed by brine (80 mL), dried over anhydrous sodium sulfate, filtered and the filtrate was concentrated under vacuum to afford methyl (2S)-2-(tert-butoxycarbonylamino)-3-(3-hydroxyphenyl)propanoate (compound B3, 40 g) as colorless gum. MS: calc'd 318 (MNa⁺), measured 318.3 (MNa⁺).

### Step 3: Preparation of methyl (2S)-2-(tert-butoxycarbonylamino)-3-(3-triisopropylsilyloxyphenyl)propanoate (compound B4)

To a solution of methyl (2S)-2-(tert-butoxycarbonylamino)-3-(3-hydroxyphenyl)propanoate (compound B3, 40.0 g, 135.4 mmol) and 1H-imidazole (27.6 g, 406.3 mmol) in DMF (400 mL) was added triisopropylsilyl chloride (39.1 g, 203.1 mmol) dropwise at 0° C. After being stirred for 12 hrs at 25° C., the mixture was diluted with water (250 mL) at 0° C. and extracted with ethyl acetate (200 mL) for 3 times. The combined organic phase was washed by brine (80 mL) for 4 times, dried over anhydrous sodium sulfate, filtered and the filtrate was concentrated under vacuum to give a residue. The residue was purified by silica gel chromatography (EA/PE: 0-20%) to afford methyl (2S)-2-(tert-butoxycarbonylamino)-3-(3-triisopropylsilyloxyphenyl)propanoate (compound B4, 60 g) as yellow oil. MS: calc'd 474 (MNa⁺), measured 474.2 (MNa⁺).

### Step 4: Preparation of methyl (2S)-2-(tert-butoxycarbonylamino)-3-[3-(4,4,5,5-tetramethyl-1,3,2-dioxaborolan-2-yl)-5-triisopropylsilyloxy-phenyl]propanoate (compound B5)

To a solution of methyl (2S)-2-(tert-butoxycarbonylamino)-3-(3-triisopropylsilyloxyphenyl)propanoate (compound B4, 15.0 g, 33.2 mmol), 4,4′-di-tert-butyl-2,2′-bipyridine (2.6 g, 9.9 mmol) and bis(pinacolato)diboron (12.6 g, 49.8 mmol) in hexane (200 mL) was added [Ir(OMe)(COD)]₂ (2.2 g, 3.3 mmol). The mixture was degassed and purged with N₂ for 3 times. The resulting mixture was stirred at 70° C. for 12 hrs. Then the reaction mixture was cooled to 20° C., diluted with petroleum ether (100 mL) and filtered. The filtrate was concentrated in vacuo to give a residue, which was purified by silica gel chromatography (EA/PE: 0-20%) to afford methyl (2S)-2-(tert-butoxycarbonylamino)-3-[3-(4,4,5,5-tetramethyl-1,3,2-dioxaborolan-2-yl)-5-triisopropylsilyloxy-phenyl]propanoate (compound B5, 21 g) as yellow oil. MS: calc'd 600 (MNa⁺), measured 600.3 (MNa⁺).

### Step 5: Preparation of (2S)-2-(tert-butoxycarbonylamino)-3-[3-(4,4,5,5-tetramethyl-1,3,2-dioxaborolan-2-yl)-5-triisopropylsilyloxy-phenyl]propanoic acid (compound B6)

To a solution of methyl (2S)-2-(tert-butoxycarbonylamino)-3-[3-(4,4,5,5-tetramethyl-1,3,2-dioxaborolan-2-yl)-5-triisopropylsilyloxy-phenyl]propanoate (compound B5, 40.0 g, 69.2 mmol) in methanol (300 mL) was added a solution of lithium hydroxide (3.2 mL, 346.2 mmol) in water (100 mL). After being stirred at 20° C. for 1 hr, the reaction mixture was diluted with water (200 mL) and MeOH was removed under vacuum. The resulting mixture was acidified by 1 M aq. solution of HCl until the pH=5. The resulting mixture was extracted with EtOAc (250 mL) for 3 times. The organic phase was washed with brine (150 mL), dried over anhydrous sodium sulfate, filtered and the filtrate was concentrated in vacuo to afford (2S)-2-(tert-butoxycarbonylamino)-3-[3-(4,4,5,5-tetramethyl-1,3,2-dioxaborolan-2-yl)-5-triisopropylsilyloxy-phenyl]propanoic acid (compound B6, 33 g) as a white solid. MS: calc'd 586 (MNa⁺), measured 586.3 (MNa⁺).

### Step 6: Preparation of methyl (3S)-1-[(2S)-2-(tert-butoxycarbonylamino)-3-[3-(4,4,5,5-tetramethyl-1,3,2-dioxaborolan-2-yl)-5-triisopropylsilyloxy-phenyl]propanoyl]hexahydropyridazine-3-carboxylate (Intermediate B)

To a solution of (2S)-2-(tert-butoxycarbonylamino)-3-[3-(4,4,5,5-tetramethyl-1,3,2-dioxaborolan-2-yl)-5-triisopropylsilyloxy-phenyl]propanoic acid (compound B6, 8.0 g, 14.1 mmol) and O-(7-azabenzotriazol-1-yl)-N,N,N′,N′-tetramethyluronium hexafluorophosphate (5.6 g, 14.9 mmol) in DMF (100 mL) was added N,N-diisopropylethylamine (6.4 g, 49.6 mmol). The mixture was stirred at 0° C. for 10 min. Then methyl (3S)-hexahydropyridazine-3-carboxylate hydrochloride salt (compound B7, 2.6 g, 14.9 mmol) was added. The resulting mixture was stirred at 20° C. for 1.5 hrs. The mixture was diluted with water (200 mL) and extracted with EtOAc (100 mL) for 2 times. The combined organic phase was washed with brine, dried over anhydrous sodium sulfate, filtrated and the filtrate was concentrated under vacuum. The residue was purified by silica gel chromatography (EA/PE: 0-20%) to afford methyl (3S)-1-[(2S)-2-(tert-butoxycarbonylamino)-3-[3-(4,4,5,5-tetramethyl-1,3,2-dioxaborolan-2-yl)-5-triisopropylsilyloxy-phenyl]propanoyl]hexahydropyridazine-3-carboxylate (intermediate B, 7.8 g) as yellow oil. MS: calc'd 690 (MH⁺), measured 690.4 (MH⁺).

### Intermediate C

### (8S,14S)-8-amino-22-ethyl-4-hydroxy-(21M)-21-[2-[(1S)-1-methoxyethyl]-3-pyridyl]-18,18-dimethyl-16-oxa-10,22,28-triazapentacyclo[18.5.2.12,6.110,14.023,27]nonacosa-1(26),2,4,6(29),20,23(27),24-heptaene-9,15-dione

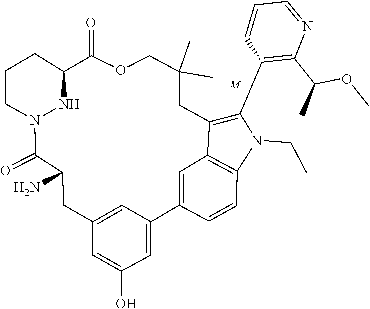

The intermediate C was prepared according to the following scheme:

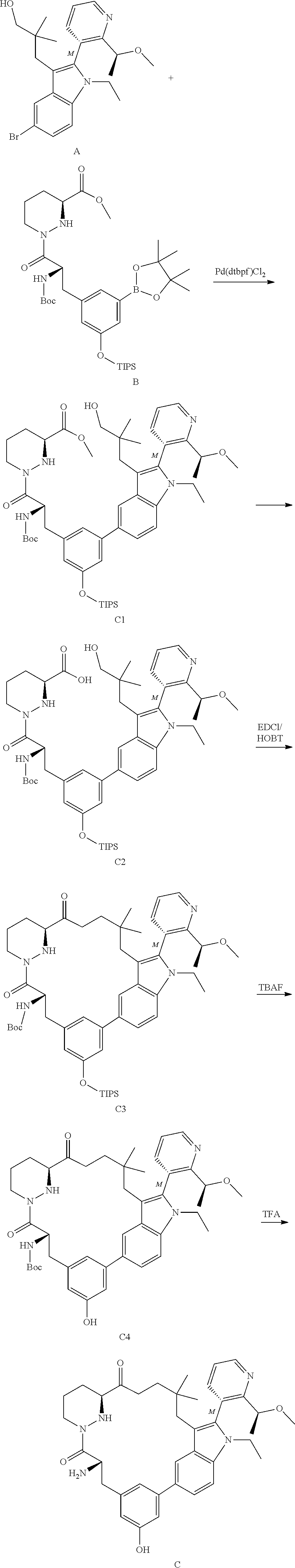

### Step 1: Preparation of methyl (3S)-1-[(2S)-2-(tert-butoxycarbonylamino)-3-[3-[1-ethyl-3-(3-hydroxy-2,2-dimethyl-propyl)-(2M)-2-[2-[(1S)-1-methoxyethyl]-3-pyridyl]indol-5-yl]-5-triisopropylsilyloxy-phenyl]propanoyl]hexahydropyridazine-3-carboxylate (compound C1)

To a mixture of methyl (3S)-1-[(2S)-2-(tert-butoxycarbonylamino)-3-[3-(4,4,5,5-tetramethyl-1,3,2-dioxaborolan-2-yl)-5-triisopropylsilyloxy-phenyl]propanoyl]hexahydropyridazine-3-carboxylate (intermediate B, 1.1 g, 1.6 mmol) and 3-[5-bromo-1-ethyl-(2M)-2-[2-[(1S)-1-methoxyethyl]-3-pyridyl]indol-3-yl]-2,2-dimethyl-propan-1-ol (intermediate A, 356.9 mg, 3.3 mmol) in 1,4-dioxane (12 mL) and water (1.2 mL) was added Pd(dtbpf)Cl₂ (87.7 mg, 0.13 mmol). The mixture was degassed and purged N₂ for 3 times. The resulting mixture was stirred at 85° C. for 12 hrs. The reaction mixture was cooled to 20° C. and filtered. The filtrate was concentrated under vacuum. The residue was purified by silica gel chromatography (EA/PE: 0-50%) to afford methyl (3S)-1-[(2S)-2-(tert-butoxycarbonylamino)-3-[3-[1-ethyl-3-(3-hydroxy-2,2-dimethyl-propyl)-(2M)-2-[2-[(1S)-1-methoxyethyl]-3-pyridyl]indol-5-yl]-5-triisopropylsilyloxy-phenyl]propanoyl]hexahydropyridazine-3-carboxylate (compound C1, 750 mg) as a yellow oil. MS: calc'd 928 (MH⁺), measured 928.3 (MH⁺).

### Step 2: Preparation of (3S)-1-[(2S)-2-(tert-butoxycarbonylamino)-3-[3-[1-ethyl-3-(3-hydroxy-2,2-dimethyl-propyl)-(2M)-2-[2-[(1S)-1-methoxyethyl]-3-pyridyl]indol-5-yl]-5-triisopropylsilyloxy-phenyl]propanoyl]hexahydropyridazine-3-carboxylic acid (compound C2)

To a solution of methyl (3S)-1-[(2S)-2-(tert-butoxycarbonylamino)-3-[3-[1-ethyl-3-(3-hydroxy-2,2-dimethyl-propyl)-(2M)-2-[2-[(1S)-1-methoxyethyl]-3-pyridyl]indol-5-yl]-5-triisopropylsilyloxy-phenyl]propanoyl]hexahydropyridazine-3-carboxylate (compound C1, 750.0 mg, 0.7 mmol) in DCE (12 mL) was added trimethyltin hydroxide (519.5 mg, 2.8 mmol). The mixture was stirred at 60° C. for 12 hrs. LCMS showed starting material was consumed and desired product was formed. The mixture was added into water (40 mL) and extracted with ethyl acetate (50 mL) for 3 times. The combined organic phase was washed by brine (40 mL), dried over anhydrous sodium sulfate, filtered and concentrated under vacuum to afford (3S)-1-[(2S)-2-(tert-butoxycarbonylamino)-3-[3-[1-ethyl-3-(3-hydroxy-2,2-dimethyl-propyl)-(2M)-2-[2-[(1S)-1-methoxyethyl]-3-pyridyl]indol-5-yl]-5-triisopropylsilyloxy-phenyl]propanoyl]hexahydropyridazine-3-carboxylic acid (compound C2, 650 mg) as a yellow solid. MS: calc'd 914 (MH⁺), measured 914.5 (MH⁺).

### Step 3: Preparation of tert-butyl N-[(8S,14S)-22-ethyl-(21M)-21-[2-[(1S)-1-methoxyethyl]-3-pyridyl]-18,18-dimethyl-9,15-dioxo-4-triisopropylsilyloxy-16-oxa-10,22,28-triazapentacyclo[18.5.2.12,6.110,14.023,27]nonacosa-1(26),2,4,6(29),20,23(27),24-heptaen-8-yl]carbamate (compound C3)

To a solution of (3S)-1-[(2S)-2-(tert-butoxycarbonylamino)-3-[3-[1-ethyl-3-(3-hydroxy-2,2-dimethyl-propyl)-(2M)-2-[2-[(1S)-1-methoxyethyl]-3-pyridyl]indol-5-yl]-5-triisopropylsilyloxy-phenyl]propanoyl]hexahydropyridazine-3-carboxylic acid (compound C2, 650.0 mg, 0.7 mmol) in DCM (10 mL) was added DIEA (1.4 g, 11.3 mmol), EDCI (1.9 g, 10.6 mmol) followed by HOBT (240.1 mg, 1.8 mmol) at 0° C. The mixture was stirred at 20° C. for 12 hrs. The mixture was poured into water (40 mL) and exacted with EtOAc (30 mL) for 3 times. The combined organic phase was washed by brine (40 mL), dried over sodium sulfate, filtered and concentrated under vacuum. The residue was purified by silica gel chromatography (EA/PE: 0-50%) to afford tert-butyl N-[(8S,14S)-22-ethyl-(21M)-21-[2-[(1S)-1-methoxyethyl]-3-pyridyl]-18,18-dimethyl-9,15-dioxo-4-triisopropylsilyloxy-16-oxa-10,22,28-triazapentacyclo[18.5.2.12,6.110,14.023,27]nonacosa-1(26),2,4,6(29),20,23(27),24-heptaen-8-yl]carbamate (compound C3, 530 mg) as yellow oil. MS: calc'd 896 (MH⁺), measured 896.2 (MH⁺).

### Step 4: Preparation of tert-butyl N-[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamate (compound C4)

To a solution of tert-butyl N-[(8S,14S)-22-ethyl-(21M)-21-[2-[(1S)-1-methoxyethyl]-3-pyridyl]-18,18-dimethyl-9,15-dioxo-4-triisopropylsilyloxy-16-oxa-10,22,28-triazapentacyclo[18.5.2.12,6.110,14.023,27]nonacosa-1(26),2,4,6(29),20,23(27),24-heptaen-8-yl]carbamate (compound C3, 480.0 mg, 0.5 mmol) in THF (5 mL) was added TBAF (0.6 mL, 0.6 mmol, 1.0 M in THF) at 0° C. After being stirred at 0° C. for 0.5 hr, the reaction mixture was diluted with water (30 mL) and exacted with EtOAc (40 mL) for 3 times. The combined organic phase was washed by brine (50 mL), dried over sodium sulfate, filtered and concentrated under vacuum to afford tert-butyl N-[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamate (compound C4, 390 mg) as colorless gum. MS: calc'd 740 (MH⁺), measured 740.2 (MH⁺).

### Step 5: Preparation of (8S,14S)-8-amino-22-ethyl-4-hydroxy-(21M)-21-[2-[(1S)-1-methoxyethyl]-3-pyridyl]-18,18-dimethyl-16-oxa-10,22,28-triazapentacyclo[18.5.2.12,6.110,14.023,27]nonacosa-1(26),2,4,6(29),20,23(27),24-heptaene-9,15-dione (intermediate C)

To a solution of tert-butyl N-[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamate (compound C4, 430.0 mg, 0.6 mmol) in DCM (4 mL) was added TFA (0.8 mL). After being stirred at 20° C. for 1 h, the reaction mixture was diluted with saturated aqueous solution of NaHCO₃ (40 mL) and extracted with EtOAc (60 mL) for 3 times. The combined organic phase was washed by brine (30 mL), dried over sodium sulfate, filtered and concentrated under vacuum to afford (8S,14S)-8-amino-22-ethyl-4-hydroxy-(21M)-21-[2-[(1S)-1-methoxyethyl]-3-pyridyl]-18,18-dimethyl-16-oxa-10,22,28-triazapentacyclo[18.5.2.12,6.110,14.023,27]nonacosa-1(26),2,4,6(29),20,23(27),24-heptaene-9,15-dione (intermediate C, 330 mg) as yellow solid. MS: calc'd 640 (MH⁺), measured 640.3 (MH⁺). ¹H NMR (400 MHz, CD₃OD) δ ppm 8.72-8.71 (m, 1H), 7.89 (d, J=7.6 Hz, 1H), 7.81 (s, 1H), 7.55-7.44 (m, 3H), 7.11 (s, 1H), 6.92 (s, 1H), 6.60 (s, 1H), 4.75 (t, J=7.2 Hz, 1H), 4.60 (br. s, 1H), 4.41 (d, J=12.8 Hz, 1H), 4.35-4.20 (m, 2H), 4.14-4.05 (m, 1H), 3.62 (d, J=10.8 Hz, 1H), 3.49 (d, J=10.8 Hz, 1H), 3.26 (s, 2H), 3.13-3.00 (m, 1H), 2.89-2.84 (m, 1H), 2.81-2.78 (m, 3H), 2.66-2.62 (m, 1H), 2.05-2.00 (m, 1H), 1.82-1.80 (m, 1H), 1.51-1.46 (m, 5H), 1.02 (t, J=6.8 Hz, 3H), 0.86-0.82 (m, 3H), 0.59-0.56 (m, 3H).

### Intermediate D

### Benzyl 4-[(5M)-5-[5-bromo-1-ethyl-3-(3-hydroxy-2,2-dimethyl-propyl)indol-2-yl]-6-[(1S)-1-methoxyethyl]-3-pyridyl]piperazine-1-carboxylate

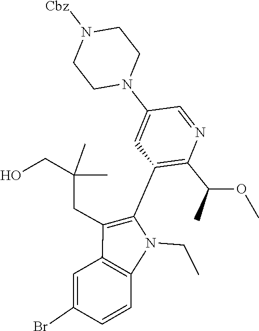

The title compound was prepared in analogy to the preparation of Intermediate A by using benzyl 4-[5-bromo-6-[(1S)-1-methoxyethyl]-3-pyridyl]piperazine-1-carboxylate (compound D5) instead of 3-bromo-2-[(1S)-1-methoxyethyl]pyridine (compound A11).

The compound D5 was prepared according to the following scheme:

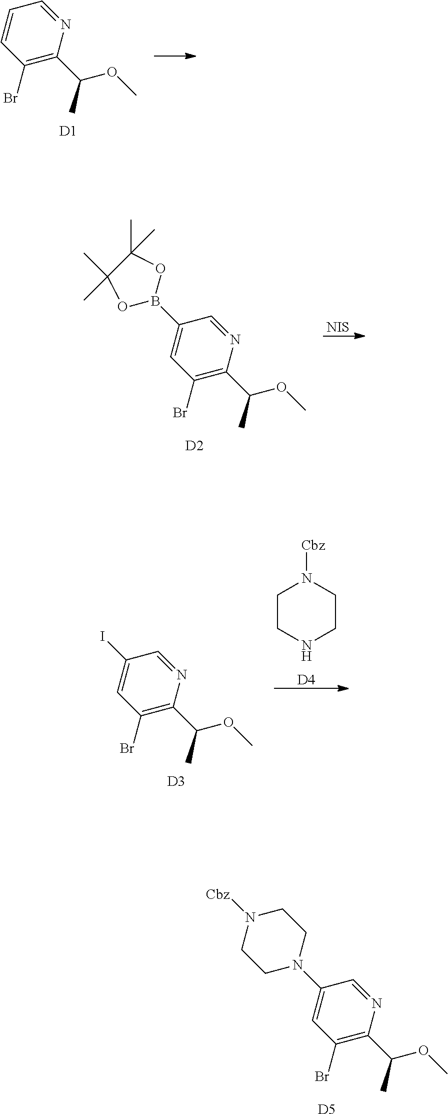

### Step 1: Preparation of 3-bromo-2-[(1S)-1-methoxyethyl]-5-(4,4,5,5-tetramethyl-1,3,2-dioxaborolan-2-yl)pyridine (compound D2)

To a solution of 3-bromo-2-[(1S)-1-methoxyethyl]pyridine (compound D1, 2.0 g, 9.26 mmol,) and bis(pinacolato)diboron (3.5 g, 13.9 mmol) in THF (30 mL) were added 4,4′-di-tert-butyl-2,2′-bipyridin (372.7 mg, 1.39 mmol) and [Ir(OMe)(COD)]₂ (306.3 mg, 0.460 mmol). The mixture was stirred at 75° C. for 16 hours under N₂ protection. The mixture was filtrated and the filtrate was concentrated in vacuo. The residue was purified by silica gel chromatography (EA/PE: 0-20%) to afford 3-bromo-2-[(1S)-1-methoxyethyl]-5-(4,4,5,5-tetramethyl-1,3,2-dioxaborolan-2-yl)pyridine (compound D2, 2.4 g) as yellow oil. ¹H NMR (400 MHz, CDCl3) δ ppm 8.91 (d, J=1.4 Hz, 1H), 8.21 (d, J=1.4 Hz, 1H), 4.95 (q, J=6.5 Hz, 1H), 3.30 (s, 3H), 1.49 (d, J=6.5 Hz, 3H), 1.35 (s, 12H).

### Step 2: Preparation of 3-bromo-5-iodo-2-[(1S)-1-methoxyethyl]pyridine (compound D3)

To a solution of 3-bromo-2-[(1S)-1-methoxyethyl]-5-(4,4,5,5-tetramethyl-1,3,2-dioxaborolan-2-yl)pyridine (compound D2, 2.5 g, 7.3 mmol) in ACN (40 mL) was added N-iodosuccinimide (4.1 g, 18.27 mmol). The mixture was stirred at 90° C. for 40 h under N₂ protection. The mixture was quenched with saturated solution of Na₂SO₃ (40 mL) and extracted with EtOAc (30 mL, twice). The combined organic layer was washed with brine (50 mL), filtered and the filtrate was concentrated in vacuum. The residue was purified by silica gel chromatography (EA/PE: 0-20%) to afford 3-bromo-5-iodo-2-[(1S)-1-methoxyethyl]pyridine (compound D3, 660 mg) as yellow oil. MS calc'd 342 (MH⁺), measured 341.8 (MH⁺).

### Step 3: Preparation of benzyl 4-[5-bromo-6-[(1S)-1-methoxyethyl]-3-pyridyl]piperazine-1-carboxylate (compound D5)

To a solution of 3-bromo-5-iodo-2-[(1S)-1-methoxyethyl]pyridine (compound D3, 660 mg, 1.9 mmol) and 1-Cbz-piperazine (compound D4, 425.1 mg, 1.9 mmol) in toluene (10 mL) were added cesium carbonate (1.6 g, 4.83 mmol), (R)-BINAP (60.1 mg, 0.1 mmol) and palladium (II) acetate (43.3 mg, 0.19 mmol). The mixture was stirred at 100° C. for 12 hours under N₂ protection. The mixture was filtered and the filtrate was concentrated under vacuum. The residue was purified by silica gel chromatography (EA/PE: 0-50%) to afford benzyl 4-[5-bromo-6-[(1S)-1-methoxyethyl]-3-pyridyl]piperazine-1-carboxylate (compound D5, 740 mg) as a yellow solid. MS calc'd 434.1 (MH⁺), measured 434.1 (MH⁺).

### Intermediate E

### Methyl (3S)-1-[(2S)-3-(4-bromothiazol-2-yl)-2-(tert-butoxycarbonylamino)propanoyl]hexahydropyridazine-3-carboxylate

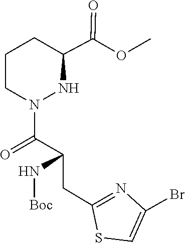 [SMILES: COC(=O)[C@@H]1CCCN(C(=O)[C@H](Cc2nc(Br)cs2)NC(=O)OC(C)(C)C)N1]

The intermediate E was prepared according to the following scheme:

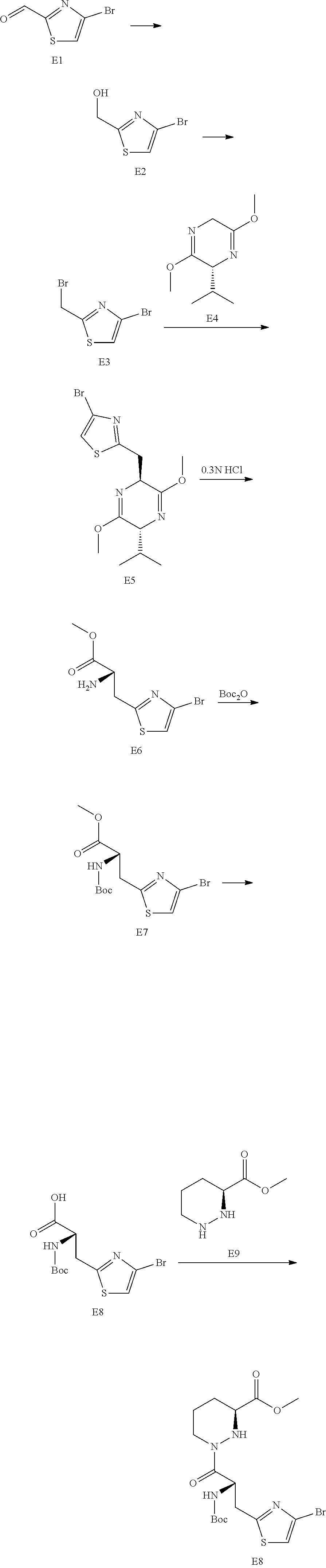

### Step 1: Preparation of (4-bromothiazol-2-yl)methanol (compound E2)

To a solution of 4-bromothiazole-2-carboxaldehyde (6.0 g, 31.25 mmol) in methanol (70 mL) was added sodium borohydride (1.77 g, 46.87 mmol) at 0° C. The mixture was stirred at 25° C. for 1 hour. The reaction mixture was quenched with water (300 mL) at 0° C. and extracted by ethyl acetate (200 mL, three times). The combined organic phase was washed by brine (150 mL, twice), dried over anhydrous sodium sulfate, filtered and the filtrate was concentrated under vacuum to afford (4-bromothiazol-2-yl)methanol (compound E2, 6 g) as colorless oil.

### Step 2: Preparation of 4-bromo-2-(bromomethyl)thiazole (compound E3)

To a solution of (4-bromothiazol-2-yl)methanol (compound E2, 6.0 g, 30.92 mmol) in DCM (80 mL) was added CBr₄ (15.38 g, 46.38 mmol) and triphenylphosphine (12.16 g, 46.38 mmol) at 0° C. After being stirred at 25° C. for 1 hour, the mixture was filtered and the filtrate was concentrated under vacuum. The residue was purified by silica gel column, eluted with ethyl acetate in petroleum ether=0˜10% to afford (4-bromothiazol-2-yl)methanol (compound E3, 6.0 g) as yellow oil. MS calc'd 255.9 (MH⁺). measured 255.9 (MH⁺.

### Step 3: Preparation of 4-bromo-2-[[(2S,5R)-5-isopropyl-3,6-dimethoxy-2,5-dihydropyrazin-2-yl]methyl]thiazole (compound E5)

To a mixture of (R)-2,5-dihydro-3,6-dimethoxy-2-isopropylpyrazine (compound E4, 4.32 g, 23.45 mmol) in THF (60 mL) was added n-butyllithium (10 mL, 25.22 mmol, 2.5 M) at −78° C. slowly. After addition, the mixture was stirred for 0.5 hour at −78° C. 4-bromo-2-(bromomethyl)thiazole (compound E3, 5.4 g, 21.02 mmol) was added into above mixture at −78° C. which was stirred for another 1 hour. The mixture was quenched with saturated aqueous solution of NH₄Cl (100 mL) and extracted with EtOAc (100 mL, twice). The combined organic layer was washed with brine (150 mL), dried over anhydrous sodium sulfate, filtered and the filtrate was concentrated. The residue was purified by reversed-phase chromatography, eluted with ACN in H₂O (0.01% FA)=0˜60% to afford 4-bromo-2-[[(2S,5R)-5-isopropyl-3,6-dimethoxy-2,5-dihydropyrazin-2-yl]methyl]thiazole (compound E5, 3.6 g) as yellow oil. MS calc'd 360 (MH⁺), measured 359.9 (MH⁺).

### Step 4: Preparation of methyl (2S)-2-amino-3-(4-bromothiazol-2-yl)propanoate (compound E6)

To a solution of 4-bromo-2-[[(2S,5R)-5-isopropyl-3,6-dimethoxy-2,5-dihydropyrazin-2-yl]methyl]thiazole (compound E5, 3.6 g, 10 mmol) in ACN (20 mL) was added hydrochloric acid (66.62 mL, 0.3 M). The mixture was stirred at 25° C. for 2 hours. The mixture was basified by saturated aqueous solution of NaHCO₃ until pH=8. The mixture was extracted with EtOAc (80 mL, six times). The combined organic layer was dried over anhydrous sodium sulfate, filtered and the filtrate was concentrated under vacuum to afford methyl (2S)-2-amino-3-(4-bromothiazol-2-yl)propanoate (compound E6, 3.1 g) as yellow oil. MS calc'd 264.9 (MH⁺), measured 264.9 (MH⁺).

### Step 5: Preparation of methyl (2S)-3-(4-bromothiazol-2-yl)-2-(tert-butoxycarbonylamino)propanoate (compound E7)

To a solution of methyl (2S)-2-amino-3-(4-bromothiazol-2-yl)propanoate (E6, 3.1 g, 11.69 mmol) in DCM (40 mL) were added triethylamine (2.96, 29.23 mmol) and (Boc)₂₀ (3.83 g, 17.54 mmol). The mixture was stirred at 30° C. for 12 hours. The mixture was concentrated under vacuum. The residue was purified by silica gel column, eluted with ethyl acetate in petroleum ether=0˜30% to afford methyl (2S)-3-(4-bromothiazol-2-yl)-2-(tert-butoxycarbonylamino)propanoate (compound E7, 3.2 g) as yellow oil. MS calc'd 387 (MNa⁺), measured 386.9 (MNa⁺).

### Step 6: Preparation of (2S)-3-(4-bromothiazol-2-yl)-2-(tert-butoxycarbonylamino)propanoic acid (compound E8)

To a solution of methyl (2S)-3-(4-bromothiazol-2-yl)-2-(tert-butoxycarbonylamino)propanoate (compound E7, 3.2 g, 8.76 mmol) in THF (30 mL) and methanol (2 mL) and water (10 mL) was added lithium hydroxide (0.41 mL, 43.81 mmol). The mixture was stirred at 25° C. for 1 hour. The mixture was acidified by 1 M aqueous solution of HCl until pH=5. The mixture was extracted with EtOAc (40 mL, twice). The combined organic layer was washed with brine (100 mL), dried over anhydrous sodium sulfate, filtered and the filtrate was concentrated under vacuum to afford (2S)-3-(4-bromothiazol-2-yl)-2-(tert-butoxycarbonylamino)propanoic acid (compound E8, 3.1 g) as yellow oil. MS calc'd 373 (MNa⁺), measured 372.9 (MNa⁺).

### Step 7: Preparation of methyl (3S)-1-[(2S)-3-(4-bromothiazol-2-yl)-2-(tert-butoxycarbonylamino)propanoyl]hexahydropyridazine-3-carboxylate (E)

To a solution of (2S)-3-(4-bromothiazol-2-yl)-2-(tert-butoxycarbonylamino)propanoic acid (compound E8, 3.1 g, 8.83 mmol) in DCM (50 mL) was added methyl (3S)-hexahydropyridazine-3-carboxylate;hydrochloride (compound E9, 2.39 g, 13.24 mmol), EDCI (3.38 g, 17.65 mmol), 1-Hydroxybenzotriazole (238.53 mg, 1.77 mmol) and NMM (9.92 mL, 88.26 mmol) at 0° C. The mixture was stirred at 25° C. for 1 hour. The mixture was diluted with water (60 mL) and extracted with EtOAc (60 mL, three times). The combined organic layer was washed with brine (100 mL), dried over anhydrous sodium sulfate, filtered and the filtrate was concentrated under vacuum. The residue was purified by silica gel column and eluted with ethyl acetate in petroleum ether=10˜30% to afford methyl (3S)-1-[(2S)-3-(4-bromothiazol-2-yl)-2-(tert-butoxycarbonylamino)propanoyl]hexahydropyridazine-3-carboxylate (intermediate E, 2.4 g). MS calc'd 477 (MH⁺), measured 476.9 (MH⁺).

### Intermediate F

### (7S,13S)-7-amino-21-ethyl-(20M)-20-[2-[(1S)-1-methoxyethyl]-5-(4-methylpiperazin-1-yl)-3-pyridyl]-17,17-dimethyl-15-oxa-4-thia-9,21,27,28-tetrazapentacyclo[17.5.2.12,5.19,13.022,26]octacosa-1(25),2,5(28),19,22(26),23-hexaene-8,14-dione

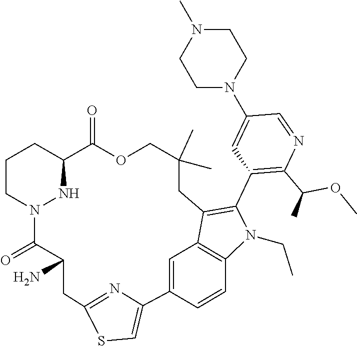

The compound intermediate F was prepared according to the following scheme:

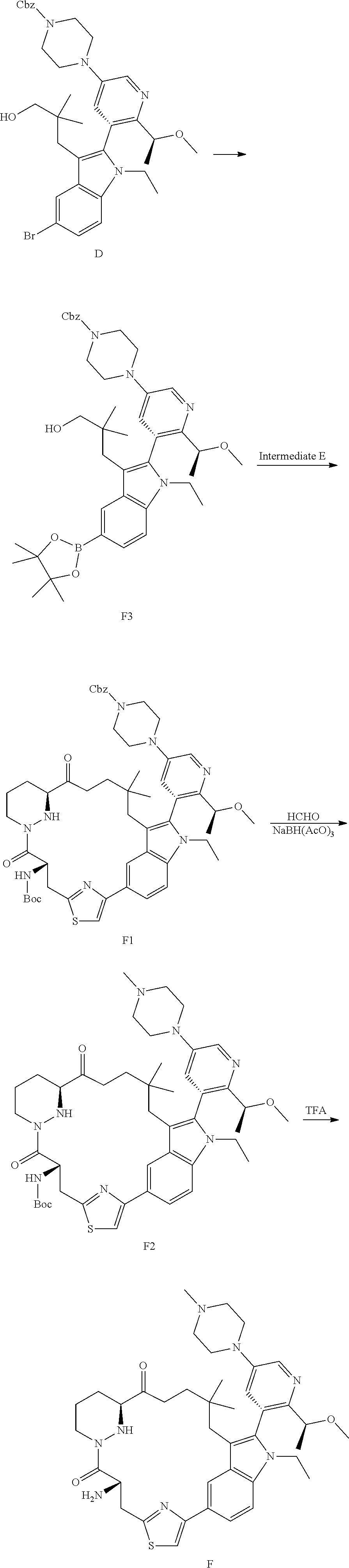

### Step 1: Preparation of benzyl 4-[(5M)-5-[1-ethyl-3-(3-hydroxy-2,2-dimethyl-propyl)-5-(4,4,5,5-tetramethyl-1,3,2-dioxaborolan-2-yl)indol-2-yl]-6-[(1S)-1-methoxyethyl]-3-pyridyl]piperazine-1-carboxylate (compound F3)

To a solution of benzyl 4-[(5M)-5-[5-bromo-1-ethyl-3-(3-hydroxy-2,2-dimethyl-propyl)indol-2-yl]-6-[(1S)-1-methoxyethyl]-3-pyridyl]piperazine-1-carboxylate (intermediate D, 110.0 mg, 0.17 mmol) and bis(pinacolato)diboron (46.3 mg, 0.18 mmol) in toluene (4 mL) was added KOAc (40.67 mg, 0.4 mmol) and [1,1′-bis(diphenylphosphino)ferrocene]dichloropalladium(II) (12.13 mg, 0.02 mmol). After being stirred at 90° C. for 12 hours under N₂ protection, the mixture was concentrated under vacuum. The residue was purified by silica gel column, eluted with ethyl acetate in petroleum ether=30˜60% to afford benzyl 4-[(5M)-5-[1-ethyl-3-(3-hydroxy-2,2-dimethyl-propyl)-5-(4,4,5,5-tetramethyl-1,3,2-dioxaborolan-2-yl)indol-2-yl]-6-[(1S)-1-methoxyethyl]-3-pyridyl]piperazine-1-carboxylate (compound F3, 100 mg) as yellow oil. MS calc'd 711 (MH⁺), measured 711.1 (MH⁺).

### Step 2: Preparation of benzyl 4-[(5M)-5-[(7S,13S)-7-(tert-butoxycarbonylamino)-21-ethyl-17,17-dimethyl-8,14-dioxo-15-oxa-4-thia-9,21,27,28-tetrazapentacyclo[17.5.2.12,5.19,13.022,26]octacosa-1(25),2,5(28),19,22(26),23-hexaen-20-yl]-6-[(1S)-1-methoxyethyl]-3-pyridyl]piperazine-1-carboxylate (compound F1)

Compound F1 was prepared in analogy to the preparation of Intermediate C by using 4-[(5M)-5-[1-ethyl-3-(3-hydroxy-2,2-dimethyl-propyl)-5-(4,4,5,5-tetramethyl-1,3,2-dioxaborolan-2-yl)indol-2-yl]-6-[(1S)-1-methoxyethyl]-3-pyridyl]piperazine-1-carboxylate (compound F3) and methyl (3S)-1-[(2S)-3-(4-bromothiazol-2-yl)-2-(tert-butoxycarbonylamino)propanoyl]hexahydropyridazine-3-carboxylate (Intermediate E) instead of 3-[(5M)-5-bromo-1-ethyl-2-[2-[(1S)-1-methoxyethyl]-3-pyridyl]indol-3-yl]-2,2-dimethyl-propan-1-ol (intermediate A) methyl (3S)-1-[(2S)-2-(tert-butoxycarbonylamino)-3-[3-(4,4,5,5-tetramethyl-1,3,2-dioxaborolan-2-yl)-5-triisopropylsilyloxy-phenyl]propanoyl]hexahydropyridazine-3-carboxylate (intermediate B).

### Step 3: Preparation of tert-butyl N-[(7S,13S)-21-ethyl-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.12,5.19,13.022,26]octacosa-1(25),2,5(28),19,22(26),23-hexaen-7-yl]carbamate (compound F2)

To a solution of benzyl 4-[(5M)-5-[(7S,13S)-7-(tert-butoxycarbonylamino)-21-ethyl-17,17-dimethyl-8,14-dioxo-15-oxa-4-thia-9,21,27,28-tetrazapentacyclo[17.5.2.12,5.19,13.022,26]octacosa-1(25),2,5(28),19,22(26),23-hexaen-20-yl]-6-[(1S)-1-methoxyethyl]-3-pyridyl]piperazine-1-carboxylate (compound F1, 35.0 mg, 0.04 mmol) in methanol (5 mL) was added Pd(OH)₂/C (20 mg). The mixture was degassed and purged with H₂ three times. After being stirred at 25° C. for 3 hours under H₂ balloon, the mixture was filtered and the filtrate was concentrated in vacuo to afford intermediate (20 mg) as a white solid. To a solution of this intermediate (20.0 mg) in methanol (1 mL) was added acetic acid (5.04 mg, 0.08 mmol). After being stirred at 25° C. for 15 min, the mixture was added formaldehyde (5 mg, 0.06 mmol) and NaBH₃CN (2.11 mg, 0.03 mmol), then stirred for another 45 min. The mixture was filtered and the filtrate was concentrated under vacuum. The residue was purified by reversed phase chromatography to afford tert-butyl N-[(7S,13S)-21-ethyl-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.12,5.19,13.022,26]octacosa-1(25),2,5(28),19,22(26),23-hexaen-7-yl]carbamate (compound F2, 17 mg). MS calc'd 829 (MH⁺), measured 829.1 (MH⁺).

### Step 4: Preparation of (7S,13S)-7-amino-21-ethyl-(20M)-20-[2-[(1S)-1-methoxyethyl]-5-(4-methylpiperazin-1-yl)-3-pyridyl]-17,17-dimethyl-15-oxa-4-thia-9,21,27,28-tetrazapentacyclo[17.5.2.12,5.19,13.022,26]octacosa-1(25),2,5(28),19,22(26),23-hexaene-8,14-dione (intermediate F)

To a mixture of tert-butyl N-[(7S,13S)-21-ethyl-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.12,5.19,13.022,26]octacosa-1(25),2,5(28),19,22(26),23-hexaen-7-yl]carbamate (compound F2, 17.0 mg, 0.02 mmol) in DCM (5 mL) was added TFA (1.0 mL). After being stirred at 30° C. for 1 hour, the mixture was concentrated under vacuum and diluted with saturated NaHCO₃ solution (10 mL), extracted with EtOAc. The combined organic layer was washed with brine (15 mL), filtered and concentrated under vacuum to afford (7S,13S)-7-amino-21-ethyl-(20M)-20-[2-[(1S)-1-methoxyethyl]-5-(4-methylpiperazin-1-yl)-3-pyridyl]-17,17-dimethyl-15-oxa-4-thia-9,21,27,28-tetrazapentacyclo[17.5.2.12,5.19,13.022,26]octacosa-1(25),2,5(28),19,22(26),23-hexaene-8,14-dione (intermediate F, 10 mg) as yellow oil. MS calc'd 729 (MH⁺), measured 729.2 (MH⁺).

### Intermediate G

### (8S,14S)-8-amino-22-ethyl-4-hydroxy-(21M)-21-[2-[(1S)-1-methoxyethyl]-5-(4-methylpiperazin-1-yl)-3-pyridyl]-18,18-dimethyl-16-oxa-10,22,28-triazapentacyclo[18.5.2.12,6.110,14.023,27]nonacosa-1(26),2,4,6(29),20,23(27),24-heptaene-9,15-dione

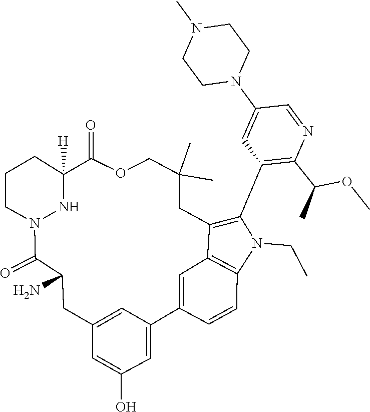

The compound was prepared according to the following scheme

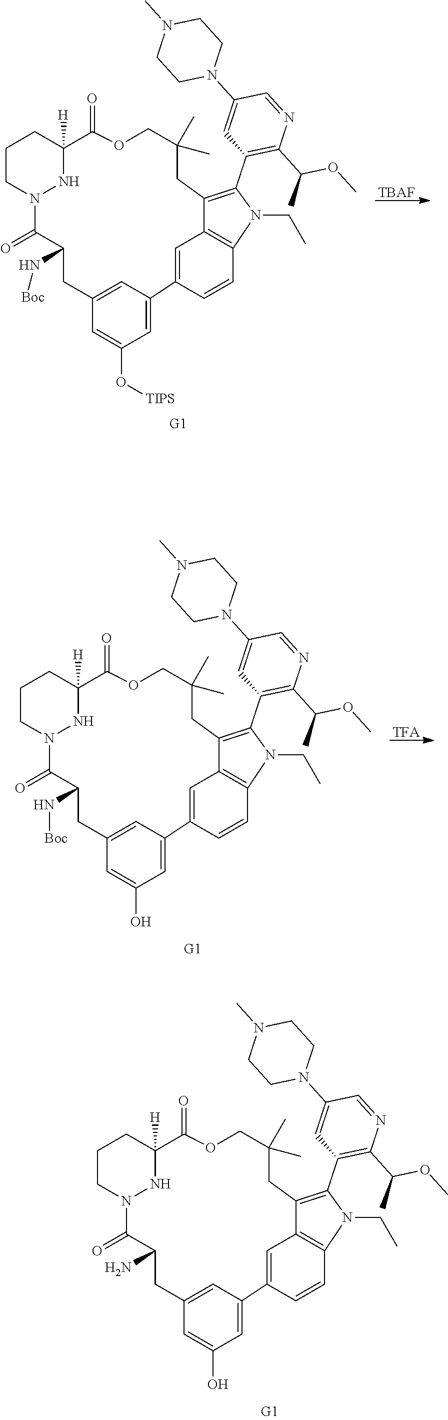

### Step 1: Preparation of tert-butyl N-[(8S,14S)-22-ethyl-4-hydroxy-(21M)-21-[2-[(1S)-1-methoxyethyl]-5-(4-methylpiperazin-1-yl)-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamate (compound G2)

To a solution of tert-butyl N-[(8S,14S)-22-ethyl-(21M)-21-[2-[(1S)-1-methoxyethyl]-5-(4-methylpiperazin-1-yl)-3-pyridyl]-18,18-dimethyl-9,15-dioxo-4-triisopropylsilyloxy-16-oxa-10,22,28-triazapentacyclo[18.5.2.12,6.110,14.023,27]nonacosa-1(26),2,4,6(29),20,23(27),24-heptaen-8-yl]carbamate (compound G1, 500 mg) in THF (5 mL) was added TBAF (0.53 mL, 0.53 mmol, 1 M in THF) at 0° C. and stirred for 0.5 hour. After the reaction was complete, water (40 mL) was added, and the mixture was extracted with EtOAc (30 mL, three times). The combined organic phase was washed with brine (30 mL, four times), dried over anhydrous sodium sulfate, filtered and concentrated under vacuum to afford tert-butyl N-[(8S,14S)-22-ethyl-4-hydroxy-(21M)-21-[2-[(1S)-1-methoxyethyl]-5-(4-methylpiperazin-1-yl)-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamate (compound G2, 430 mg) as yellow gum. MS calc'd 838.5 (MH⁺), measured 838.6 (MH⁺)

### Step 2: Preparation of (8S,14S)-8-amino-22-ethyl-4-hydroxy-(21M)-21-[2-[(1S)-1-methoxyethyl]-5-(4-methylpiperazin-1-yl)-3-pyridyl]-18,18-dimethyl-16-oxa-10,22,28-triazapentacyclo[18.5.2.12,6.110,14.023,27]nonacosa-1(26),2,4,6(29),20,23(27),24-heptaene-9,15-dione (intermediate G)

To a solution of tert-butyl N-[(8S,14S)-22-ethyl-4-hydroxy-(21M)-21-[2-[(1S)-1-methoxyethyl]-5-(4-methylpiperazin-1-yl)-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamate (compound G2, 430 mg, 0.51 mmol) in DCM (4 mL) was added TFA (0.8 mL). The mixture was stirred at 20° C. for 12 h. The mixture was added into NaHCO₃ saturated aqueous solution (30 mL) and extracted with EtOAc (20 mL, three times). The combined organic phase was washed by brine (20 mL, twice), dried over anhydrous sodium sulfate, filtered and concentrated under vacuum to afford (8S,14S)-8-amino-22-ethyl-4-hydroxy-(21M)-21-[2-[(1S)-1-methoxyethyl]-5-(4-methylpiperazin-1-yl)-3-pyridyl]-18,18-dimethyl-16-oxa-10,22,28-triazapentacyclo[18.5.2.12,6.110,14.023,27]nonacosa-1(26),2,4,6(29),20,23(27),24-heptaene-9,15-dione (intermediate G, 360 mg) as yellow gum. MS calc'd 738.4 (MH⁺), measured 738.4 (MH⁺)

tert-butyl N-[(8S,14S)-22-ethyl-(21M)-21-[2-[(1S)-1-methoxyethyl]-5-(4-methylpiperazin-1-yl)-3-pyridyl]-18,18-dimethyl-9,15-dioxo-4-triisopropylsilyloxy-16-oxa-10,22,28-triazapentacyclo[18.5.2.12,6.110,14.023,27]nonacosa-1(26),2,4,6(29),20,23(27),24-heptaen-8-yl]carbamate (compound G1) was prepared in analogy to the preparation of Intermediate F by using methyl (3S)-1-[(2S)-2-(tert-butoxycarbonylamino)-3-[3-(4,4,5,5-tetramethyl-1,3,2-dioxaborolan-2-yl)-5-triisopropylsilyloxy-phenyl]propanoyl]hexahydropyridazine-3-carboxylate (Intermediate B) instead of methyl (3S)-1-[(2S)-3-(4-bromothiazol-2-yl)-2-(tert-butoxycarbonylamino)propanoyl]hexahydropyridazine-3-carboxylate (intermediate E).

### Intermediate H

### (7S,13S)-7-amino-21-ethyl-(20M)-20-[2-[(1S)-1-methoxyethyl]-3-pyridyl]-17,17-dimethyl-15-oxa-4-thia-9,21,27,28-tetrazapentacyclo[17.5.2.12,5.19,13.022,26]octacosa-1(25),2,5(28),19,22(26),23-hexaene-8,14-dione

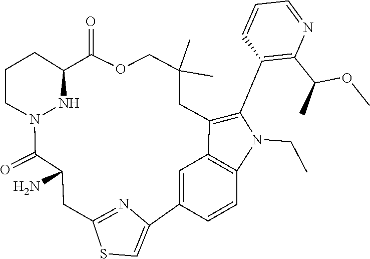

The title intermediate was prepared in analogy to the preparation of Intermediate F by using 3-[5-bromo-1-ethyl-(2M)-2-[2-[(1S)-1-methoxyethyl]-3-pyridyl]indol-3-yl]-2,2-dimethyl-propan-1-ol (intermediate A) instead of benzyl 4-[5-[5-bromo-1-ethyl-3-(3-hydroxy-2,2-dimethyl-propyl)indol-2-yl]-6-[(1S)-1-methoxyethyl]-3-pyridyl]piperazine-1-carboxylate (intermediate D).

### Intermediate I

### (7S,13S)-7-amino-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.12,5.19,13.022,26]octacosa-1(25),2,5(28),19,22(26),23-hexaene-8,14-dione

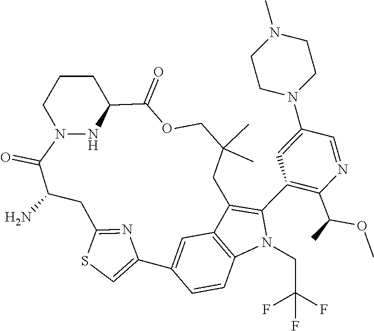

The title intermediate was prepared in analogy to the preparation of Intermediate F by using CF₃CH₂OTf instead of iodoethane.

### Example 1

### (3S)-1-[(2S)-2-chloro-2-fluoro-acetyl]-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6 (29),20,23 (27),24-heptaen-8-yl]carbamoyl]-2-methyl-propyl]-N-methyl-pyrrolidine-3-carboxamide

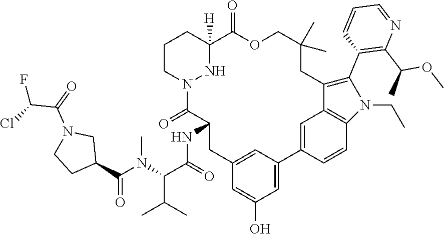

The title compound was prepared according to the following scheme:

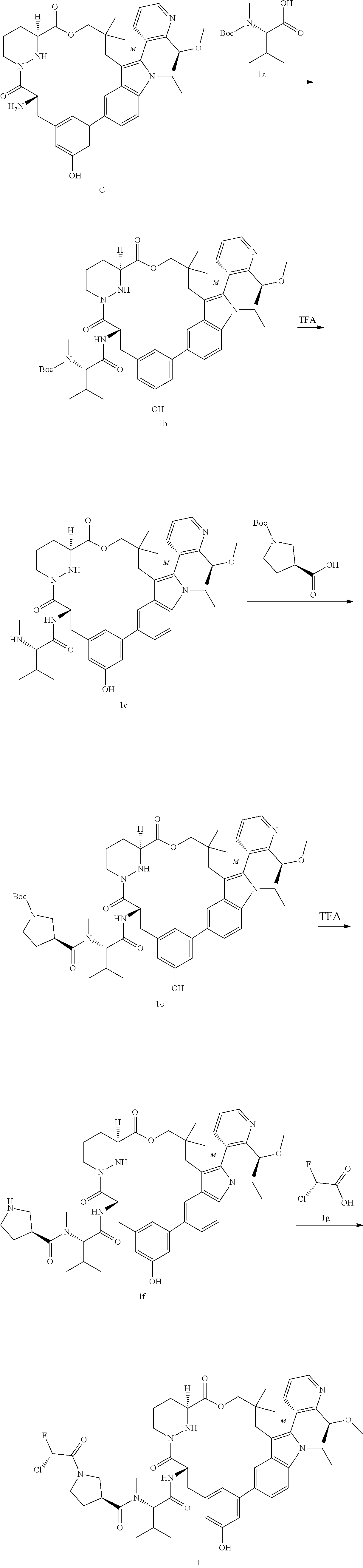

### Step 1: Preparation of tert-butyl N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-carbamate (compound 1b)

To a solution of BOC-N-ME-VAL-OH (compound 1a, 65.1 mg, 0.3 mmol), (8S,14S)-8-amino-22-ethyl-4-hydroxy-(21M)-21-[2-[(1S)-1-methoxyethyl]-3-pyridyl]-18,18-dimethyl-16-oxa-10,22,28-triazapentacyclo[18.5.2.12,6.110,14.023,27]nonacosa-1(26),2,4,6(29),20,23(27),24-heptaene-9,15-dione (intermediate C, 150.0 mg, 0.23 mmol) in DMF (1.5 mL) was added DIEA (90.9 mg, 0.7 mmol) and followed by HATU (55.1 mg, 0.23 mmol). After being stirred at 0° C. for 1 hr, the residue was diluted with water (40 mL) and extracted with EtOAc (60 mL) for 3 times. The combined organic phase was washed by brine (30 mL), dried over sodium sulfate, filtered and concentrated under vacuum. The residue was purified by silica gel column, eluted with ethyl acetate in petroleum ether=0˜20% to afford tert-butyl N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-carbamate (compound 1b, 180 mg) as yellow oil. MS: calc'd 853 (MH⁺), measured 853.2 (MH⁺).

### Step 2: Preparation of (2S)-N-[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]-3-methyl-2-(methylamino)butanamide (compound 1c)

To a solution of tert-butyl N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-carbamate (compound 1b, 180.0 mg, 0.19 mmol) in DCM (2 mL) was added TFA (0.4 mL). After being stirred at 20° C. for 1 hr, the mixture was poured into sat. aq. solution of NaHCO₃ (30 mL) and extracted with EtOAc (30 mL) for 3 times. The combined organic phase was washed by brine (30 mL), dried over sodium sulfate, filtered and concentrated under vacuum to afford (2S)-N-[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]-3-methyl-2-(methylamino)butanamide (compound 1c, 150 mg) as a yellow solid. MS: calc'd 775 (MNa⁺), measured 775.5 (MNa⁺).

### Step 3: Preparation of tert-butyl (3S)-3-[[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-methyl-carbamoyl]pyrrolidine-1-carboxylate (compound 1e)

To a solution of (S)-1-BOC-pyrrolidine-3-carboxylic acid (compound 1d, 54.11 mg, 0.250 mmol) and N,N-diisopropylethylamine (0.12 mL, 0.68 mmol) in DMF (2 mL) was added 0-(7-azabenzotriazol-1-yl)-N,N,N′,N′-tetramethyluronium hexafluorophosphate (88.23 mg, 0.23 mmol). The mixture was added to a solution of (2S)-N-[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]-3-methyl-2-(methylamino)butanamide (compound 1c, 145.6 mg, 0.19 mmol) in DMF (3 mL). The resulting mixture was stirred at 20° C. for 0.5 hrs. The mixture was then diluted with water (40 mL) and extracted with ethyl acetate (40 mL) for 3 times. The combined organic phase was washed by brine (40 mL), dried over anhydrous sodium sulfate, filtered and concentrated under vacuum. The residue was purified by silica gel column, eluted with ethyl acetate in petroleum ether=30%-100% to afford tert-butyl (3S)-3-[[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-methyl-carbamoyl]pyrrolidine-1-carboxylate (compound 1e, 140 mg). MS: calc'd 950 (MH⁺), measured 950.6 (MH⁺).

### Step 4: Preparation of (3S)-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-pyrrolidine-3-carboxamide (compound 1f)

To a solution of tert-butyl (3S)-3-[[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-methyl-carbamoyl]pyrrolidine-1-carboxylate(compound 1e, 140.0 mg, 0.13 mmol) in DCM (4 mL) was added TFA (0.8 mL). After being stirred at 20° C. for 0.5 hrs, the mixture was diluted with saturated aqueous solution of NaHCO₃ (30 mL) and extracted with ethyl acetate (40 mL) for 3 times. The combined organic phase was washed by brine (40 mL), dried over anhydrous sodium sulfate, filtered and the filtrate was concentrated under vacuum to afford (3S)-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-pyrrolidine-3-carboxamide (compound 1f, 110 mg) as a yellow solid. MS: calc'd 850 (MH⁺), measured 850.6 (MH⁺).

### Step 5: Preparation of (3S)-1-[(2S)-2-chloro-2-fluoro-acetyl]-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-pyrrolidine-3-carboxamide (example 1)

To a solution of (3S)-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-pyrrolidine-3-carboxamide (compound 1f, 150.0 mg, 0.2 mmol) and (2S)-2-chloro-2-fluoro-acetic acid (compound 1g, 59.5 mg, 0.5 mmol) in DMF (5 mL) was added N,N-diisopropylethylamine (137 mg, 1.1 mmol) and T₃P (168.4 mg, 0.26 mmol, 50% in EtOAc) at 0° C. After being stirred at 20° C. for 12 hrs, the mixture was diluted with water (20 mL) and extracted with ethyl acetate (20 mL) for 3 times. The combined organic phase was washed by brine (20 mL), dried over anhydrous sodium sulfate, filtered and the filtrate was concentrated under vacuum. The residue was purified by prep-HPLC to afford (3S)-1-[(2S)-2-chloro-2-fluoro-acetyl]-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-pyrrolidine-3-carboxamide (example 1, 51.1 mg) as an off-white solid. MS: calc'd 944 (MH⁺), measured 944.6 (MH⁺). ¹H NMR (400 MHz, CD₃OD) δ ppm 8.74-8.73 (m, 1H), 8.03 (s, 1H), 7.91-7.88 (m, 1H), 7.61 (d, J=8.4 Hz, 1H), 7.56-7.49 (m, 2H), 7.39 (s, 1H), 7.07-7.05 (m, 1H), 6.89-6.74 (m, 1H), 6.57-6.49 (m, 1H), 5.64-5.59 (m, 1H), 4.74 (d, J=11.2 Hz, 1H), 4.47 (d, J=13.6 Hz, 1H), 4.31-4.21 (m, 2H), 4.05-3.96 (m, 1H), 3.86-3.60 (m, 8H), 3.23-3.18 (m, 3H), 3.00-2.75 (m, 8H), 2.39-1.91 (m, 5H), 1.75-1.57 (m, 2H), 1.47 (d, J=6.4 Hz, 3H), 1.10-1.03 (m, 3H), 0.95-0.79 (m, 9H), 0.71-0.64 (m, 3H)

### Example 2

### (3S)-1-[(2R)-2-chloro-2-fluoro-acetyl]-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-pyrrolidine-3-carboxamide

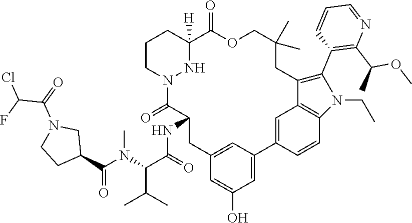

The title compound was prepared in analogy to the preparation of Example 1 by using (2R)-2-chloro-2-fluoro-acetic acid instead of (2S)-2-chloro-2-fluoro-acetic acid (compound 1g). Example 2 (21 mg) was obtained as a white solid. MS: calc'd 944 (MH⁺), measured 944.6 (MH⁺). ¹H NMR (400 MHz, CD3OD) δ 8.75-8.71 (m, 1H), 8.21-8.15 (m, 1H), 8.03 (d, J=5.2 Hz, 1H), 7.91-7.86 (m, 1H), 7.63-7.59 (m, 1H), 7.56-7.48 (m, 2H), 7.38 (s, 1H), 7.09-7.04 (m, 1H), 6.90-6.72 (m, 1H), 6.49 (d, J=9.2 Hz, 1H), 5.65-5.54 (m, 1H), 4.73 (s, 1H), 4.45 (d, J=12.4 Hz, 1H), 4.34-4.17 (m, 2H), 4.09-3.99 (m, 1H), 3.96-3.52 (m, 8H), 3.26-3.15 (m, 3H), 3.08-2.90 (m, 4.5H), 2.87-2.72 (m, 3.5H), 2.40-2.24 (m, 1H), 2.22-2.08 (m, 2H), 2.02-1.90 (m, 1H), 1.75-1.57 (m, 2H), 1.46 (d, J=6.0 Hz, 3H), 1.32-1.24 (m, 0.5H), 1.12-0.99 (m, 3.5H), 0.98-0.89 (m, 3H), 0.89-0.76 (m, 6H), 0.73-0.58 (m, 3H).

### Example 3

### N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-3-formyl-N-methyl-cyclobutanecarboxamide

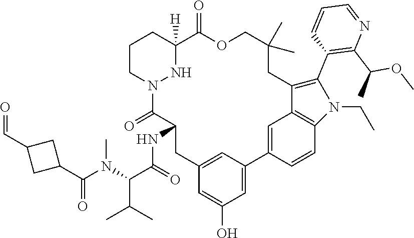

Compound 3e was prepared in analogy to the preparation of Example 1 by using 3-(methoxymethylene)cyclobutanecarboxylic acid (compound 3d) instead of (2S)-2-chloro-2-fluoro-acetic acid (compound 1g). Example 3 (8 mg) was converted from compound 3e and obtained as a yellow solid in the acidic condition of prep. HPLC. MS: calc'd 863 (MH⁺), measured 863.6 (MH⁺). ¹H NMR (400 MHz, CD₃OD) δ 8.78 (d, J=4.8 Hz, 1H), 8.45 (d, J=7.2 Hz, 1H), 8.04-7.92 (m 1H), 7.65-7.63 (m, 1H), 7.56-7.53 (m, 1H), 7.39 (s, 1H), 7.07 (s, 1H), 6.51 (s, 1H), 5.65-5.59 (m, 1H), 4.74-4.65 (m, 1H), 4.48-4.38 (m, 3H), 4.35-4.24 (m, 1H), 3.99-3.89 (m, 1H), 3.83-3.75 (m, 2H), 3.06-2.94 (m, 3H), 2.90-2.83 (m, 1H), 2.80-2.71 (m, 6H), 2.45-2.36 (m, 1H), 2.30-2.08 (m, 7H), 1.94-1.87 (m, 1H), 1.71-1.60 (m, 2H), 1.47 (d, J=6.0 Hz, 3H), 1.19 (s, 3H), 1.17-1.07 (m, 4H), 0.96-0.91 (m, 3H), 0.87-0.83 (m, 4H), 0.82-0.78 (m, 3H), 0.75-0.71 (m, 3H).

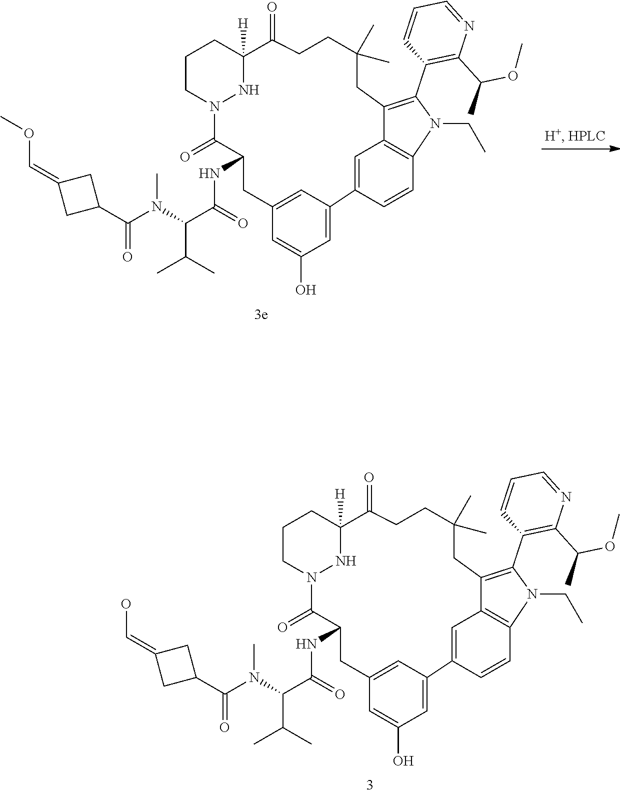

The compound 3d was prepared according to the following scheme:

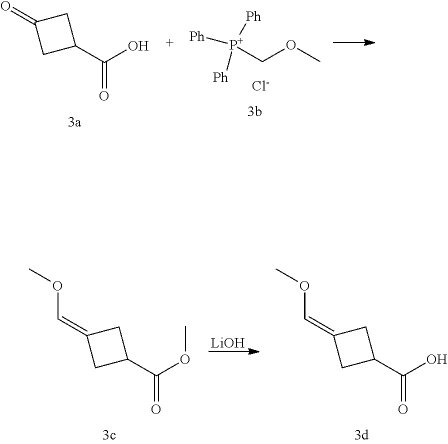

### Step 1: Preparation of methyl 3-(methoxymethylene)cyclobutanecarboxylate (compound 3c)

To a solution of (methoxymethyl)triphenylphosphonium chloride (compound 3b, 53.5 g, 156.09 mmol) in THF (500 mL) was added t-BuOK (17.5 g, 156.09 mmol) at 0° C. After 0.5 hr, methyl 3-oxocyclobutanecarboxylate (compound 3a, 10.0 g, 78.05 mmol) was added. After being stirred at 70° C. for 2.5 hrs, the mixture was acidified by 1 M aq. solution of HCl until PH=5-6 and extracted by EtOAc (300 mL) for 2 times. The organic phase was dried over anhydrous sodium sulfate, filtered and the filtrate was concentrated under vacuum. The residue was purified by silica gel column, eluted with ethyl acetate in petroleum ether=0˜10% to afford methyl 3-(methoxymethylene)cyclobutanecarboxylate (compound 3c, 2.5 g) as light yellow oil. ¹H NMR (400 MHz, DMSO-d₆) δ ppm 5.83-5.81 (m, 1H), 3.70 (s, 3H), 3.56 (s, 3H), 3.17-3.14 (m, 1H), 2.99-2.96 (m, 2H), 2.92-2.85 (m, 2H).

### Step 2: Preparation of 3-(methoxymethylene)cyclobutanecarboxylic acid (compound 3d)

To a solution of methyl 3-(methoxymethylene)cyclobutanecarboxylate (compound 3c, 2.0 g, 12.8 mmol) in THF (50 mL), methanol (10 mL) and water (10 mL) was added lithium hydroxide (3.1 mg, 128.1 mmol). After being stirred at 25° C. for 2 hrs, the mixture was diluted with water (30 mL) and extracted by DCM (100 mL) for 2 times. The organic phase was dried over anhydrous sodium sulfate, filtered and the filtrate was concentrated under reduced pressure to afford 3-(methoxymethylene)cyclobutanecarboxylic acid (compound 3d, 1.0 g) as light yellow oil. ¹H NMR (400 MHz, DMSO-d₆) δ ppm 5.89-5.87 (m, 1H), 3.47 (s, 3H), 3.10-3.01 (m, 1H), 2.88-2.79 (m, 1H), 2.78-2.70 (m, 3H).

### Example 4

### N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-3-ethynyl-N-methyl-cyclobutanecarboxamide

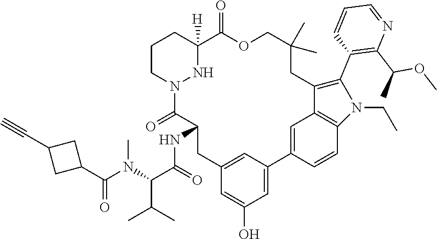

The title compound was prepared in analogy to the preparation of Example 1 by using 3-ethynylcyclobutanecarboxylic acid instead of (S)-1-BOC-pyrrolidine-3-carboxylic acid (compound 1d). Example 4 (20 mg) was obtained as a brown solid. MS: calc'd 859 (MH⁺), measured 859.5 (MH⁺). ¹H NMR (400 MHz, CD₃OD) δ ppm 8.73 (dd, J=1.6, 4.8 Hz, 1H), 8.15-8.10 (m, 1H), 8.01-7.99 (m 1H), 7.90-7.87 (m, 1H), 7.61-7.48 (m, 3H), 7.37-7.35 (m, 1H), 7.06-7.04 (m, 1H), 6.51-6.48 (m, 1H), 5.65-5.57 (m, 1H), 4.71-4.66 (m, 1H), 4.46-4.43 (m, 1H), 4.33-4.21 (m, 2H), 4.07-4.01 (m, 1H), 3.81-3.62 (m, 3H), 3.49-3.36 (m, 1H), 3.26-3.21 (m, 3H), 3.07-2.87 (m, 3H), 2.84-2.66 (m, 6H), 2.65-2.38 (m, 4H), 2.37-2.25 (m, 1H), 2.23-2.03 (m, 3H), 1.94-1.88 (m, 1H), 1.71-1.56 (m, 2H), 1.47 (d, J=6.0 Hz, 3H), 1.36-1.19 (m, 1H), 1.06-1.01 (m, 3H), 0.98-0.86 (m, 3H), 0.85-0.76 (m, 6H), 0.65-0.58 (m, 3H).

### Example 5

### (3S)-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-1-(2-oxopropanoyl)pyrrolidine-3-carboxamide

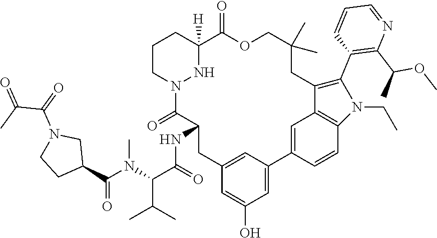

The title compound was prepared in analogy to the preparation of Example 1 by using pyruvic acid instead of (2S)-2-chloro-2-fluoro-acetic acid (compound 1g). Example 5 (8 mg) was obtained as a white solid. MS: calc'd 920 (MH⁺), measured 920.5 (MH⁺). ¹H NMR (400 MHz, CD₃OD) δ ppm 8.73 (dd, J=1.2, 4.8 Hz, 1H), 8.03 (s, 1H), 7.90-7.88 (m 1H), 7.62-7.60 (m, 1H), 7.56-7.49 (m, 2H), 7.39-7.37 (m, 1H), 7.07-7.05 (m, 1H), 6.65-6.45 (m, 1H), 5.69-5.55 (m, 1H), 4.75-4.70 (m, 1H), 4.53-4.44 (m, 1H), 4.35-4.15 (m, 2H), 4.13-3.98 (m, 1H), 3.93-3.82 (m, 1H), 3.81-3.72 (m, 3H), 3.68-3.55 (m, 3H), 3.26-3.21 (m, 3H), 3.05-2.95 (m, 1H), 2.94-2.88 (m, 3H), 2.85-2.72 (m, 3H), 2.42-2.40 (m, 2H), 2.36-2.21 (m, 1H), 2.18-1.90 (m, 4H), 1.76-1.55 (m, 2H), 1.47 (d, J=6.0 Hz, 3H), 1.36-1.19 (m, 2H), 1.10-0.96 (m, 4H), 0.95-0.93 (m, 2H), 0.91-0.86 (m, 1H), 0.84-0.79 (m, 5H), 0.76-0.71 (m, 1H), 0.65-0.58 (m, 3H).

### Example 6

### (3S)-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-1-prop-2-ynyl-pyrrolidine-3-carboxamide

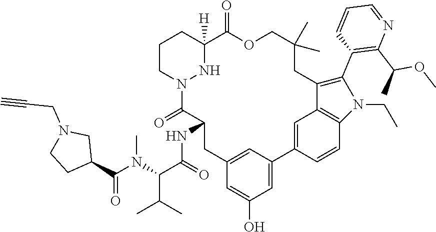

The title compound was prepared in analogy to the preparation of Example 1 by using (2S)-3-methyl-2-[methyl-[(3S)-1-prop-2-ynylpyrrolidine-3-carbonyl]amino]butanoic acid (compound 6h) instead of BOC-N-ME-VAL-OH (compound 1a). Example 6 (27 mg) was obtained as a white solid. MS: calc'd 888 (MH⁺), measured 888.7 (MH⁺). ¹H NMR (400 MHz, CD₃OD) δ ppm 8.80-8.78 (m, 1H), 8.09-7.92 (m, 2H), 7.69-7.33 (m, 4H), 7.09-7.05 (m, 1H), 6.60-6.48 (m, 1H), 5.63-5.36 (m, 1H), 4.73-4.65 (m, 1H), 4.44-4.21 (m, 4H), 4.10-3.99 (m, 2H), 3.84-3.38 (m, 8H), 3.27-2.97 (m, 8H), 2.85-2.73 (m, 6H), 2.20-2.11 (m, 2H), 1.97-1.90 (m, 1H), 1.70-1.58 (m, 2H), 1.48 (dd, J=2.0, 6.0 Hz, 3H), 1.36-1.29 (m, 1H), 1.19 (t, J=7.2 Hz, 1H), 1.12-0.83 (m, 10H), 0.70-0.63 (m, 3H).

The compound 6h was prepared according to the following scheme:

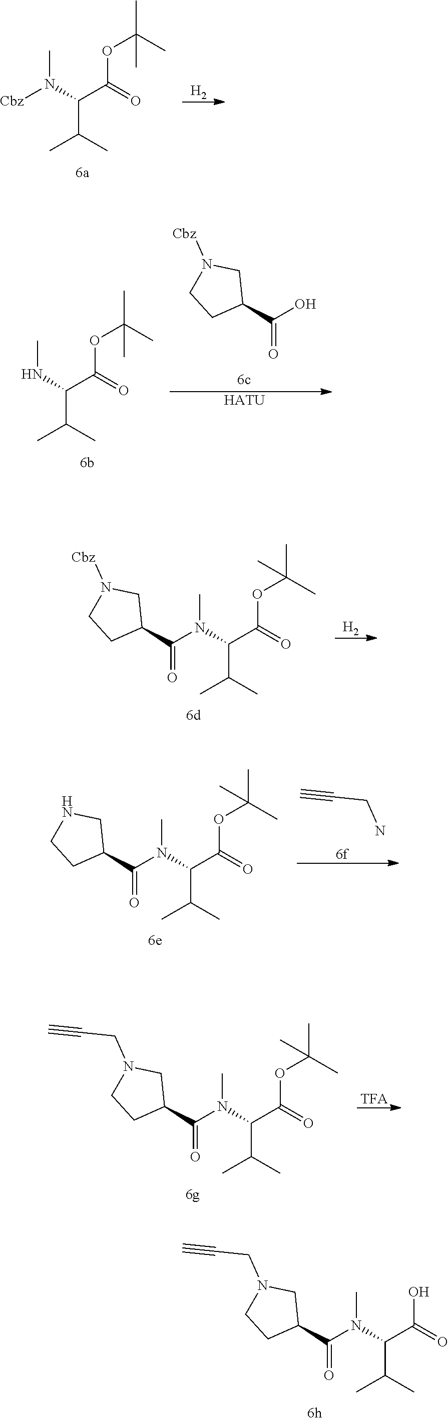

### Step 1: Preparation of tert-butyl (2S)-3-methyl-2-(methylamino)butanoate (compound 6b)

To a solution of tert-butyl (2S)-2-[benzyloxycarbonyl(methyl)amino]-3-methyl-butanoate (compound 6a, 10.0 g, 31.11 mmol) in ethyl acetate (150 mL) was added Pd/C (1.0 g, 10% purity). The mixture was degassed under vacuum and purged H₂ for 3 times. The resulting mixture was stirred for 12 hours at 20° C. under H₂ balloon. TLC (PE:EtOAc=3:1) showed starting material was consumed completely. The mixture was filtered and the filtrate was concentrated under vacuum to afford tert-butyl (2S)-3-methyl-2-(methylamino)butanoate (compound 6b, 5.2 g) as colorless oil which can be used in the next step without purification.

### Step 2: Preparation of benzyl (3S)-3-[[(1S)-1-tert-butoxycarbonyl-2-methyl-propyl]-methyl-carbamoyl]pyrrolidine-1-carboxylate (compound 6d)

To a solution of tert-butyl (2S)-3-methyl-2-(methylamino)butanoate (compound 6b, 4.1 g, 22.07 mmol) and (3S)-1-benzyloxycarbonylpyrrolidine-3-carboxylic acid (compound 6c, 5.0 g, 20 mmol) in ethyl acetate (50 mL) was added DIEA (10.4 mL, 60.1 mmol) and T₃P (19.1 g, 30.1 mmol, 50% in EtOAc) at 0° C. After being stirred for 1 hour at 0° C., the reaction mixture was poured into water (300 mL) and extracted with EtOAc (200 mL) for 2 times. The combined organic layer was washed with brine (200 mL), dried over anhydrous sodium sulfate, filtered and the filtrate was concentrated under reduced pressure. The residue was purified by silica gel column, eluted with EtOAc in petroleum ether=30˜50% to afford benzyl (3S)-3-[[(1S)-1-tert-butoxycarbonyl-2-methyl-propyl]-methyl-carbamoyl]pyrrolidine-1-carboxylate (compound 6d, 7 g) as colorless oil. MS: calc'd 419 (MH⁺1), measured 419.2 (MH⁺).

### Step 3: Preparation of tert-butyl (2S)-3-methyl-2-[methyl-[(3S)-pyrrolidine-3-carbonyl]amino]butanoate (compound 6e)

To a solution of benzyl (3S)-3-[[(1S)-1-tert-butoxycarbonyl-2-methyl-propyl]-methyl-carbamoyl]pyrrolidine-1-carboxylate (compound 6d, 4.0 g, 9.56 mmol) in ethyl acetate (50 mL) was added Pd/C (0.4 g, 10% purity). The mixture was degassed under vacuum and purged H₂ for 3 times. After being stirred for 12 hours at 20° C. under H₂ balloon, the reaction mixture was filtered and the filtrate was concentrated under vacuum to afford tert-butyl (2S)-3-methyl-2-[methyl-[(3S)-pyrrolidine-3-carbonyl]amino]butanoate (compound 6e, 2.3 g) as colorless oil. MS: calc'd 285 (MH⁺), measured 285.1 (MH⁺).

### Step 4: Preparation of tert-butyl (2S)-3-methyl-2-[methyl-(1-prop-2-ynylpyrrolidine-3-carbonyl)amino]butanoate (compound 6g)

To a solution of tert-butyl (2S)-3-methyl-2-[methyl(pyrrolidine-3-carbonyl)amino]butanoate (compound 6e, 100.0 mg, 0.350 mmol) and potassium carbonate (48.6 mg, 0.35 mmol) in MeCN (2.5 mL) was added propargyl bromide (compound 6e, 52.2 mg, 0.35 mmol). After being stirred for 25° C. for 1 hour, the reaction mixture was diluted with water (50 mL) and extracted with ethyl acetate (50 mL) for 2 times. The combined organic layer was washed with brine (50 mL), dried over anhydrous sodium sulfate, filtered and the filtrate was concentrated under vacuum to give a residue. The residue was purified by reversed-phase flash, eluted with ACN in water (0.1% TFA)=15˜35% to afford tert-butyl (2S)-3-methyl-2-[methyl-(1-prop-2-ynylpyrrolidine-3-carbonyl)amino]butanoate (compound 6g, 70 mg) was obtained as colorless oil. MS: calc'd 323 (MH⁺), measured 322.9 (MH⁺).

### Step 5: Preparation of (2S)-3-methyl-2-[methyl-[(3S)-1-prop-2-ynylpyrrolidine-3-carbonyl]amino]butanoic acid (compound 6h)

tert-butyl (2S)-3-methyl-2-[methyl-[(3S)-1-prop-2-ynylpyrrolidine-3-carbonyl]amino]butanoate (compound 6g, 70 mg, 0.22 mmol) in TFA (0.5 mL) was stirred at 25° C. for 1.5 hrs. The mixture was concentrated under vacuum to give (2S)-3-methyl-2-[methyl-[(3S)-1-prop-2-ynylpyrrolidine-3-carbonyl]amino]butanoic acid (compound 6h, 75 mg) as light yellow oil. MS: calc'd 267 (MH⁺), measured 267.1 (MH⁺).

### Example 7

### (3S)-1-(cyanomethyl)-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-pyrrolidine-3-carboxamide

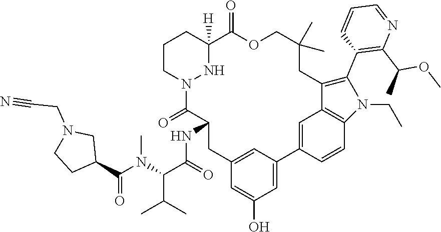

The title compound was prepared in analogy to the preparation of Example 6 by using 2-bromoacetonitrile instead of 3-bromoprop-1-yne (compound 6f). Example 7 (42 mg) was obtained as a white solid. MS: calc'd 889 (MH⁺), measured 889.6 (MH⁺). ¹H NMR (400 MHz, CD₃OD) δ ppm 8.85-8.82 (m, 1H), 8.21-8.17 (m, 1H), 8.07-8.03 (m, 1H), 7.79-7.75 (m, 1H), 7.66-7.62 (m, 1H), 7.56-7.54 (m, 1H), 7.10-7.06 (m, 1H), 7.44-7.41 (m, 1H), 6.67-6.64 (0.6H), 6.50 (s, 0.4H), 5.65-5.59 (m, 1H), 4.73-4.67 (m, 1H), 4.50-4.25 (m, 6H), 4.04-3.97 (m, 1H), 3.84-3.66 (m, 5H), 3.50-3.40 (m, 2H), 3.14-2.73 (m, 11H), 2.60-2.48 (m, 1H), 2.25-2.09 (m, 3H), 1.98-1.90 (m, 1H), 1.78-1.59 (m, 2H), 1.48 (d, J=6.4 Hz, 3H), 1.33-1.29 (m, 1H), 1.13-1.06 (m, 3H), 0.95-0.93 (m, 1H), 0.92-0.85 (m, 7H), 0.71 (d, J=6.8 Hz, 3H).

### Example 8

### 3-[2-[dimethyl(oxo)-λ⁶-sulfanylidene]acetyl]-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-cyclobutanecarboxamide

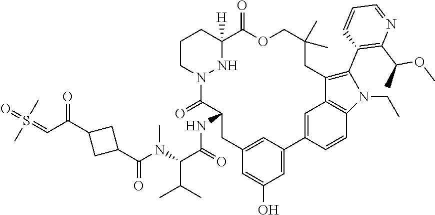

The title compound was prepared in analogy to the preparation of Example 1 by using (2S)-2-[[3-[2-[dimethyl(oxo)-λ⁶-sulfanylidene]acetyl]cyclobutanecarbonyl]-methyl-amino]-3-methyl-butanoic acid (compound 8f) instead of BOC-N-ME-VAL-OH (compound 1a). Example 8 (8 mg) was obtained as a yellow solid. MS: calc'd 953 (MH⁺), measured 953.6 (MH⁺). ¹H NMR (400 MHz, CD₃OD) δ ppm 8.77 (d, J=4.8 Hz, 1H), 8.03-7.97 (m, 2H), 7.64-7.58 (m, 2H), 7.53 (d, J=10.4 Hz, 1H), 7.40-7.37 (m, 1H), 7.06-7.03 (m, 1H), 6.64-6.50 (m, 1H), 5.67-5.59 (m, 1H), 4.73-4.75 (m, 1H), 4.48-4.41 (m, 1H), 4.34-4.22 (m, 2H), 4.10-4.00 (m, 1H), 3.94-3.87 (m, 4H), 3.79-3.75 (m, 1H), 3.65-3.45 (m, 5H), 3.25-3.21 (m, 3H), 3.08-2.70 (m, 9H), 2.65-2.51 (m, 3H), 2.20-2.05 (m, 2H), 1.95-1.88 (m, 1H), 1.71-1.57 (m, 2H), 1.48 (d, J=6.0 Hz, 3H), 1.34-1.27 (m, 1H), 1.19 (t, J=7.2 Hz, 1H), 1.10-1.04 (m, 3H), 0.95-0.94 (m, 1H), 0.88-0.82 (m, 8H), 0.72-0.64 (m, 3H).

The compound 8f was prepared according to the following scheme:

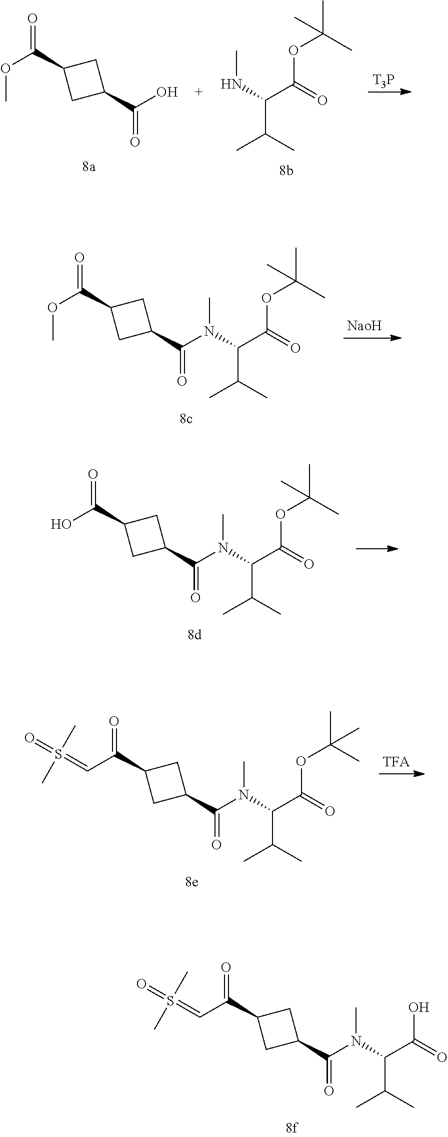

### Step 1: Preparation of methyl 3-[[(1S)-1-tert-butoxycarbonyl-2-methyl-propyl]-methyl-carbamoyl]cyclobutanecarboxylate (compound 8c)

To a solution of tert-butyl (2S)-3-methyl-2-(methylamino)butanoate (compound 8b, 1 g, 5.3 mmol) and 3-methoxycarbonylcyclobutanecarboxylic acid (compound 8a, 844.4 mg, 5.3 mmol) in ethyl acetate (15 mL) was added DIEA (2.7 mL, 16.02 mmol) and T₃P (5.1 g, 8.01 mmol, 50% in EtOAc) at 0° C. After being stirred for 1 hour at 0° C., the mixture was poured into water (50 mL) and extracted with EtOAc (50 mL) for 2 times. The combined organic layer was washed with brine (50 mL) and dried over anhydrous sodium sulfate, filtered and the filtrate was concentrated under vacuum. The residue was purified by silica gel column, eluted with petroleum ether:EtOAc=15˜35% to afford methyl 3-[[(1S)-1-tert-butoxycarbonyl-2-methyl-propyl]-methyl-carbamoyl]cyclobutanecarboxylate (compound 8c, 740 mg) as colorless oil. ¹H NMR (400 MHz, CD₃OD): δ ppm 4.57 (d, J=10.4 Hz, 0.6 Hz), 3.65 (s, 3H), 3.52-3.43 (m, 1H), 2.96-3.05 (m, 1H), 2.83-2.72 (m, 3H), 2.44-2.33 (m, 4H), 2.19-2.10 (m, 1H), 1.42 (s, 9H), 0.97 (d, J=6.4 Hz, 3H), 0.80 (d, J=6.4 Hz, 3H).

### Step 2: Preparation of 3-[[(1S)-1-tert-butoxycarbonyl-2-methyl-propyl]-methyl-carbamoyl]cyclobutanecarboxylic acid (compound 8d)

To a solution of methyl 3-[[(1S)-1-tert-butoxycarbonyl-2-methyl-propyl]-methyl-carbamoyl]cyclobutanecarboxylate (compound 8c, 700 mg, 2.14 mmol) in THF (6 mL) and water (1.5 mL) was added NaOH (171 mg, 4.2 mmol) at 0° C. After being stirred for 2 hours at 20° C., the mixture was poured into 1 M aq. solution of HCl solution (50 mL) and extracted with EtOAc (50 mL) for 2 times. The combined organic phase was washed with brine (50 mL) and dried over anhydrous sodium sulfate, filtered and the filtrate was concentrated under vacuum to afford 3-[[(1S)-1-tert-butoxycarbonyl-2-methyl-propyl]-methyl-carbamoyl]cyclobutanecarboxylic acid (compound 8d, 670 mg) as yellow oil. MS: calc'd 314 (MH⁺), measured 314.1 (MH⁺).

### Step 3: Preparation of tert-butyl (2S)-2-[[3-[2-[dimethyl(oxo)-λ⁶-sulfanylidene]acetyl]cyclobutanecarbonyl]-methyl-amino]-3-methyl-butanoate (compound 8e)

To a solution of trimethylsulfoxonium iodide (421.3 mg, 1.9 mmol) in THF (3 mL) was added t-BuOK (214.4 mg, 1.9 mmol) to form a mixture A which was stirred at 65° C. for 2 hours under N₂ protection, then cooled to 0° C. In another flask, 3-[[(1S)-1-tert-butoxycarbonyl-2-methyl-propyl]-methyl-carbamoyl]cyclobutanecarboxylic acid (compound 8d, 200 mg, 0.64 mmol) in THF (3 mL) was added HATU (266.7 mg, 0.7 mmol) and Et₃N (96.6 mg, 0.96 mmol) to form a mixture B, which was stirred at 25° C. for 2 hours. Then the mixture B was added into the mixture A at 0° C. The resulting reaction mixture was stirred at 5° C. for another 4 hours, then concentrated under vacuum. The residue was purified with prep-HPLC to afford tert-butyl (2S)-2-[[3-[2-[dimethyl(oxo)-λ⁶-sulfanylidene]acetyl]cyclobutanecarbonyl]-methyl-amino]-3-methyl-butanoate (compound 8e, 60 mg) as colorless oil. MS: calc'd 388 (MH⁺), measured 388.0 (MH⁺).

### Step 4: Preparation of (2S)-2-[[3-[2-[dimethyl(oxo)-λ⁶-sulfanylidene]acetyl]cyclobutanecarbonyl]-methyl-amino]-3-methyl-butanoic acid (compound 8f)

To a solution of tert-butyl (2S)-2-[[3-[2-[dimethyl(oxo)-λ⁶-sulfanylidene]acetyl]cyclobutanecarbonyl]-methyl-amino]-3-methyl-butanoate (compound 8e, 20 mg, 0.05 mmol) in DCM (3 mL) was added TFA (1 mL). The mixture was stirred at 20° C. for 2 hours. The mixture was concentrated under vacuum to afford (2S)-2-[[3-[2-[dimethyl(oxo)-λ⁶-sulfanylidene]acetyl]cyclobutanecarbonyl]-methyl-amino]-3-methyl-butanoic acid (compound 8f, 17 mg) as yellow oil. MS obsd. MS: calc'd 331.8 (MH⁺), measured 331.9 (MH⁺).

### Example 9

### (3S)-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-1-[(E)-3-(1,2,4-triazol-1-yl)prop-2-enoyl]pyrrolidine-3-carboxamide

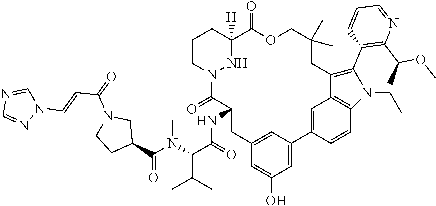

The compound was prepared according to the following scheme

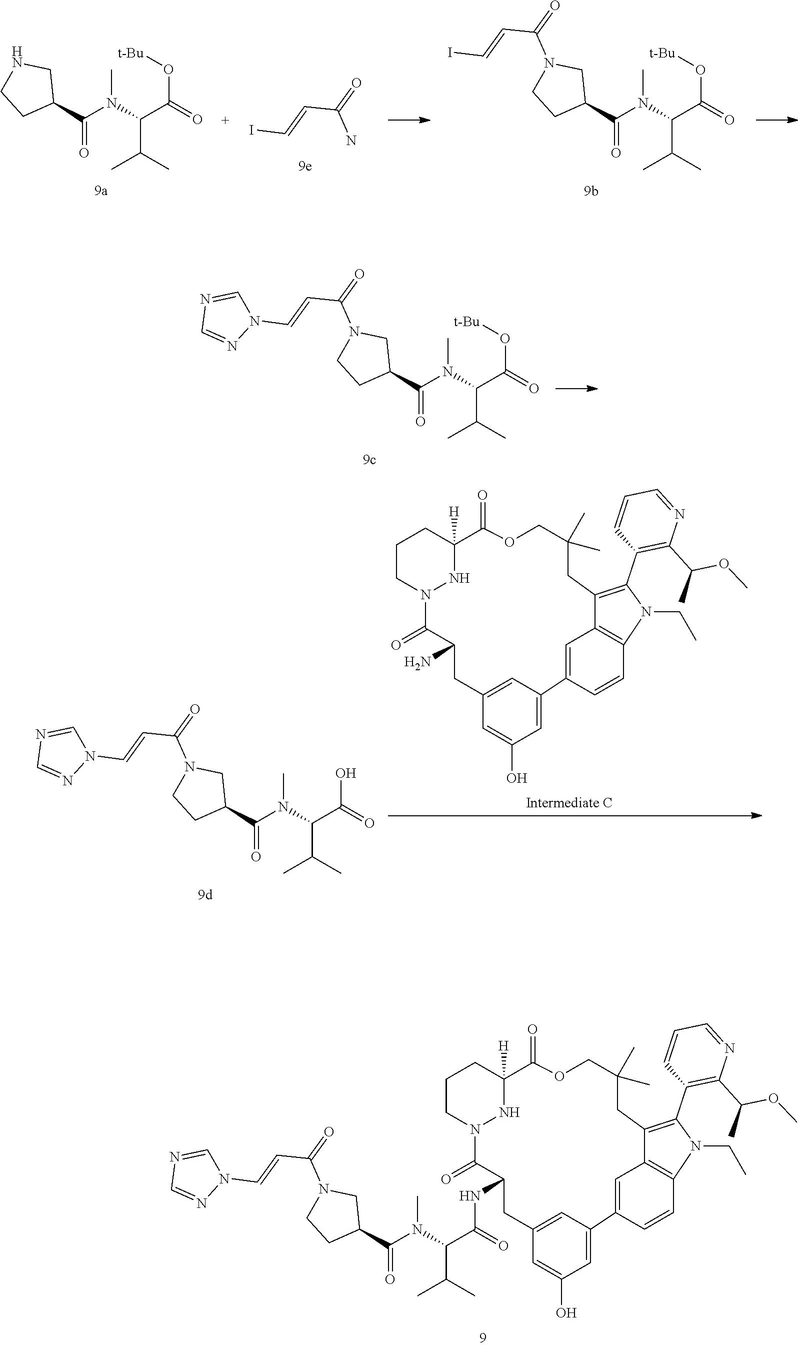

### Step 1: Preparation of tert-butyl (2S)-2-[[(3S)-1-[(E)-3-iodoprop-2-enoyl]pyrrolidine-3-carbonyl]-methyl-amino]-3-methyl-butanoate (compound 9b)

To a solution of (E)-3-iodoprop-2-enoic acid (compound 9e, 250.0 mg, 1.26 mmol) in DMF (4 mL) was added tert-butyl (2S)-3-methyl-2-[methyl-[(3S)-pyrrolidine-3-carbonyl]amino]butanoate (compound 9a, 359 mg, 1.26 mmol), DIEA (488.74 mg, 3.79 mmol) and T₃P (1044 mg, 1.64 mmol). After being stirred at 25° C. for 3 hours, the mixture was diluted with EtOAc (50 mL) and poured into water (30 mL). After separation, the aqueous phase was extracted with EA (30 mL, three times). The combined organic phase was dried over Na₂SO₄. After filtration and concentration, the residue was purified with silica gel column eluted with PE:EA=1:10 to afford tert-butyl (2S)-2-[[(3S)-1-[(E)-3-iodoprop-2-enoyl]pyrrolidine-3-carbonyl]-methyl-amino]-3-methyl-butanoate (compound 9b, 350 mg) as colorless oil. MS calc'd 465.3 (MH⁺), measured 465.2 (MH⁺).

### Step 2: Preparation of tert-butyl (2S)-3-methyl-2-[methyl-[(3S)-1-[(E)-3-(1,2,4-triazol-1-yl)prop-2-enoyl]pyrrolidine-3-carbonyl]amino]butanoate (compound 9c)

To a solution of tert-butyl (2S)-2-[[(3S)-1-[(E)-3-iodoprop-2-enoyl]pyrrolidine-3-carbonyl]-methyl-amino]-3-methyl-butanoate (compound 9b, 150 mg, 0.32 mmol) in DMF (3 mL) was added DABCO (72 mg, 0.65 mmol). After being stirred at 25° C. for 10 minutes, 1,2,4-triazole (33 mg, 0.48 mmol) was added into the mixture and the resulting mixture was stirred at 25° C. for 16 hours. The mixture was diluted with EtOAc (50 mL) and poured into water (30 mL). The aqueous phase was extracted with EtOAc (30 mL, three times). The combined organic phase was washed with EtOAc (30 mL, three times), dried over Na₂SO₄, filtered and concentrated, the residue was purified with silica gel column (MeOH in DCM=0˜5%) to afford tert-butyl (2S)-3-methyl-2-[methyl-[(3S)-1-[(E)-3-(1,2,4-triazol-1-yl)prop-2-enoyl]pyrrolidine-3-carbonyl]amino]butanoate (compound 9c, 40 mg) as yellow oil. MS calc'd 406.2 (MH⁺), measured 406.1 (MH⁺).

### Step 3: Preparation of (2S)-3-methyl-2-[methyl-[(3S)-1-[(E)-3-(1,2,4-triazol-1-yl)prop-2-enoyl]pyrrolidine-3-carbonyl]amino]butanoic acid (compound 9d)

To a solution of tert-butyl (2S)-3-methyl-2-[methyl-[(3S)-1-[(E)-3-(1,2,4-triazol-1-yl)prop-2-enoyl]pyrrolidine-3-carbonyl]amino]butanoate (compound 9c, 30 mg, 0.07 mmol) in DCM (4 mL) was added TFA (1.0 mL). After being stirred at 20° C. for 1 hour, the mixture was concentrated under vacuum to afford (2S)-3-methyl-2-[methyl-[(3S)-1-[(E)-3-(1,2,4-triazol-1-yl)prop-2-enoyl]pyrrolidine-3-carbonyl]amino]butanoic acid (compound 9d, 34 mg) as yellow oil. MS calc'd 350.2 (MH⁺), measured 350.0 (MH⁺).

### Step 4: (3S)-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-1-[(E)-3-(1,2,4-triazol-1-yl)prop-2-enoyl]pyrrolidine-3-carboxamide (Example 9)

To a solution of (2S)-3-methyl-2-[methyl-[(3S)-1-[(E)-3-(1,2,4-triazol-1-yl)prop-2-enoyl]pyrrolidine-3-carbonyl]amino]butanoic acid (compound 9d, 10 mg, 0.03 mmol) in DMF (2 mL) was added DIEA (16 mg, 0.13 mmol) and HATU (15 mg, 0.04 mmol). After being stirred for 10 min, the mixture was added (3S)-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-1-[(E)-3-(1,2,4-triazol-1-yl)prop-2-enoyl]pyrrolidine-3-carboxamide (20 mg, 0.03 mmol) and stirred for another 2 hours. After the reaction was complete, the mixture was purified by prep-HPLC to afford (3S)-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-1-[(E)-3-(1,2,4-triazol-1-yl)prop-2-enoyl]pyrrolidine-3-carboxamide (Example 9, 8 mg) as a white solid. MS calc'd 971.5 (MH⁺), measured 971.7 (MH⁺). ¹H NMR (400 MHz, METHANOL-d₄) δ ppm 9.02-9.09 (m, 1H), 8.86-8.90 (m, 1H), 8.33-8.43 (m, 1H), 8.08-8.12 (m, 1H), 7.85-7.94 (m, 1H), 7.62-7.69 (m, 1H), 7.54-7.58 (m, 1H), 7.39-7.44 (m, 1H), 7.26-7.32 (m, 1H), 7.03-7.07 (m, 1H), 6.6-6.65 (m, 1H), 6.07-6.15 (m, 1H), 5.59-5.65 (m, 1H), 4.29-4.48 (m, 1H), 3.94-4.02 (m, 1H), 3.56-3.85 (m, 8H), 2.90-3.06 (m, 6H), 2.69-2.85 (m, 4H), 2.05-2.32 (m, 4H), 1.88-1.96 (m, 1H), 1.57-1.71 (m, 2H), 1.43-1.52 (m, 4H), 1.04-1.16 (m, 4H), 0.73-0.98 (m, 15H)

### Example 10

### (3S)-1-[(2R)-2-chloro-2-fluoro-acetyl]-N-[(1S)-1-[[(7S,13S)-21-ethyl-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.12,5.19,13.022,26]octacosa-1(25),2,5(28),19,22(26),23-hexaen-7-yl]carbamoyl]-2-methyl-propyl]-N-methyl-pyrrolidine-3-carboxamide

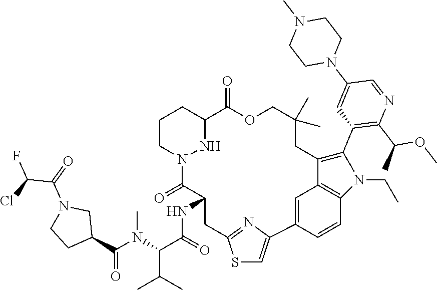

The title compound was prepared in analogy to the preparation of Example 1 by using (2R)-2-chloro-2-fluoro-acetic acid and (7S,13S)-7-amino-21-ethyl-(20M)-20-[2-[(1S)-1-methoxyethyl]-5-(4-methylpiperazin-1-yl)-3-pyridyl]-17,17-dimethyl-15-oxa-4-thia-9,21,27,28-tetrazapentacyclo[17.5.2.12,5.19,13.022,26]octacosa-1(25),2,5(28),19,22(26),23-hexaene-8,14-dione (intermediate F) instead of (2S)-2-chloro-2-fluoro-acetic acid (compound 1g) and (8S,14S)-8-amino-22-ethyl-4-hydroxy-(21M)-21-[2-[(1S)-1-methoxyethyl]-3-pyridyl]-18,18-dimethyl-16-oxa-10,22,28-triazapentacyclo[18.5.2.12,6.110,14.023,27]nonacosa-1(26),2,4,6(29),20,23(27),24-heptaene-9,15-dione (intermediate C). Example 10 (89 mg) as a yellow solid. MS calc'd 1033.5 (MH⁺), measured 1033.7 (MH⁺). ¹H NMR (400 MHz, MeOD) δ ppm 8.61-8.54 (m, 1H), 8.49 (d, J=2.4 Hz, 1H), 7.76-7.70 (m, 2H), 7.59-7.49 (m, 2H), 6.93-6.74 (m, 1H), 5.85-5.67 (m, 1H), 4.84-4.76 (m, 1H), 4.47-4.31 (m, 3H), 4.27-4.19 (m, 1H), 4.18-4.04 (m, 2H), 3.98-3.86 (m, 1H), 3.85-3.65 (m, 7H), 3.64-3.45 (m, 4H), 3.42-3.35 (m, 5H), 3.28-3.23 (m, 1H), 3.18-3.11 (m, 1H), 3.10 (d, J=4.0 Hz, 3H), 2.99 (s, 3H), 2.80 (dt, J=12.8, 2.8 Hz, 1H), 2.69-2.56 (m, 1H), 2.39-2.22 (m, 3H), 2.20-2.02 (m, 1H), 2.01-1.90 (m, 1H), 1.88-1.72 (m, 1H), 1.69-1.55 (m, 1H), 1.45 (d, J=6.0 Hz, 3H), 1.11-1.01 (m, 1H), 1.01-0.93 (m, 9H), 0.87 (d, J=6.4 Hz, 3H), 0.56-0.46 (m, 3H).

### Example 11

### (3S)-1-(bicyclo[1.1.0]butane-1-carbonyl)-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-pyrrolidine-3-carboxamide

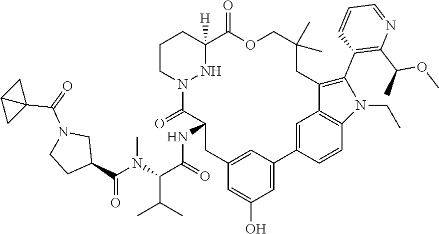

The compound was prepared according to the following scheme:

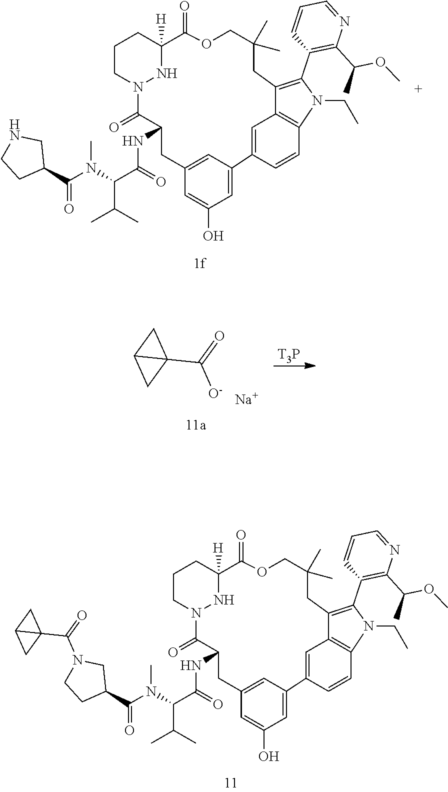

To a solution of bicyclo[1.1.0]butane-1-carbonyloxy sodium (compound 11a, 35 mg, 0.29 mmol), (3S)-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-pyrrolidine-3-carboxamide (compound 1f, 50 mg, 0.06 mmol) in DMF (1.5 mL) was added DIEA (38 mg, 0.29 mmol), T₃P (28 mg, 0.09 mmol) at 0° C. The mixture was stirred at 20° C. for 12 hours. After the reaction was complete, water (40 mL) was added and the mixture was extracted with EtOAc (30 mL, three times). The combined organic phase was washed with brine (30 mL, four times), dried over anhydrous sodium sulfate, filtered and concentrated under vacuum to give a residue, which was purified by prep-HPLC to afford (3S)-1-(bicyclo[1.1.0]butane-1-carbonyl)-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-pyrrolidine-3-carboxamide (EXAMPLE 11, 4 mg) as a white solid. MS calc'd 930.5 (MH⁺), measured 930.5 (MH⁺). ¹H NMR (400 MHz, Methanol-d4) δ ppm 8.75-8.72 (m, 1H), 8.32 (s, 1H), 8.03 (s, 1H), 7.93-7.86 (m, 1H), 7.62-7.58 (m, 1H), 7.56-7.53 (m, 1H), 7.52-7.49 (m, 1H), 7.41-7.33 (m, 1H), 7.10-7.04 (m, 1H), 6.49 (s, 1H), 5.66-5.55 (m, 1H), 5.45-5.22 (m, 1H), 4.78-4.72 (m, 2H), 4.46 (d, J=13.2 Hz, 1H), 4.34-4.18 (m, 2H), 3.84-3.68 (m, 4H), 3.25-3.16 (m, 3H), 3.00-2.90 (m, 4H), 2.86-2.89 (m, 4H), 2.33-2.23 (m, 3H), 2.20-2.05 (m, 4H), 1.95-1.89 (m, 1H), 1.72-1.58 (m, 8H), 1.46 (d, J=6.8 Hz, 3H), 1.40-1.34 (m, 7H), 0.84-0.82 (m, 3H), 0.68-0.60 (m, 3H).

### Example 12

### (3S)-1-[(2R)-2-chloro-2-fluoro-acetyl]-N-[(1S)-1-[[(8S,14S)-22-ethyl-4-hydroxy-(21M)-21-[2-[(1S)-1-methoxyethyl]-5-(4-methylpiperazin-1-yl)-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-pyrrolidine-3-carboxamide

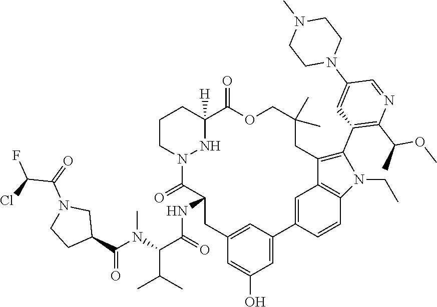

The title compound was prepared in analogy to the preparation of Example 1 by using (2R)-2-chloro-2-fluoro-acetic acid and (8S,14S)-8-amino-22-ethyl-4-hydroxy-(21M)-21-[2-[(1S)-1-methoxyethyl]-5-(4-methylpiperazin-1-yl)-3-pyridyl]-18,18-dimethyl-16-oxa-10,22,28-triazapentacyclo[18.5.2.12,6.110,14.023,27]nonacosa-1(26),2,4,6(29),20,23(27),24-heptaene-9,15-dione (intermediate G) instead of (2S)-2-chloro-2-fluoro-acetic acid (compound 1g) and (8S,14S)-8-amino-22-ethyl-4-hydroxy-(21M)-21-[2-[(1S)-1-methoxyethyl]-3-pyridyl]-18,18-dimethyl-16-oxa-10,22,28-triazapentacyclo[18.5.2.12,6.110,14.023,27]nonacosa-1(26),2,4,6(29),20,23(27),24-heptaene-9,15-dione (intermediate C). Example 12 (17 mg) was obtained as a white solid. MS calc'd 1042.5 (MH⁺), measured 1042.7 (MH⁺). ¹H NMR (400 MHz, Methanol-d4) δ ppm 8.42 (d, J=3.2 Hz, 1H), 8.04-8.00 (m, 1H), 7.62-7.58 (m, 1H), 7.49 (dd, J=2.8, 8.8 Hz, 1H), 7.41-7.34 (m, 2H), 7.09-7.04 (m, 1H), 6.90-6.73 (m, 1H), 6.57-6.47 (m, 1H), 5.64-5.55 (m, 1H), 4.72 (d, J=10.4 Hz, 1H), 4.59 (s, 2H), 4.46 (d, J=13.2 Hz, 1H), 4.26-4.17 (m, 2H), 4.12-4.07 (m, 1H), 3.85-3.76 (m, 3H), 3.73-3.64 (m, 3H), 3.60-3.54 (m, 1H), 3.39 (t, J=4.4 Hz, 4H), 3.22-3.14 (m, 3H), 2.97-2.91 (m, 4H), 2.84-2.79 (m, 2H), 2.78-2.73 (m, 5H), 2.44 (s, 3H), 2.40-2.27 (m, 1H), 2.24-2.07 (m, 3H), 2.02-1.91 (m, 2H), 1.74-1.60 (m, 2H), 1.43 (d, J=6.4 Hz, 3H), 1.11-1.05 (m, 3H), 0.94 (t, J=6.4 Hz, 3H), 0.86-0.80 (m, 5H), 0.73-0.64 (m, 3H).

### Example 13

### N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-3-[2-(2-pyridyl)ethynyl]cyclobutanecarboxamide

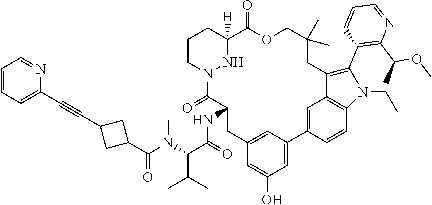

The compound was prepared according to the following scheme:

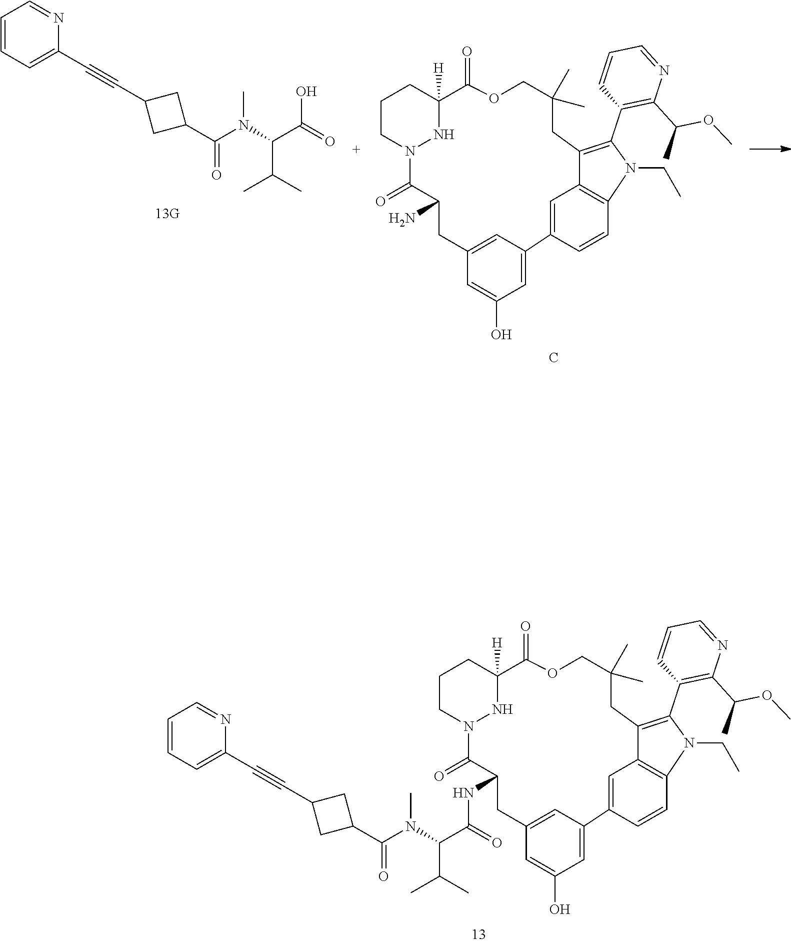

To a solution of (8S,14S)-8-amino-22-ethyl-4-hydroxy-(21M)-21-[2-[(1S)-1-methoxyethyl]-3-pyridyl]-18,18-dimethyl-16-oxa-10,22,28-triazapentacyclo[18.5.2.12,6.110,14.023,27]nonacosa-1(26),2,4,6(29),20,23(27),24-heptaene-9,15-dione (intermediate C, 40 mg, 0.06 mmol) in DMF (3 mL) was added (2S)-3-methyl-2-[methyl-[3-[2-(2-pyridyl)ethynyl]cyclobutanecarbonyl]amino]butanoic acid (compound 13G, 20 mg, 0.06 mmol), T₃P (60 mg, 0.1 mmol) and followed by addition of DIEA (41 mg, 0.32 mmol) slowly at 0° C. After being stirred at 20° C. for another 2 hours, the mixture was diluted with water (15 mL) and then extracted with EtOAc (15 mL, twice). The combined organic phase was washed with brine (15 mL), dried over Na₂SO₄, filtered and concentrated under vacuum to give the residue. The residue was purified by prep-HPLC to afford N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-3-[2-(2-pyridyl)ethynyl]cyclobutanecarboxamide (Example 13, 10.9 mg) as white solid. MS calc'd 936.5 (MH⁺), measured 936.6 (MH⁺). ¹H NMR (400 MHz, METHANOL-d₄) δ ppm 8.73 (dd, J=4.8, 1.6 Hz, 1H), 8.53-8.43 (m, 1H) 8.17-8.13 (m, 0.3H), 8.03-7.97 (m, 1H), 7.94-7.87 (m, 1H), 7.85-7.77 (m, 1H), 7.62-7.46 (m, 4H), 7.43-7.33 (m, 2H), 7.09-6.99 (m, 1H), 6.65-6.48 (m, 1H), 5.66-5.53 (m, 1H) 4.74-4.63 (m, 1H), 4.50-4.39 (m, 1H), 4.36-4.27 (m, 1H), 4.26-4.16 (m, 1H), 4.09-3.98 (m, 1H), 3.88-3.72 (m, 2H), 3.70-3.53 (m, 2H), 3.27-3.19 (m, 3H), 3.18-2.96 (m, 2H), 2.96-2.72 (m, 7H), 2.70-2.59 (m, 2H), 2.54-2.33 (m, 2H), 2.22-2.05 (m, 2H), 1.95-1.81 (m, 1H), 1.74-1.55 (m, 2H), 1.46 (d, J=6.0 Hz, 3H), 1.10-1.02 (m, 3H), 0.95 (dd, J=6.4, 3.2 Hz, 1.5H), 0.89-0.85 (m, 1.5H), 0.85-0.71 (m, 6H), 0.64 (s, 3H).

The compound 13G was prepared according to the following scheme:

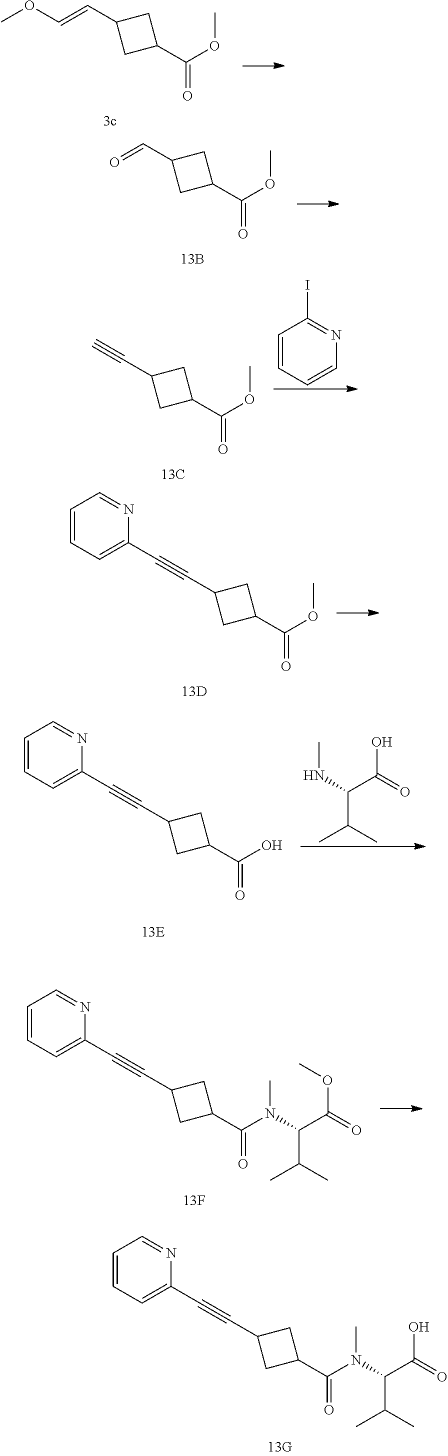

### Step 1: Preparation of methyl 3-formylcyclobutanecarboxylate (compound 13B)

To a solution of methyl 3-(methoxymethylene)cyclobutanecarboxylate (compound 3c, 3.5 g, 22.41 mmol) in DCM (100 mL) and water (10 mL) was added trifluoroacetic acid (5.1 g, 44.82 mmol). After being stirred at 20° C. for 3 hours, the reaction mixture was added H₂O (60 mL) and then extracted with DCM (50 mL, three times). The organic phase was washed with brine (80 mL), dried over Na₂SO₄, filtered and concentrated under vacuum to afford methyl 3-formylcyclobutanecarboxylate (compound 13B, 3 g) as colorless oil. ¹H NMR (400 MHz, CDCl3) δ ppm 9.82-9.68 (m, 1H), 3.72-3.69 (m, 3H), 3.26-3.07 (m, 2H), 2.56-2.39 (m, 4H)

### Step 2: Preparation of methyl 3-ethynylcyclobutanecarboxylate (compound 13C)

To a solution of methyl 3-oxocyclobutanecarboxylate (compound 13B, 3.0 g, 23.41 mmol) in methanol (30 mL) cooled to 0° C. was added dimethyl (1-diazo-2-oxopropyl)phosphonate (7.2 g, 37.46 mmol) and potassium carbonate (6.47 g, 46.83 mmol). The resulting mixture was stirred at 0° C. for 1 hour then warmed up to 20° C. for 3 hours. The reaction mixture was quenched with water (80 mL) then extracted with PE (60 mL, twice). The combined organic phase was washed with brine (80 mL), dried over Na₂SO₄, filtered and concentrated under vacuum to give the residue. The residue was purified by silica gel chromatography eluting with PE to afford methyl 3-ethynylcyclobutanecarboxylate (compound 13C, 1.5 g) as colorless oil. ¹H NMR (400 MHz, CDCl3) δ ppm 3.71-3.66 (m, 3H), 3.34-3.10 (m, 1H), 3.08-2.89 (m, 1H), 2.63-2.49 (m, 2H), 2.47-2.32 (m, 2H), 2.23-2.16 (m, 1H).

### Step 3: Preparation of methyl 3-[2-(2-pyridyl)ethynyl]cyclobutanecarboxylate (compound 13D)

To a solution of methyl 3-ethynylcyclobutanecarboxylate (compound 13C, 500 mg, 3.62 mmol) in THF (5 mL) was added TEA (732 mg, 7.24 mmol), methyl 3-ethynylcyclobutanecarboxylate (500 mg, 3.62 mmol), tetrakis(triphenylphosphine)palladium(0) (418 mg, 0.360 mmol) and copper(I) iodide (68 mg, 0.36 mmol). The reaction mixture was degassed and purged with nitrogen for 3 times and stirred at 50° C. for 1 hour. After the reaction was complete, the mixture was diluted with water (60 mL) and then extracted with EtOAc (40 mL, twice). The combined organic phase was washed with brine (60 mL), dried over Na₂SO₄, filtered and concentrated under vacuum to give the residue. The residue was purified by silica gel chromatography (EtOAc in PE=0-50%) to afford methyl 3-[2-(2-pyridyl)ethynyl]cyclobutanecarboxylate (compound 13D, 500 mg) as yellow oil. ¹H NMR (400 MHz, CDCl3) δ ppm 8.55 (d, J=4.4 Hz, 1H), 7.66-7.59 (m, 1H), 7.38 (d, J=7.6 Hz, 1H), 7.23-7.17 (m, 1H), 3.72-3.69 (m, 3H), 3.48-3.26 (m, 1H), 3.25-3.02 (m, 1H), 2.71-2.47 (m, 4H).

### Step 4: Preparation of 3-[2-(2-pyridyl)ethynyl]cyclobutanecarboxylic acid (compound 13E)

To a solution of methyl 3-[2-(2-pyridyl)ethynyl]cyclobutanecarboxylate (compound 13D, 500 mg, 2.32 mmol) in THF (2.5 mL) and water (2.5 mL) was added lithium hydroxide monohydrate (194 mg, 4.65 mmol) slowly. The reaction mixture was stirred at 20° C. for 2 hours. After the reaction was complete, the reaction mixture was concentrated under vacuum to remove THF and then acidified with 1 M aqueous HCl to pH=5. The mixture was extracted with EtOAc (80 mL×2). The combined organic phase was washed with brine (15 mL), dried over Na₂SO₄, filtered and concentrated under vacuum to afford 3-[2-(2-pyridyl)ethynyl]cyclobutanecarboxylic acid (compound 13E, 500 mg) as yellow oil. ¹H NMR (400 MHz, CDCl3) δ ppm 8.58 (d, J=4.4 Hz, 1H), 7.68 (t, J=7.6 Hz, 1H), 7.44-7.37 (m, 1H), 7.25 (d, J=5.6 Hz, 1H), 3.54-3.34 (m, 1H), 3.2-3.10 (m, 1H), 2.71-2.61 (m, 4H).

### Step 5: Preparation of methyl (2S)-3-methyl-2-[methyl-[3-[2-(2-pyridyl)ethynyl]cyclobutanecarbonyl]amino]butanoate (compound 13F)

To a solution of 3-[2-(2-pyridyl)ethynyl]cyclobutanecarboxylic acid (compound 13E, 100 mg, 0.500 mmol) in DMF (3 mL) was added DIEA (192.7 mg, 1.49 mmol) and HATU (207 mg, 0.55 mmol). After being stirred at 20° C. for 15 min, methyl (2S)-3-methyl-2-(methylamino)butanoate;hydrochloride (99 mg, 0.55 mmol) was added. The reaction mixture was stirred at 20° C. for 12 hours. After the reaction was complete, the mixture was purified by reversed phase chromatography afford methyl (2S)-3-methyl-2-[methyl-[3-[2-(2-pyridyl)ethynyl]cyclobutanecarbonyl]amino]butanoate (compound 13F, 160 mg) as yellow oil. MS calc'd 329.2 (MH⁺), measured 329.3 (MH⁺)

### Step 6: Preparation of (2S)-3-methyl-2-[methyl-[3-[2-(2-pyridyl)ethynyl]cyclobutanecarbonyl]amino]butanoic acid (compound 13G)

To a solution of methyl (2S)-3-methyl-2-[methyl-[3-[2-(2-pyridyl)ethynyl]cyclobutanecarbonyl]amino]butanoate (compound 13F, 80 mg, 0.24 mmol) in water (2 mL) and THF (2 mL) was added lithium hydroxide monohydrate (19 mg, 0.49 mmol). The reaction mixture was stirred at 20° C. for 2 hours. After the reaction was complete, the mixture was concentrated under vacuum to remove THF and the aqueous was acidified with 1N HCl aqueous to pH=5. The resulting mixture was extracted with EtOAc (15 mL, twice) and the combined organic phase was washed with brine (20 mL), dried over Na₂SO₄, filtered and concentrated under vacuum to afford (2S)-3-methyl-2-[methyl-[3-[2-(2-pyridyl)ethynyl]cyclobutanecarbonyl]amino]butanoic acid (compound 13G, 60 mg) as yellow oil. MS calc'd 315.2 (MH⁺), measured 315.2 (MH⁺).

### Example 14

### N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-3-ethynyl-N-methyl-azetidine-1-carboxamide

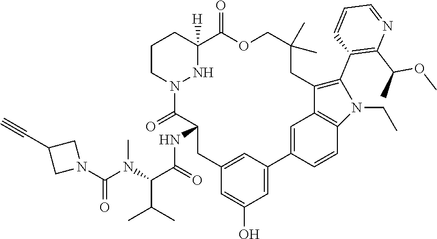

The title compound was prepared in analogy to the preparation of Example 13 by using 3-ethynylazetidine instead of 3-[2-(2-pyridyl)ethynyl]cyclobutanecarboxylic acid (compound 13E). Example 14 (4 mg) was obtained as a white solid. MS calc'd 860.5 (MH⁺), measured 860.4 (MH⁺). ¹H NMR (400 MHz, MeOD) δ ppm 8.74-8.73 (m, 1H), 8.0 (d, J=13.2 Hz, 1H), 7.90 (d, J=8 Hz, 1H), 7.60-7.49 (m, 3H), 7.36 (d, J=7.2 Hz, 1H), 7.04 (s, 1H), 6.54 (s, 1H), 5.66-5.63 (m, 1H), 4.32-4.27 (m, 1H), 4.21-4.18 (m, 1H), 4.07-3.95 (m, 2H), 3.77 (s, 1H), 3.66-3.42 (m, 2H), 3.00-2.94 (m, 4H), 2.76 (s, 3H), 2.69 (s, 3H), 2.37-2.33 (t, J=7.2 Hz, 1H), 2.15-2.10 (m, 2H), 2.04-2.02 (m, 1H), 1.94-1.90 (m, 1H), 1.69-1.57 (m, 4H), 1.47-1.45 (d, 6.0 Hz, 3H) 1.29-1.28 (m, 4H), 1.07-1.03 (m, 3H), 0.93-0.83 (m, 9H), 0.64 (s, 3H).

### Example 15

### N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-4-ethynyl-N-methyl-piperidine-1-carboxamide

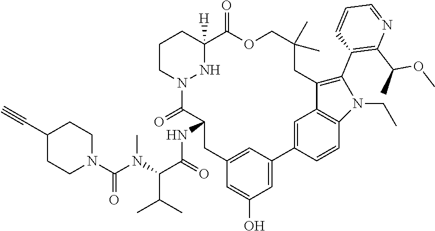

The title compound was prepared in analogy to the preparation of Example 13 by using 4-ethynylpiperidine instead of 3-[2-(2-pyridyl)ethynyl]cyclobutanecarboxylic acid (compound 13E). Example 15 (5 mg) was obtained as a white solid. MS calc'd 888.5 (MH⁺), measured 888.6 (MH⁺). ¹H NMR (400 MHz, DMSO-d₆) δ ppm 9.30 (s, 1H), 8.78-8.72 (m, 1H), 8.48 (s, 1H), 8.14-8.06 (m, 1H), 7.91 (s, 1H), 7.86-7.78 (m, 1H), 7.63-7.57 (m, 1H), 7.56-7.48 (m, 2H), 7.32-7.23 (m, 1H), 7.06-6.98 (m, 1H), 6.40-6.32 (m, 1H), 5.51-5.41 (m, 1H), 5.38-5.29 (m, 1H), 4.33-4.16 (m, 4H), 4.09-3.96 (m, 2H), 3.89-3.78 (m, 3H), 3.68-3.62 (m, 2H), 3.63-3.52 (m, 2H), 3.12 (s, 3H), 3.07-2.89 (m, 3H), 2.87-2.74 (m, 2H), 2.69 (s, 1H), 2.61 (s, 2H), 2.10-1.92 (m, 2H), 1.87-1.74 (m, 3H), 1.69-1.56 (m, 2H), 1.55-1.46 (m, 2H), 1.42-1.35 (m, 3H), 1.00-0.92 (m, 3H), 0.83 (d, J=6.4 Hz, 3H), 0.80-0.74 (m, 6H), 0.58-0.46 (m, 3H).

### Example 16

### N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-4-ethynyl-4-fluoro-N-methyl-piperidine-1-carboxamide

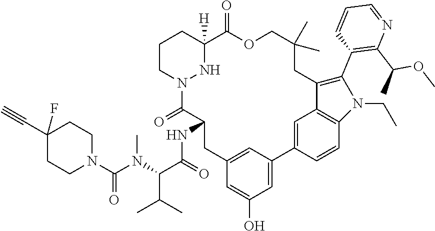

The title compound was prepared in analogy to the preparation of Example 13 by using 4-ethynyl-4-fluoropiperidine instead of 3-[2-(2-pyridyl)ethynyl]cyclobutanecarboxylic acid (compound 13E). Example 16 (6 mg) was obtained as a white solid. MS calc'd 906.5 (MH⁺), measured 906.3 (MH⁺). ¹H NMR (400 MHz, DMSO-d6) δ ppm 9.33 (s, 1H), 8.79-8.73 (m, 1H), 8.45 (s, 1H), 8.14-8.05 (m, 1H), 7.97-7.92 (m, 1H), 7.86-7.79 (m, 1H), 7.65-7.49 (m, 3H), 7.21 (s, 1H), 7.06-6.95 (m, 1H), 6.40-6.31 (m, 1H), 5.55-5.25 (m, 2H), 4.34-4.18 (m, 4H), 4.08-3.77 (m, 9H), 3.71-3.61 (m, 4H), 3.13-3.05 (m, 3H), 3.02-2.71 (m, 1H), 2.63-2.60 (m, 3H), 2.12-1.91 (m, 6H), 1.42-1.36 (m, 3H), 1.26-1.13 (m, 2H), 1.00-0.91 (m, 3H), 0.87-0.80 (m, 4H), 0.80-0.73 (m, 6H), 0.60-0.43 (m, 3H).

### Example 17

### (3S)-1-[(2R)-2-chloro-2-fluoro-acetyl]-N-[(1S)-1-[[(7S,13S)-21-ethyl-(20M)-20-[2-[(1S)-1-methoxyethyl]-3-pyridyl]-17,17-dimethyl-8,14-dioxo-15-oxa-4-thia-9,21,27,28-tetrazapentacyclo[17.5.2.12,5.19,13.022,26]octacosa-1(25),2,5(28),19,22(26),23-hexaen-7-yl]carbamoyl]-2-methyl-propyl]-N-methyl-pyrrolidine-3-carboxamide

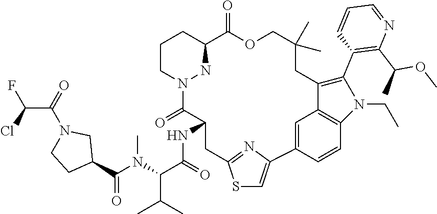

The title compound was prepared in analogy to the preparation of Example 1 by using (2R)-2-chloro-2-fluoro-acetic acid and (7S,13S)-7-amino-21-ethyl-(20M)-20-[2-[(1S)-1-methoxyethyl]-3-pyridyl]-17,17-dimethyl-15-oxa-4-thia-9,21,27,28-tetrazapentacyclo[17.5.2.12,5.19,13.022,26]octacosa-1(25),2,5(28),19,22(26),23-hexaene-8,14-dione (intermediate H) instead of (2S)-2-chloro-2-fluoro-acetic acid (compound 1g) and (8S,14S)-8-amino-22-ethyl-4-hydroxy-(21M)-21-[2-[(1S)-1-methoxyethyl]-3-pyridyl]-18,18-dimethyl-16-oxa-10,22,28-triazapentacyclo[18.5.2.12,6.110,14.023,27]nonacosa-1(26),2,4,6(29),20,23(27),24-heptaene-9,15-dione (intermediate C). Example 17 (47 mg) was obtained as a white solid. MS calc'd 935.4 (MH⁺), measured 935.6 (MH⁺). ¹H NMR (400 MHz, MeOD) δ ppm 8.77-8.68 (m, 1H), 8.60-8.56 (m, 1H), 7.89-7.80 (m, 1H), 7.76-7.66 (m, 1H), 7.59-7.43 (m, 3H), 6.95-6.73 (m, 1H), 5.85-5.67 (m, 1H), 4.84-4.77 (m, 2H), 4.47-4.34 (m, 2H), 4.33-4.06 (m, 3H), 3.98-3.66 (m, 6H), 3.63-3.39 (m, 2H), 3.38-3.33 (m, 3H), 3.30-3.20 (m, 1H), 3.14-2.99 (m, 4H), 2.86-2.74 (m, 1H), 2.64-2.53 (m, 1H), 2.38-2.02 (m, 4H), 1.99-1.90 (m, 1H), 1.87-1.72 (m, 1H), 1.69-1.54 (m, 1H), 1.45 (d, J=6.0 Hz, 3H), 1.02-0.96 (m, 3H), 0.95-0.92 (m, 5H), 0.91-0.85 (m, 3H), 0.57-0.40 (m, 3H).

### Example 18

### (2S)-2-[2-[(2R)-2-chloro-2-fluoro-acetyl]-6-oxo-2,7-diazaspiro[4.5]decan-7-yl]-N-[(7S,13S)-21-ethyl-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.12,5.19,13.022,26]octacosa-1(25),2,5(28),19,22(26),23-hexaen-7-yl]-3-methyl-butanamide

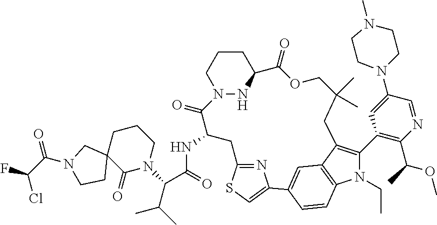

The title compound was prepared in analogy to the preparation of Example 1 by using (2R)-2-chloro-2-fluoro-acetic acid and (7S,13S)-7-amino-21-ethyl-(20M)-20-[2-[(1S)-1-methoxyethyl]-5-(4-methylpiperazin-1-yl)-3-pyridyl]-17,17-dimethyl-15-oxa-4-thia-9,21,27,28-tetrazapentacyclo[17.5.2.12,5.19,13.022,26]octacosa-1(25),2,5(28),19,22(26),23-hexaene-8,14-dione (intermediate F) and (2S)-2-(2-(tert-butoxycarbonyl)-6-oxo-2,7-diazaspiro[4.5]decan-7-yl)-3-methylbutanoic acid (compound 18l) instead of (2S)-2-chloro-2-fluoro-acetic acid (compound 1g) and (8S,14S)-8-amino-22-ethyl-4-hydroxy-(21M)-21-[2-[(1S)-1-methoxyethyl]-3-pyridyl]-18,18-dimethyl-16-oxa-10,22,28-triazapentacyclo[18.5.2.12,6.110,14.023,27]nonacosa-1(26),2,4,6(29),20,23(27),24-heptaene-9,15-dione (intermediate C) and BOC-N-ME-VAL-OH (compound 1a). Example 18 (25.4 mg) was obtained as a white solid. MS calc'd 1059.5 (MH⁺), measured 1059.4 (MH⁺). ¹H NMR (400 MHz, Methanol-d4) δ=8.59-8.55 (m, 1H), 8.41 (d, J=2.4 Hz, 1H), 7.74-7.68 (m, 1H), 7.62-7.54 (m, 1H), 7.51-7.46 (m, 1H), 7.35-7.31 (m, 1H), 6.94-6.74 (m, 1H), 5.78-5.66 (m, 1H), 4.59-4.39 (m, 2H), 4.32-4.20 (m, 4H), 4.11-3.97 (m, 1H), 3.94-3.86 (m, 1H), 3.81-3.67 (m, 4H), 3.64-3.52 (m, 2H), 3.49-3.41 (m, 3H), 3.39-3.35 (m, 4H), 3.13-3.07 (m, 1H), 2.81-2.59 (m, 7H), 2.46-2.38 (m, 4H), 2.33-2.20 (m, 2H), 1.98-1.76 (m, 8H), 1.67-1.61 (m, 1H), 1.41 (d, J=6.0 Hz, 3H), 1.02-0.95 (m, 9H), 0.91-0.87 (m, 3H), 0.47 (s, 3H) ppm.

The compound 18l was prepared according to the following scheme:

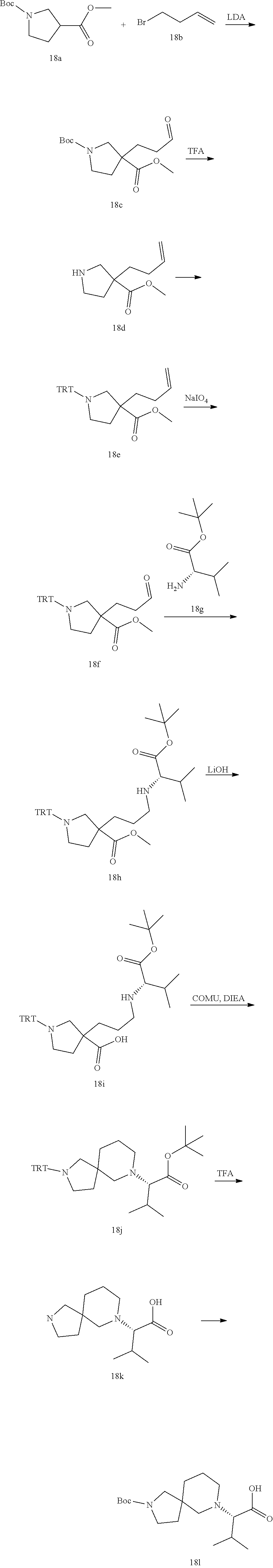

### Step 1: Preparation of 1-(tert-butyl) 3-methyl 3-(but-3-en-1-yl)pyrrolidine-1,3-dicarboxylate (compound 18c)

To a solution of 1-(tert-butyl) 3-methyl pyrrolidine-1,3-dicarboxylate (compound 18a, 25.0 g, 109.04 mmol) in THF (300 mL) was added lithium diisopropylazanide (59.9 mL, 119.95 mmol) at −70° C. under nitrogen atmosphere. After being stirred for 0.5 h, 4-bromo-1-butene (compound 18b, 16.1 g, 119.95 mmol) was added. The mixture was stirred at 20° C. for 2.5 h. The mixture was quenched by addition of saturated NH₄Cl (100 mL) and extracted by EtOAc (150 mL, twice). The organic phase was dried over anhydrous sodium sulfate, filtered and concentrated under reduced pressure to give a residue, which was purified by silica gel column (EtOAc in PE=0%˜5%) and concentrated to afford 1-(tert-butyl) 3-methyl 3-(but-3-en-1-yl)pyrrolidine-1,3-dicarboxylate (compound 18c, 13.2 g) as a yellow oil. ¹H NMR (400 MHz, Methanol-d4) δ=5.83-5.73 (m, 1H), 5.05-4.93 (m, 2H), 3.84 (d, J=10.8 Hz, 1H), 3.71 (s, 3H), 3.46-3.37 (m, 1H), 3.29-3.11 (m, 2H), 2.40-2.32 (m, 1H), 2.01-1.91 (m, 2H), 1.90-1.74 (m, 3H), 1.46 (d, J=3.2 Hz, 9H) ppm.

### Step 2: Preparation of methyl 3-(but-3-en-1-yl)pyrrolidine-3-carboxylate (compound 18d)

To a solution of 1-(tert-butyl) 3-methyl 3-(but-3-en-1-yl)pyrrolidine-1,3-dicarboxylate (compound 18c, 13.2 g, 46.58 mmol) in 1,4-dioxane (50 mL) was added HCl/1,4-dioxane (50.0 mL, 4 M). The mixture was stirred at 20° C. for 0.5 h. The mixture was concentrated under reduced pressure to afford methyl 3-(but-3-en-1-yl)pyrrolidine-3-carboxylate hydrochloride salt (compound 18d, 10.2 g) as yellow oil.

### Step 3: Preparation of methyl 3-(but-3-en-1-yl)-1-tritylpyrrolidine-3-carboxylate (compound 18e)

To a solution of methyl 3-(but-3-en-1-yl)pyrrolidine-3-carboxylate hydrochloride salt (compound 18d, 3.0 g, 13.65 mmol) in ACN (70 mL) was added triphenylmethyl chloride (3.81 g, 13.65 mmol) and potassium carbonate (4.72 g, 34.14 mmol). The mixture was stirred at 20° C. for 12 hrs. After completion of the reaction, the mixture was added into water (100 mL) and extracted with EtOAc (30 mL, three times). The combined organic phase was washed by brine (30 mL), dried over anhydrous sodium sulfate, filtered and concentrated in vacuum to give a residue, which was purified by silica gel column (EtOAc in PE=0%-5%) and concentrated to afford methyl 3-(but-3-en-1-yl)-1-tritylpyrrolidine-3-carboxylate (compound 18e, 1.3 g) as yellow oil. ¹H NMR (400 MHz, CDCl3) δ=7.48-7.45 (m, 6H), 7.26-7.23 (m, 6H), 7.15 (t, J=7.2 Hz, 3H), 5.78-5.68 (m, 1H), 5.00-4.89 (m, 2H), 3.74 (s, 3H), 3.00 (d, J=9.6 Hz, 1H), 2.61-2.55 (m, 1H), 2.43-2.35 (m, 1H), 2.20-2.14 (m, 1H), 2.03 (s, 1H), 1.98-1.83 (m, 2H), 1.82-1.61 (m, 2H), 1.52-1.45 (m, 1H) ppm.

### Step 4: Preparation of methyl 3-(3-oxopropyl)-1-tritylpyrrolidine-3-carboxylate (compound 18f)

To a solution of methyl 3-(but-3-en-1-yl)-1-tritylpyrrolidine-3-carboxylate (compound 18e, 660 mg, 1.55 mmol) in THF (20 mL) and water (10 mL) was added potassium osmate (VI) (5 mg, 0.02 mmol) followed by addition of sodium periodate (663 mg, 3.1 mmol). The mixture was stirred at 20° C. for 2 hrs. After completion of the reaction, the mixture was quenched by addition of sat.NH₄Cl aqueous solution and extracted by EtOAc (50 mL, twice). The organic phase was washed with brine, dried over anhydrous sodium sulfate, filtered and concentrated under reduced pressure to give a residue, which was purified by silica gel column (EtOAc in PE=0%-5%) and concentrated to afford methyl 3-(3-oxopropyl)-1-tritylpyrrolidine-3-carboxylate (compound 18f, 300 mg) as yellow oil. ¹H NMR (400 MHz, CDCl₃) δ=9.75-9.69 (m, 1H), 7.52-7.48 (m, 6H), 7.29 (t, J=4.0 Hz, 6H), 7.19 (t, J=7.2 Hz, 3H), 3.77 (s, 3H), 3.05 (d, J=9.2 Hz, 1H), 2.67-2.57 (m, 1H), 2.47-2.31 (m, 3H), 2.29-2.21 (m, 1H), 2.04-1.87 (m, 2H), 1.62-1.43 (m, 2H) ppm.

### Step 5: Preparation of methyl 3-(3-(((S)-1-(tert-butoxy)-3-methyl-1-oxobutan-2-yl)amino)propyl)-1-tritylpyrrolidine-3-carboxylate (compound 18h)

To a stirred mixture of methyl 3-(3-oxopropyl)-1-tritylpyrrolidine-3-carboxylate (compound 18f, 550.0 mg, 1.29 mmol), H-VAL-OTBU HCl salt (compound 18g, 296.76 mg, 1.42 mmol) and zinc chloride (192.8 mg, 1.42 mmol) in methanol (6 mL) was added sodium cyanoborohydride (88.9 mg, 1.42 mmol) at 0° C. The mixture was stirred at 25° C. for 2 h. After completion of the reaction, water (30 mL) was added to the reaction mixture. The resulting mixture was concentrated under reduced pressure to remove MeOH. The resulting suspension was diluted with water (40 mL) and extracted by EtOAc (30 mL, twice). The organic phase was washed with brined, dried over anhydrous sodium sulfate, filtered and concentrated under reduced pressure to give a residue which was purified by column chromatography (EtOAc in PE=0%˜10%) to afford methyl 3-(3-(((S)-1-(tert-butoxy)-3-methyl-1-oxobutan-2-yl)amino)propyl)-1-tritylpyrrolidine-3-carboxylate (compound 18h, 510 mg) as yellow oil. ¹H NMR (400 MHz, CDCl₃) δ=7.47 (d, J=8.0 Hz, 6H), 7.28-7.27 (m, 1H), 7.27-7.23 (m, 5H), 7.18-7.13 (m, 3H), 3.78-3.73 (m, 3H), 3.05 (t, J=8.4 Hz, 1H), 2.79 (dd, J=2.4, 6.0 Hz, 1H), 2.67-2.59 (m, 1H), 2.55-2.49 (m, 1H), 2.43-2.31 (m, 2H), 2.14-2.09 (m, 1H), 1.99-1.94 (m, 1H), 1.88-1.82 (m, 1H), 1.68-1.64 (m, 3H), 1.60-1.55 (m, 2H), 1.48-1.45 (m, 9H), 1.37-1.33 (m, 1H), 0.94-0.90 (m, 6H) ppm.

### Step 6: Preparation of 3-(3-(((S)-1-(tert-butoxy)-3-methyl-1-oxobutan-2-yl)amino)propyl)-1-tritylpyrrolidine-3-carboxylic acid (compound 18i)

To a solution of methyl 3-(3-(((S)-1-(tert-butoxy)-3-methyl-1-oxobutan-2-yl)amino)propyl)-1-tritylpyrrolidine-3-carboxylate (compound 18h, 510 mg, 0.8 mmol) in methanol (6 mL), water (0.6 mL) and THF (0.6 mL) was added lithium hydroxide monohydrate (336 mg, 8 mmol). The mixture was stirred at 60° C. for 12 hrs. After completion of the reaction, the mixture was diluted with water (20 mL), neutralized with 1 M HCl aqueous and extracted by EtOAc (20 mL, twice). The organic phase was washed with brine, dried over anhydrous sodium sulfate, filtered and concentrated under reduced pressure to afford 3-(3-(((S)-1-(tert-butoxy)-3-methyl-1-oxobutan-2-yl)amino)propyl)-1-tritylpyrrolidine-3-carboxylic acid (compound 18i, 440 mg) as white gum. ¹H NMR (400 MHz, CDCl₃) δ=7.46 (d, J=8.0 Hz, 6H), 7.26-7.19 (m, 6H), 7.16-7.10 (m, 3H), 3.10-2.86 (m, 4H), 2.54-2.45 (m, 2H), 2.37-2.22 (m, 2H), 2.18-2.10 (m, 1H), 2.04-1.97 (m, 1H), 1.68-1.54 (m, 2H), 1.49-1.44 (m, 9H), 1.41-1.35 (m, 2H), 0.99-0.89 (m, 6H) ppm.

### Step 7: Preparation of tert-butyl (2S)-3-methyl-2-(6-oxo-2-trityl-2,7-diazaspiro[4.5]decan-7-yl)butanoate (compound 18j)

To a solution of 3-(3-(((S)-1-(tert-butoxy)-3-methyl-1-oxobutan-2-yl)amino)propyl)-1-tritylpyrrolidine-3-carboxylic acid (compound 18i, 440 mg, 0.77 mmol) in DMF (5 mL) was added DIEA (0.67 mL, 3.85 mmol) and COMU (4-{{[(1-Cyano-2-ethoxy-2-oxoethylidene)amino]oxayl](dimethylamino)methylene]-hexafluorophosphate, CAS 1075198-30-9) (594 mg, 1.39 mmol) at 0° C. The mixture was stirred at 20° C. for 1 h. After completion of the reaction, the mixture was diluted with waster (20 mL) and extracted by EtOAc (20 mL, twice). The combined organic phase was washed with brine, dried over anhydrous sodium sulfate, filtered and concentrated under reduced pressure to give a residue, which was purified by silica gel column (EtOAc in PE=0%˜10%) to afford tert-butyl (2S)-3-methyl-2-(6-oxo-2-trityl-2,7-diazaspiro[4.5]decan-7-yl)butanoate (compound 18j, 420 mg) as yellow oil. MS calc'd 553.3 (MH⁺), measured 553.2 (MH⁺).

### Step 8: Preparation of (2S)-3-methyl-2-(6-oxo-2,7-diazaspiro[4.5]decan-7-yl)butanoic acid (compound 18k)

To a solution of tert-butyl (2S)-3-methyl-2-(6-oxo-2-trityl-2,7-diazaspiro[4.5]decan-7-yl)butanoate (compound 18j, 550 mg, 1 mmol) in DCM (3 mL) was added TFA (3.0 mL). The mixture was stirred at 20° C. for 12 hrs. After completion of the reaction, the mixture was concentrated under reduced pressure to give a residue, which was dissolved in water (10 mL) and extracted with EtOAc (20 mL, twice). The water phase was concentrated in vacuum to afford (2S)-3-methyl-2-(6-oxo-2,7-diazaspiro[4.5]decan-7-yl)butanoic acid with TFA salt form (compound 18k, 365 mg) as colorless oil. MS calc'd 255.2 (MH⁺), measured 255.2 (MH⁺)

### Step 9: Preparation of (2S)-2-(2-(tert-butoxycarbonyl)-6-oxo-2,7-diazaspiro[4.5]decan-7-yl)-3-methylbutanoic acid (compound 18l)

To a solution of (2S)-3-methyl-2-(6-oxo-2,7-diazaspiro[4.5]decan-7-yl)butanoic acid with TFA salt form (compound 18k, 365 mg, 0.99 mmol) in THF (10 mL) and water (10 mL) was added sodium carbonate (210 mg, 1.98 mmol) and di-t-butyldicarbonate (259 mg, 1.19 mmol). The mixture was stirred at 20° C. for 2 hrs. After completion of the reaction, the mixture was neutralized with 1 M HCl aq. and extracted with EtOAc (20 mL, twice). The organic phase was washed with brine, dried over sodium sulfate and concentrated in vacuum to afford (2S)-2-(2-(tert-butoxycarbonyl)-6-oxo-2,7-diazaspiro[4.5]decan-7-yl)-3-methylbutanoic acid (compound 18l, 350 mg) as a white solid.

### Example 19

### (2S)-2-[2-[(2R)-2-chloro-2-fluoro-acetyl]-6-oxo-2,7-diazaspiro[4.5]decan-7-yl]-N-[(7S,13S)-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.12,5.19,13.022,26]octacosa-1(25),2,5(28),19,22(26),23-hexaen-7-yl]-3-methyl-butanamide

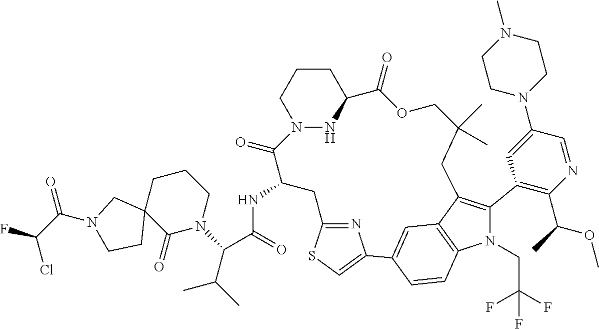

The title compound was prepared in analogy to the preparation of Example 1 by using (2R)-2-chloro-2-fluoro-acetic acid and (7S,13S)-7-amino-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.12,5.19,13.022,26]octacosa-1(25),2,5(28),19,22(26),23-hexaene-8,14-dione (intermediate I) and (2S)-2-(2-(tert-butoxycarbonyl)-6-oxo-2,7-diazaspiro[4.5]decan-7-yl)-3-methylbutanoic acid (compound 18l) instead of (2S)-2-chloro-2-fluoro-acetic acid (compound 1g) and (8S,14S)-8-amino-22-ethyl-4-hydroxy-(21M)-21-[2-[(1S)-1-methoxyethyl]-3-pyridyl]-18,18-dimethyl-16-oxa-10,22,28-triazapentacyclo[18.5.2.12,6.110,14.023,27]nonacosa-1(26),2,4,6(29),20,23(27),24-heptaene-9,15-dione (intermediate C) and BOC-N-ME-VAL-OH (compound 1a). EXAMPLE 19 (54.4 mg) was obtained as a white solid. MS calc'd 1113.5 (MH⁺), measured 1113.3 (MH⁺). ¹H NMR (400 MHz, Methanol-d4) δ=8.61-8.57 (m, 1H), 8.44 (d, J=2.4 Hz, 1H), 7.81-7.74 (m, 1H), 7.68-7.57 (m, 2H), 7.34 (s, 1H), 6.95-6.72 (m, 1H), 5.72-5.62 (m, 1H), 5.20-5.10 (m, 1H), 4.99-4.92 (m, 1H), 4.84-4.77 (m, 2H), 4.70-4.56 (m, 1H), 4.45-4.38 (m, 1H), 4.32-4.25 (m, 1H), 4.21-4.15 (m, 1H), 4.11-3.79 (m, 2H), 3.77-3.65 (m, 3H), 3.59-3.53 (m, 1H), 3.51-3.33 (m, 10H), 3.21-3.15 (m, 1H), 2.5-2.71 (m, 5H), 2.61-2.55 (m, 1H), 2.45 (s, 3H), 2.33-2.20 (m, 2H), 2.02-1.78 (m, 7H), 1.68-1.59 (m, 1H), 1.43 (d, J=6.0 Hz, 3H), 1.02 (d, J=6.4 Hz, 1H), 1.00-0.97 (m, 4H), 0.93-0.79 (m, 4H), 0.42 (s, 3H) ppm.

### Example 20

### 1-[(2R)-2-chloro-2-fluoro-acetyl]-4-fluoro-N-[(1S)-1-[[(7S,13S)-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.12,5.19,13.022,26]octacosa-1(25),2,5(28),19,22(26),23-hexaen-7-yl]carbamoyl]-2-methyl-propyl]-N-methyl-piperidine-4-carboxamide

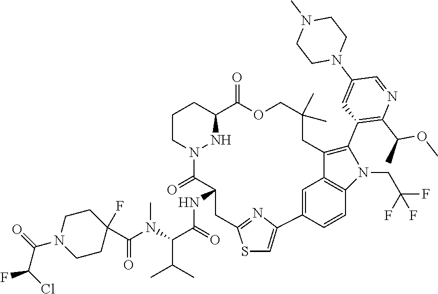

The title compound was prepared in analogy to the preparation of Example 1 by using (2R)-2-chloro-2-fluoro-acetic acid and (7S,13S)-7-amino-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.12,5.19,13.022,26]octacosa-1(25),2,5(28),19,22(26),23-hexaene-8,14-dione (intermediate I) and 1-BOC-4-fluoro-4-piperidinecarboxylic acid instead of (2S)-2-chloro-2-fluoro-acetic acid (compound 1g) and (8S,14S)-8-amino-22-ethyl-4-hydroxy-(21M)-21-[2-[(1S)-1-methoxyethyl]-3-pyridyl]-18,18-dimethyl-16-oxa-10,22,28-triazapentacyclo[18.5.2.12,6.110,14.023,27]nonacosa-1(26),2,4,6(29),20,23(27),24-heptaene-9,15-dione (intermediate C) and (S)-1-BOC-pyrrolidine-3-carboxylic acid (compound 1d). EXAMPLE 20 (94.41 mg) was obtained as a yellow solid. MS calc'd 1119.5 (MH⁺), measured 1119.5 (MH⁺). ¹H NMR (400 MHz, METHANOL-d4) 6=8.64-8.58 (m, 1H), 8.52-8.49 (m, 1H), 7.85-7.75 (m, 1H), 7.65-7.60 (m, 2H), 7.54-7.50 (m, 1H), 7.18-6.96 (m, 1H), 5.82-5.61 (m, 1H), 5.33-5.10 (m, 2H), 4.54-4.36 (m, 2H), 4.33-4.20 (m, 2H), 4.16-3.87 (m, 4H), 3.83-3.66 (m, 1H), 3.85-3.62 (m, 4H), 3.60-3.43 (m, 4H), 3.27-3.19 (m, 6H), 3.10-3.05 (m, 1H), 3.03-2.99 (m, 3H), 2.89-2.76 (m, 1H), 2.63-2.50 (m, 1H), 2.39-2.11 (m, 6H), 2.05-1.93 (m, 1H), 1.91-1.76 (m, 1H), 1.49-1.38 (m, 4H), 1.35-1.28 (m, 1H), 1.15-1.07 (m, 1H), 1.05-0.96 (m, 7H), 0.94-0.85 (m, 3H), 0.51-0.34 (m, 3H).

### Example 21

### (3S)-1-[(2R)-2-chloro-2-fluoro-acetyl]-N-[(1S)-1-[[(7S,13S)-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.12,5.19,13.022,26]octacosa-1(25),2,5(28),19,22(26),23-hexaen-7-yl]carbamoyl]-2-methyl-propyl]-N-methyl-pyrrolidine-3-carboxamide

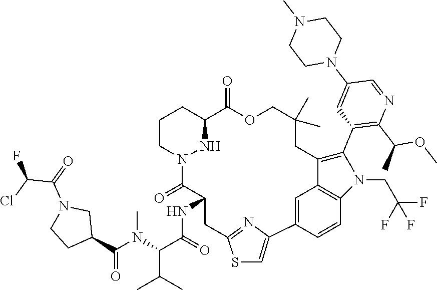

The title compound was prepared in analogy to the preparation of Example 1 by using (2R)-2-chloro-2-fluoro-acetic acid and (7S,13S)-7-amino-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.12,5.19,13.022,26]octacosa-1(25),2,5(28),19,22(26),23-hexaene-8,14-dione (intermediate I) instead of (2S)-2-chloro-2-fluoro-acetic acid (compound 1g) and (8S,14S)-8-amino-22-ethyl-4-hydroxy-(21M)-21-[2-[(1S)-1-methoxyethyl]-3-pyridyl]-18,18-dimethyl-16-oxa-10,22,28-triazapentacyclo[18.5.2.12,6.110,14.023,27]nonacosa-1(26),2,4,6(29),20,23(27),24-heptaene-9,15-dione (intermediate C). Example 21(85.7 mg) was obtained as a white solid. MS calc'd 1087.5 (MH⁺), measured 1087.3 (MH⁺). ¹H NMR (400 MHz, MeOD)=8.62-8.58 (m, 1H), 8.52-8.48 (m, 1H), 7.82-7.74 (m, 1H), 7.66-7.57 (m, 2H), 7.53 (br d, J=3.4 Hz, 1H), 6.94-6.76 (m, 1H), 5.79-5.61 (m, 1H), 5.28-5.12 (m, 1H), 4.97-4.88 (m, 1H), 4.84-4.78 (m, 1H), 4.4-4.38 (m, 1H), 4.32-4.21 (m, 2H), 4.12-3.93 (m, 2H), 3.90-3.56 (m, 9H), 3.54-3.33 (m, 6H), 3.30-3.16 (m, 4H), 3.15-3.09 (m, 3H), 3.01-2.97 (m, 3H), 2.86-2.75 (m, 1H), 2.61-2.50 (m, 1H), 2.46-2.16 (m, 4H), 2.01-1.92 (m, 1H), 1.88-1.75 (m, 1H), 1.70-1.57 (m, 1H), 1.45 (d, J=6.0 Hz, 3H), 1.10-0.96 (m, 6H), 0.95-0.85 (m, 3H), 0.50-0.38 (m, 3H).

### Example 22

### (3S)-1-[(2R)-2-chloro-2-fluoro-acetyl]-N-[(1S)-1-[[(7S,13S)-(20M)-20-[2-[(1S)-1-methoxyethyl]-3-pyridyl]-17,17-dimethyl-8,14-dioxo-21-(2,2,2-trifluoroethyl)-15-oxa-4-thia-9,21,27,28-tetrazapentacyclo[17.5.2.12,5.19,13.022,26]octacosa-1(25),2,5(28),19,22(26),23-hexaen-7-yl]carbamoyl]-2-methyl-propyl]-N-methyl-pyrrolidine-3-carboxamide

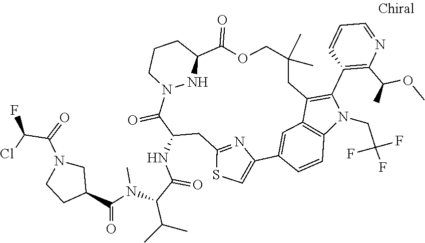

The title compound was prepared in analogy to the preparation of Example 1 by using (2R)-2-chloro-2-fluoro-acetic acid and (7S,13S)-7-amino-(20M)-20-[2-[(1S)-1-methoxyethyl]-3-pyridyl]-17,17-dimethyl-21-(2,2,2-trifluoroethyl)-15-oxa-4-thia-9,21,27,28-tetrazapentacyclo[17.5.2.12,5.19,13.022,26]octacosa-1(25),2,5(28),19,22(26),23-hexaene-8,14-dione (compound 22a) instead of (2S)-2-chloro-2-fluoro-acetic acid (compound 1g) and (8S,14S)-8-amino-22-ethyl-4-hydroxy-(21M)-21-[2-[(1S)-1-methoxyethyl]-3-pyridyl]-18,18-dimethyl-16-oxa-10,22,28-triazapentacyclo[18.5.2.12,6.110,14.023,27]nonacosa-1(26),2,4,6(29),20,23(27),24-heptaene-9,15-dione (intermediate C). Example 22 (66.5 mg) was obtained as an off-white solid. MS calc'd 989.3 (MH⁺), measured 989.4 (MH⁺). ¹H NMR (400 MHz, MeOD) δ=8.80 (dd, J=4.8, 1.2, 1H), 8.74-8.47 (m, 2H), 8.02 (d, J=7.6 Hz, 1H), 7.85-7.74 (m, 1H), 7.73-7.67 (m, 1H), 7.66-7.53 (m, 2H), 6.95-6.74 (m, 1H), 5.88-5.60 (m, 1H), 5.31-5.15 (m, 1H), 4.83-4.78 (m, 1H), 4.47-4.32 (m, 2H), 4.31-4.20 (m, 1H), 4.14-3.86 (m, 1H), 3.85-3.65 (m, 5H), 3.63-3.52 (m, 1H), 3.51-3.42 (m, 1H), 3.42-3.36 (m, 3H), 3.30-3.23 (m, 1H), 3.23-3.14 (m, 1H), 3.13-2.99 (m, 3H), 2.90-2.73 (m, 1H), 2.67-2.50 (m, 1H), 2.42-2.03 (m, 4H), 2.00-1.90 (m, 1H), 1.89-1.72 (m, 1H), 1.70-1.54 (m, 1H), 1.47 (d, J=6.4 Hz, 3H), 1.18-0.99 (m, 3H), 0.98-0.94 (m, 3H), 0.94-0.84 (m, 3H), 0.51-0.35 (m, 3H) ppm.

The compound 22a was prepared in analogy to the preparation of Intermediate H by using CF₃CH₂OTf instead of iodoethane.

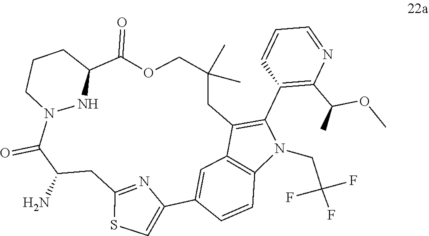

### Example 23 and Example 24

### (2S)-2-[(5S)-7-[(2R)-2-chloro-2-fluoro-acetyl]-1-oxo-2,7-diazaspiro[4.4]nonan-2-yl]-N-[(7S,13S)-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.12,5.19,13.022,26]octacosa-1(25),2,5(28),19,22(26),23-hexaen-7-yl]-3-methyl-butanamide (Example 23)

### (2S)-2-[(5R)-7-[(2R)-2-chloro-2-fluoro-acetyl]-1-oxo-2,7-diazaspiro[4.4]nonan-2-yl]-N-[(7S,13S)-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.12,5.19,13.022,26]octacosa-1(25),2,5(28),19,22(26),23-hexaen-7-yl]-3-methyl-butanamide (Example 24)

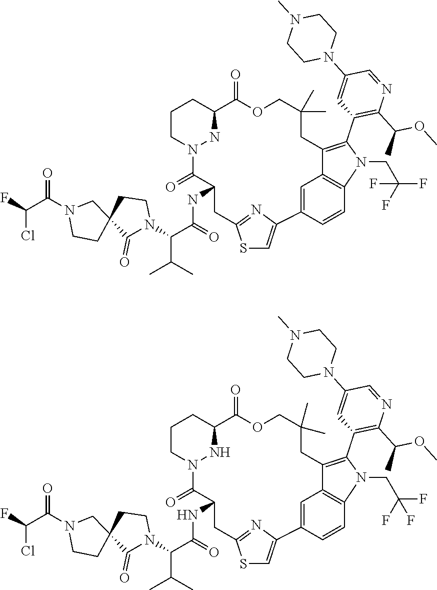

The title compound was prepared in analogy to the preparation of Example 1 by using (2R)-2-chloro-2-fluoro-acetic acid and (7S,13S)-7-amino-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.12,5.19,13.022,26]octacosa-1(25),2,5(28),19,22(26),23-hexaene-8,14-dione (intermediate I) and compound 23g1/compound 23g2 instead of (2S)-2-chloro-2-fluoro-acetic acid (compound 1g) and (8S,14S)-8-amino-22-ethyl-4-hydroxy-(21M)-21-[2-[(1S)-1-methoxyethyl]-3-pyridyl]-18,18-dimethyl-16-oxa-10,22,28-triazapentacyclo[18.5.2.12,6.110,14.023,27]nonacosa-1(26),2,4,6(29),20,23(27),24-heptaene-9,15-dione (intermediate C) and BOC-N-ME-VAL-OH (compound 1a).

Example 23 (21.1 mg) was obtained as a white solid. MS calc'd 1099.5 (MH⁺), measured 1099.4 (MH⁺). ¹H NMR (400 MHz, Methanol-d4) δ=8.59-8.55 (m, 1H), 8.41 (d, J=2.4 Hz, 1H), 7.74-7.68 (m, 1H), 7.63 (d, J=4.4 Hz, 1H), 7.58 (d, J=8.8 Hz, 1H), 7.51-7.46 (m, 1H), 7.35-7.31 (m, 1H), 6.94-6.74 (m, 1H), 5.78-5.66 (m, 1H), 4.59-4.39 (m, 2H), 4.41-4.25 (m, 3H), 4.20-4.14 (m, 1H), 3.79-3.61 (m, 6H), 3.55-3.40 (m, 5H), 3.34-3.34 (m, 3H), 3.21-3.12 (m, 2H), 2.81-2.79 (m, 1H), 2.68-2.58 (m, 5H), 2.36 (s, 3H), 2.28-2.07 (m, 6H), 2.05-1.95 (m, 2H), 1.90-1.77 (m, 1H), 1.73-1.58 (m, 1H), 1.43 (d, J=6.0 Hz, 3H), 1.02 (d, J=6.4 Hz, 3H), 0.99 (s, 3H), 0.90 (d, J=6.4 Hz, 3H), 0.43 (s, 3H) ppm

Example 24 (11.5 mg) was obtained as a white solid. MS calc'd 1099.5 (MH⁺), measured 1099.7 (MH⁺). ¹H NMR (400 MHz, METHANOL-d₄) δ=8.60 (s, 1H), 8.43 (d, J=2.8 Hz, 1H), 7.84-7.72 (m, 1H), 7.68 (s, 1H), 7.59 (d, J=10.0 Hz, 1H), 7.33 (s, 1H), 6.97-6.70 (m, 1H), 5.75-5.65 (m, 1H), 5.16 (br dd, J=8.4, 16.0 Hz, 1H), 4.43-4.24 (m, 3H), 4.18 (br d, J=6.0 Hz, 1H), 4.01-3.89 (m, 1H), 3.88-3.66 (m, 5H), 3.63-3.56 (m, 2H), 3.52-3.41 (m, 3H), 3.39-3.35 (m, 4H), 3.34-3.31 (m, 3H), 3.30-3.25 (m, 1H), 3.22-3.13 (m, 1H), 2.88-2.77 (m, 1H), 2.75-2.60 (m, 3H), 2.62-2.54 (m, 1H), 2.41 (s, 3H), 2.35-2.19 (m, 3H), 2.17-2.04 (m, 3H), 2.02-1.95 (m, 1H), 1.84 (br d, J=13.2 Hz, 1H), 1.70-1.57 (m, 1H), 1.43 (d, J=6.0 Hz, 3H), 1.03-0.97 (m, 6H), 0.91 (dd, J=3.6, 6.4 Hz, 3H), 0.46-0.38 (m, 3H) ppm.

The compound 23g1 and 23g2 were prepared according to the following scheme:

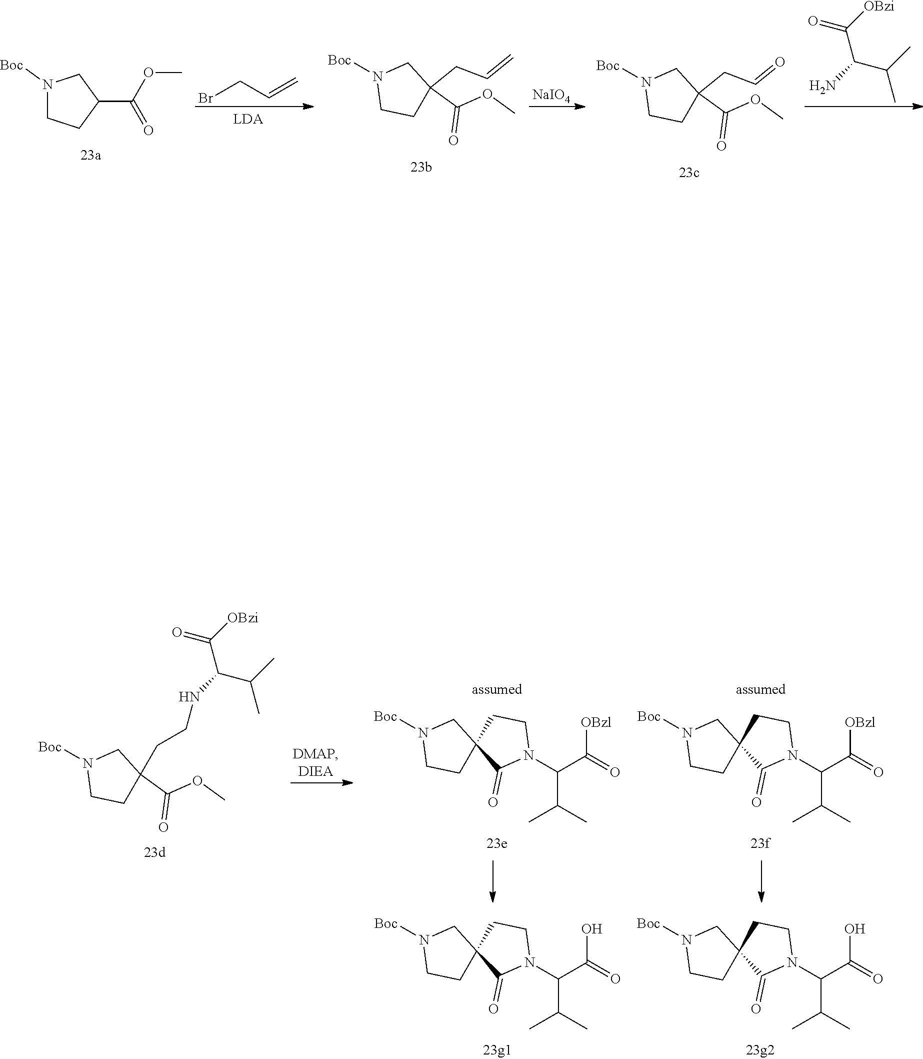

### Step 1: Preparation of 1-(tert-butyl) 3-methyl 3-allylpyrrolidine-1,3-dicarboxylate (compound 23b)

To a solution of 1-(tert-butyl) 3-methyl pyrrolidine-1,3-dicarboxylate (compound 23a, 5.0 g, 21.8 mmol) in THF (60 mL) was added LDA (12 mL, 24 mmol) drop-wise at −70° C. under nitrogen atmosphere. After being stirred for 0.5 h, allyl bromide (2.9 g, 23.99 mmol) was added slowly. After completion of the reaction, the mixture was poured into saturated NH₄Cl aqueous (100 mL) and extracted by EtOAc (70 mL, twice). The combined organic phase was washed with brine (70 mL), dried over Na₂SO₄, filtered and concentrated under reduced pressure to give a residue, which was purified by silica gel column (EtOAc in PE=1%-20%) to afford 1-(tert-butyl) 3-methyl 3-allylpyrrolidine-1,3-dicarboxylate (compound 23b, 2.91 g) as a colorless oil.

### Step 2: Preparation of 1-(tert-butyl) 3-methyl 3-(2-oxoethyl)pyrrolidine-1,3-dicarboxylate (compound 23c)

To the mixture of 1-(tert-butyl) 3-methyl 3-allylpyrrolidine-1,3-dicarboxylate (compound 23b, 2.1 g, 7.8 mmol) in 1,4-dioxane (60 mL) and water (6 mL) was added 2,6-Lutidine (1.8 mL, 15.6 mmol) and K₂OsO₄ (0.14 g, 0.39 mmol) in one portion at 0° C. After being stirred at 0° C. for 15 min, sodium metaperiodate (6.67 g, 31.19 mmol) was added portion-wise at 0° C. The resulting mixture was warmed to 20° C. and stirred for another 6 hrs. After completion of the reaction, the mixture was quenched with saturated Na₂S₂O₃ aqueous solution (100 mL) and extracted with EtOAc (50 mL, three times). The combined organic phase was washed with brine (50 mL), dried with anhydrous Na₂SO₄, filtered and concentrated in vacuum to afford 1-(tert-butyl) 3-methyl 3-(2-oxoethyl)pyrrolidine-1,3-dicarboxylate (compound 23c, 2.1 g) as yellow oil, which was being used for the next step directly.

### Step 3: Preparation of 1-(tert-butyl) 3-methyl 3-(2-(((S)-1-(benzyloxy)-3-methyl-1-oxobutan-2-yl)amino)ethyl)pyrrolidine-1,3-dicarboxylate (compound 23d)

To the mixture of 1-(tert-butyl) 3-methyl 3-(2-oxoethyl)pyrrolidine-1,3-dicarboxylate (compound 23c, 2.1 g, 7.74 mmol) and benzyl (2S)-2-amino-3-methyl-butanoate (1.6 g, 7.74 mmol) in methanol (20 mL) was added zinc chloride (1.05 g, 7.74 mmol) in one portion at 0° C. After being stirred at 0° C. for 1 h, then to the mixture was added sodium cyanoborohydride (0.97 g, 15.48 mmol) at 0° C. The resulting mixture was stirred at 0° C. for another 2 hrs. After completion of the reaction, the mixture was added into saturated NH₄Cl aqueous solution (40 mL) at 0° C. and extracted with EtOAc (50 mL, three times). The combined organic phase was washed by brine (30 mL, four times), dried over anhydrous sodium sulfate, filtered and concentrated in vacuum to give a residue, which was purified by silica gel column (EtOAc in PE=1%-25%) to afford 1-(tert-butyl) 3-methyl 3-(2-(((S)-1-(benzyloxy)-3-methyl-1-oxobutan-2-yl)amino)ethyl)pyrrolidine-1,3-dicarboxylate (compound 23d, 2.2 g) as yellow oil. MS calc'd 463.3 (MH⁺), measured 463.2 (MH⁺).

### Step 4: Preparation of tert-butyl 7-((S)-1-(benzyloxy)-3-methyl-1-oxobutan-2-yl)-6-oxo-2,7-diazaspiro[4.4]nonane-2-carboxylate (compound 23e&23f)

To the mixture of 1-(tert-butyl) 3-methyl 3-(2-(((S)-1-(benzyloxy)-3-methyl-1-oxobutan-2-yl)amino)ethyl)pyrrolidine-1,3-dicarboxylate (compound 23d, 2.1 g, 4.54 mmol) in toluene (20 mL) was added DIEA (7.9 mL, 45.4 mmol) and DMAP (0.55 g, 4.54 mmol) in one portion. The mixture was heated to 80° C. and stirred for 16 hrs. After completion of the reaction, the mixture was poured into water (30 mL) and extracted with EtOAc (30 mL, three times). The combined organic phase was washed with brine (30 mL), dried with anhydrous Na₂SO₄, filtered and concentrated in vacuum to give a residue, which was purified by reversed phase combi-flash and the eluent was concentrated in vacuum. The residue was further separated by prep-SFC (0.1% NH₃·H₂O-MeOH condition, B %=60%, gradient time=7.5 min) and concentrated in vacuum to afford tert-butyl 7-((S)-1-(benzyloxy)-3-methyl-1-oxobutan-2-yl)-6-oxo-2,7-diazaspiro[4.4]nonane-2-carboxylate to afford tert-butyl 7-((S)-1-(benzyloxy)-3-methyl-1-oxobutan-2-yl)-6-oxo-2,7-diazaspiro[4.4]nonane-2-carboxylate (compound 23e, faster eluted, 521 mg, compound 23f, slower eluted, 525 mg). MS calc'd 453.3 (MNa⁺), measured 453.2 (MNa⁺).

### Step 5: Preparation of (S)-2-((R)-7-(tert-butoxycarbonyl)-1-oxo-2,7-diazaspiro[4.4]nonan-2-yl)-3-methylbutanoic acid (compound 23g1)

To a solution of tert-butyl (R)-7-((S)-1-(benzyloxy)-3-methyl-1-oxobutan-2-yl)-6-oxo-2,7-diazaspiro[4.4]nonane-2-carboxylate (compound 23e, 120 mg, 0.28 mmol) in toluene (2 mL) was added wet palladium (12 mg, 10% wt. on activated carbon). The mixture was degassed and purged with hydrogen for 3 times. The mixture was heated to 35° C. and stirred for 3 hrs under hydrogen atmosphere. After completion of the reaction, the solution was filtered through a pad of celite and the filtrate was concentrated in vacuo to give (S)-2-((R)-7-(tert-butoxycarbonyl)-1-oxo-2,7-diazaspiro[4.4]nonan-2-yl)-3-methylbutanoic acid (compound 23g1, 94 mg) as a white solid. MS calc'd 363.2 (MNa⁺), measured 363.1 (MNa⁺).

### Step 6: Preparation of (S)-2-((S)-7-(tert-butoxycarbonyl)-1-oxo-2,7-diazaspiro[4.4]nonan-2-yl)-3-methylbutanoic acid (compound 23g2)

To a solution of tert-butyl (S)-7-((S)-1-(benzyloxy)-3-methyl-1-oxobutan-2-yl)-6-oxo-2,7-diazaspiro[4.4]nonane-2-carboxylate (compound 23f, 120 mg, 0.28 mmol) in toluene (2 mL) was added wet palladium (12 mg, 10% wt. on activated carbon). The mixture was degassed and purged with hydrogen for 3 times. The mixture was heated to 35° C. and stirred for 3 h under hydrogen atmosphere. After completion of the reaction, the solution was filtered through a pad of celite and the filtrate was concentrated in vacuo to afford (S)-2-((S)-7-(tert-butoxycarbonyl)-1-oxo-2,7-diazaspiro[4.4]nonan-2-yl)-3-methylbutanoic acid (compound 23g2, 79 mg) as a white solid. MS calc'd 363.2 (MNa⁺), measured 363.1 (MNa⁺).

### Example 25

### (3S)-1-[(2R)-2-chloro-2-fluoro-acetyl]-N-[(1S)-1-[[(7S,13S)-(20M)-20-[2-[(1S)-1-methoxyethyl]-5-morpholino-3-pyridyl]-17,17-dimethyl-8,14-dioxo-21-(2,2,2-trifluoroethyl)-15-oxa-4-thia-9,21,27,28-tetrazapentacyclo[17.5.2.12,5.19,13.022,26]octacosa-1(25),2,5(28),19,22(26),23-hexaen-7-yl]carbamoyl]-2-methyl-propyl]-N-methyl-pyrrolidine-3-carboxamide

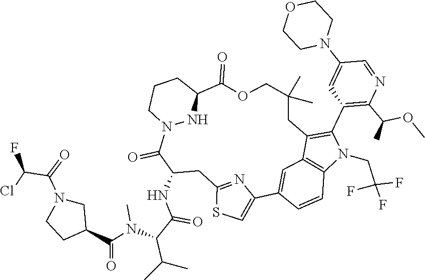

The title compound was prepared in analogy to the preparation of Example 1 by using (2R)-2-chloro-2-fluoro-acetic acid and (7S,13S)-7-amino-(20M)-20-[2-[(1S)-1-methoxyethyl]-5-morpholino-3-pyridyl]-17,17-dimethyl-21-(2,2,2-trifluoroethyl)-15-oxa-4-thia-9,21,27,28-tetrazapentacyclo[17.5.2.12,5.19,13.022,26]octacosa-1(25),2,5(28),19,22(26),23-hexaene-8,14-dione (compound 25a) instead of (2S)-2-chloro-2-fluoro-acetic acid (compound 1g) and (8S,14S)-8-amino-22-ethyl-4-hydroxy-(21M)-21-[2-[(1]S)-1-methoxyethyl]-3-pyridyl]-18,18-dimethyl-16-oxa-10,22,28-triazapentacyclo[18.5.2.12,6.110,14.023,27]nonacosa-1(26),2,4,6(29),20,23(27),24-heptaene-9,15-dione (intermediate C). EXAMPLE 25 (68.8 mg) was obtained as a yellow solid. MS calc'd 1074.4.4 (MH⁺), measured 1074.4 (MH⁺). ¹H NMR (400 MHz, MeOD) δ=8.65-8.60 (m, 1H), 8.45-8.38 (m, 1H), 7.85-7.76 (m, 2H), 7.67-7.57 (m, 2H), 6.94-6.74 (m, 1H), 5.80-5.65 (m, 1H), 5.34-5.22 (m, 1H), 4.88-4.71 (m, 2H), 4.46-4.24 (m, 3H), 3.95-3.58 (m, 12H), 3.45-3.35 (m, 8H), 3.25-3.09 (m, 4H), 2.86-2.76 (m, 1H), 2.64 (dd, J=5.8, 14.4 Hz, 1H), 2.37-2.15 (m, 4H), 2.01-1.92 (m, 1H), 1.88-1.74 (m, 1H), 1.67-1.57 (m, 1H), 1.48 (d, J=6.4 Hz, 3H), 1.04-0.85 (m, 9H), 0.56-0.47 (m, 3H) ppm.

The compound 25a was prepared in analogy to the preparation of Intermediate F and Intermediate D by using CF₃CH₂OTf and morpholine instead of iodoethane and 1-Cbz-piperazine (compound D4).

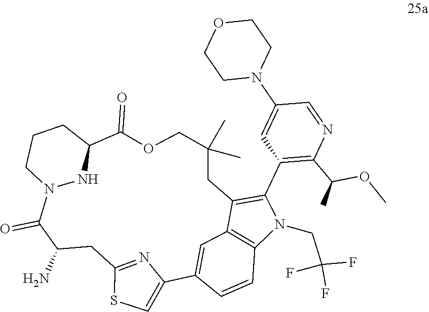

### Example 26

### (3S)-1-[(2R)-2-chloro-2-fluoro-acetyl]-N-[(1S)-1-[[(7S,13S)-21-ethyl-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.12,5.19,13.022,26]octacosa-1(25),2,5(28),19,22(26),23-hexaen-7-yl]carbamoyl]-2-methyl-propyl]-N-methyl-pyrrolidine-3-carboxamide

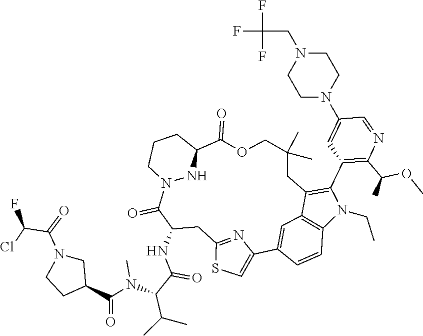

The title compound was prepared in analogy to the preparation of Example 1 by using (2R)-2-chloro-2-fluoro-acetic acid and (7S,13S)-7-amino-21-ethyl-(20M)-20-[2-[(1S)-1-methoxyethyl]-5-[4-(2,2,2-trifluoroethyl)piperazin-1-yl]-3-pyridyl]-17,17-dimethyl-15-oxa-4-thia-9,21,27,28-tetrazapentacyclo[17.5.2.12,5.19,13.022,26]octacosa-1(25),2,5(28),19,22(26),23-hexaene-8,14-dione (compound 26a) instead of (2S)-2-chloro-2-fluoro-acetic acid (compound 1g) and (8S,14S)-8-amino-22-ethyl-4-hydroxy-(21M)-21-[2-[(1S)-1-methoxyethyl]-3-pyridyl]-18,18-dimethyl-16-oxa-10,22,28-triazapentacyclo[18.5.2.12,6.110,14.023,27]nonacosa-1(26),2,4,6(29),20,23(27),24-heptaene-9,15-dione (intermediate C). Example 26 (3.3 mg) was obtained as a white solid. MS calc'd 1101.4 (MH⁺), measured 1101.1 (MH⁺). ¹H NMR (400 MHz, MeOD) δ=8.64-8.37 (m, 3H), 7.79-7.71 (m, 2H), 7.60-7.50 (m, 2H), 6.95-6.74 (m, 1H), 5.90-5.70 (m, 1H), 4.47-4.32 (m, 3H), 4.26-4.09 (m, 2H), 3.97-3.76 (m, 5H), 3.71-3.55 (m, 2H), 3.50-3.43 (s, 5H), 3.43-3.35 (m, 4H), 3.23-3.14 (m, 3H), 3.13-3.09 (m, 3H), 2.92-2.88 (m, 4H), 2.86-2.79 (m, 1H), 2.73-2.65 (m, 1H), 2.39-2.17 (m, 4H), 2.00-1.93 (m, 1H), 1.87-1.76 (m, 1H), 1.71-1.60 (m, 1H), 1.46 (d, J=6.4 Hz, 3H), 1.12-1.03 (m, 2H), 1.02-0.97 (m, 7H), 0.89 (d, J=7.2 Hz, 3H), 0.61-0.53 (m, 3H).

The compound 26a was prepared in analogy to the preparation of Intermediate F by using 1-(2,2,2-trifluoroethyl)piperazine instead of 1-Cbz-piperazine (compound D4).

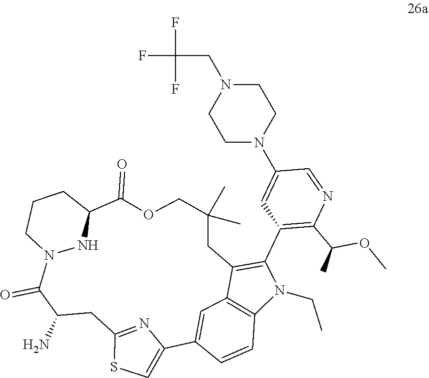

### Biological Example

Compound RM018 (compound A191, page 85) from WO2021091982, compound RM461 (page 115 of FIG. 1) from WO2020132597, and compound RM351 (page 88 of FIG. 1) from WO2020132597 were cited as reference compounds for this invention.

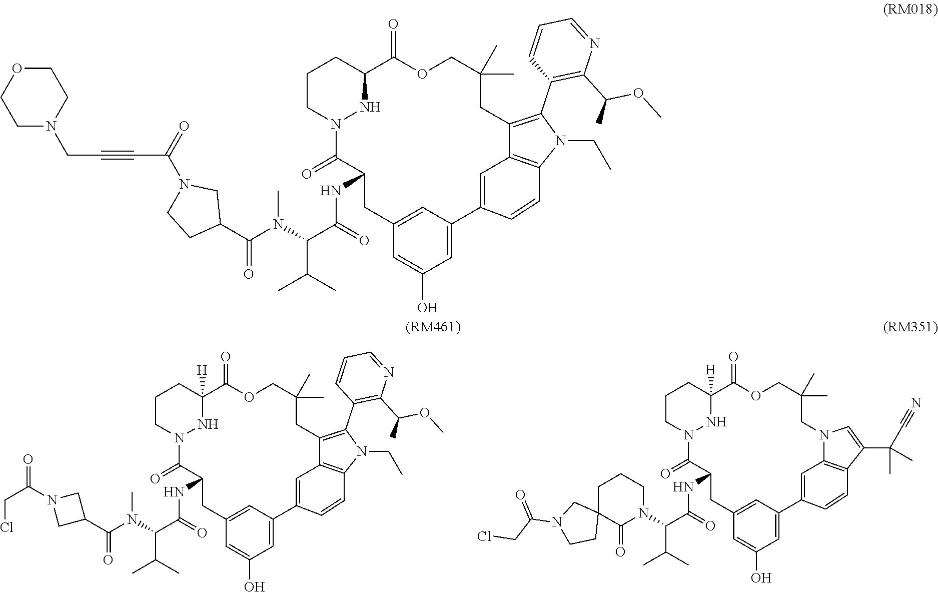

The applicant further synthesized RM461-A with 2-chloro-2-fluoro-acetamide instead of chloroacetamide as the analogue of RM461 according to the procedure described in WO2020132597, and provided the comparison data to demonstrate the improvement and illustrate the technical problem solved.

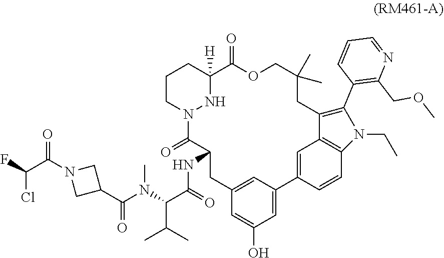 [SMILES: CCn1c(-c2cccnc2COC)c2c3cc(ccc31)-c1cc(O)cc(c1)C[C@H](NC(=O)[C@H](C(C)C)N(C)C(=O)C1CN(C(=O)[C@H](F)Cl)C1)C(=O)N1CCC[C@H](N1)C(=O)OCC(C)(C)C2]

### Example 27

### GSH Reaction Rates

Glutathione (GSH) is a tripeptide found in most of the tissues, especially in high concentrations in the liver, and plays critical roles in protecting cells from oxidative damage and the toxicity of xenobiotic electrophiles, and maintaining redox homeostasis. More specifically, glutathione conjugation helps contribute to detoxification by binding electrophiles that could otherwise bind to proteins or nucleic acids, resulting in cellular damage and genetic mutations.

Many potentially toxic electrophilic xenobiotics and some endogenous compounds are detoxified by conversion to the corresponding glutathione S-conjugate, which consumes inherent GSH and then diminishes detoxification effects. Some drugs and halogenated workplace/environmental contaminants are bioactivated by this mechanism.

On the other hand, conjugation between glutathione and drug molecule in extrahepatic organs as well as in the liver typically leads to the poor PK properties (notably high clearance) of the molecule, and increases its possibility for off-target reactivity (potential liability for various toxicity). Therefore Strategy to minimize the GSH metabolism is very critical. Short T_{1/2} in inherent GSH reaction indicated high GSH reaction rate. Thus T_{1/2} in inherent GSH reaction assay was determined for the screening of candidates.

Reference compounds and compounds of this invention potentially could form conjugation with GSH either through halogenated moieties substitution reaction or direct Michael addition reaction. This test was therefore performed to check GSH reactivity of listed compounds.

For inherent GSH reactivity determination, compounds at 1 μM were incubated at 37° C. with and without 5 mM GSH for 0, 0.5, 1, 2, 4 and 6 h in 100 mM potassium phosphate buffer at pH 7.4. At the end of the designated time points, samples were quenched with acetonitrile containing 10 mM N-ethylmaleimide and an internal standard. Quenched samples were centrifuged, and supernatants were analyzed by LC-MS/MS for compound quantification. If % depletion after 6 hour incubation is less than 20%, compounds were reported as stable; if % depletion is greater than 20%, half-life values are reported.

TABLE 1
GSH Reaction Rates of Examples and
Compounds of present invention

| Example | T_{1/2}(hour) in GSH | Remaining at 6 hr in GSH (%) |
|---|---|---|
| RM461 | 5 | 44 |
| RM351 | 4.3 | 37 |
| RM461-A | stable | 100 |
| Example 10 | stable | 85 |
| Example 19 | stable | 85 |
| Example 21 | stable | 88 |
| Example 23 | stable | 95 |
| Example 25 | stable | 100 |

Above result clearly shows that reference compounds (RM461 and RM351) formed conjugation with GSH causing its depletion over 6 hours while compounds of current invention maintained the stability with much less or no conjugation with GSH. Particularly, comparing reference compound RM461 and its analogue RM461-A, the only difference is that the chloroacetamide was replaced by 2-chloro-2-fluoro-acetamide like the compounds of this invention, surprisingly such small change could address GSH toxicity issue as found in reference compounds.

### Example 28

### KRAS G12C-BRAF NanoBit Assay

This assay is to measure the ability of tested compounds in disruption of the KRAS G12C-BRAF complex at the cellular level, we established the NanoBit cellular assay in mammalian HEK293 (ATCC) cells.

HEK293 cells were grown and maintained using DMEM medium (Thermo Fisher Scientific) with 10% fetal bovine serum and 1% penicillin/streptomycin. Both KRAS G12C and BRAF RBD were cloned into the NanoBit vectors (BiBiT vectors system, Promega) with the orientations SmBit-KRAS G12C and BRAF RBD-LgBit, respectively, and co-transfected into HEK293 cells. Cells were then selected with 100 μg/mL Hygromycin B (10687010, Thermo Fisher) and Blasticidin (5 μg/mL) for 4 weeks to get the stable cell pool.

On the day of the assay, 75 nL of compound solution was presented in a 384-well assay plate as a 16-point 3-fold dilution starting from a final concentration of 30 μM in DMSO. Then cells were seeded at 10,000 cells/25 μL/well in a 384-well plate. After 3 hours of incubation, 6 μL of volume of Nano-Glo® Live Cell Substrate (Promega) was added into each well. Monitor luminescence using ultra384 model in Envision at 20 minutes. Compounds that facilitate disruption of the KRAS G12C-BRAF RBD complex were identified as those eliciting a decrease of luminescence relative to DMSO control wells.

TABLE 2
Activity of Examples and Compounds of present
invention in KRAS G12C-BRAF NanoBit assay

| Example | IC₅₀ (μM) |
|---|---|
| RM018 | 0.268 |
| RM461 | 0.440 |
| RM351 | 1.785 |
| Example 2 | 0.068 |
| Example 10 | 0.021 |
| Example 12 | 0.037 |
| Example 18 | 0.020 |
| Example 19 | 0.021 |
| Example 23 | 0.041 |
| Example 24 | 0.036 |
| Example 25 | 0.020 |

### Example 29

KRAS-BRAF with CYPA (50 nM) Interaction Assay

In this example, TR-FRET was also used to measure the compound or compound-CYPA dependent disruption of the KRAS G12C-BRAF complex. This protocol was also used to measure disruption of KRAS G12D or KRAS G12V binding to BRAF by a compound of the invention, respectively. In assay buffer containing 25 mM HEPES PH=7.4 (4-(2-hydroxyethyl)-1-piperazineethanesulfonic acid, Thermo, 15630080), 0.002% Tween20, 0.1% BSA, 100 mM NaCl, 5 mM MgCl₂, 10 μM GMPPNP (Guanosine 5′-[β,γ-imido]triphosphate trisodium salt hydrate, Sigma, G0635), tagless CYPA, GMPPNP loaded 6His-KRAS proteins, and GST-BRAF^{RBD} were mixed in a well of a 384-well assay plate at final concentrations of 50 nM, 6.25 nM and 1 nM, respectively. Compound was present in plate wells as a 16-point 3-fold dilution series starting at a final concentration of 10 μM and incubated for 3 hours. A mixture of MAb Anti-6His-XL665 (Cisbio, 61HISXLB) and Mab anti-GST-TB cryptate (Cisbio, 61GSTTLB) was then added at a final concentration of 6.67 nM and 0.21 nM, respectively, and the plate was incubated for an additional 1.5 hours. TR-FRET signal was read on a PHERstar FSX microplate reader (Ex320 nm, Em 665/615 nm). Compounds that facilitate disruption of the KRAS-BRAF complex were identified as those eliciting a decrease in the TR-FRET ratio relative to DMSO control wells.

TABLE 3
Activity of Examples and Compounds of present invention
in KRAS-BRAF with CYPA (50 nM) interaction assay

| Example | G12C IC₅₀ (μM) | G12D IC₅₀ (μM) | G12V IC₅₀ (μM) |
|---|---|---|---|
| RM018 | 0.025 | >10 | >10 |
| RM461 | 0.004 | >10 | >10 |
| RM351 | >10 | >10 | >10 |
| Example 2 | 0.006 | 0.298 | 0.025 |
| Example 3 | 0.001 | 1.027 | 0.110 |
| Example 9 | 0.038 | 1.932 | 0.103 |
| Example 12 | 0.002 | 0.006 | 0.003 |
| Example 18 | 0.068 | >10 | >10 |
| Example 20 | 0.011 | 0.415 | 1.166 |
| Example 21 | 0.021 | >10 | >10 |
| Example 23 | 0.021 | >10 | >10 |
| Example 24 | 0.021 | >10 | >10 |
| Example 25 | 0.032 | >10 | >10 |
| Example 26 | 0.036 | 2.721 | 0.200 |

### Example 30

pERK Inhibition Assay

This assay is to measure the ability of test compounds in inhibiting the phosphorylation of ERK, the downstream signaling of KRAS G12C in NCI-H358 cells, KRAS G12D in AGS cells, and KRAS G12V in SW620. NCI-H358 (ATCC-CRL5807) cells, AGS (ATCC-CRL-1739) cells, SW620 (ATCC-CCL-227) cells were all grown and maintained using RPMI-1640 medium (Thermo Fisher Scientific) with 10% fetal bovine serum and 1% penicillin/streptomycin. On the day prior to compound addition, cells were plated in tissue culture-treated 96 well plates (Corning-3699) at a density of 30,000 cell/well, 20,000 cell/well, 30,000 cell/well for NCI-H358, AGS and SW620 respectively, and allowed for attachment overnight. Diluted compounds were then added in a final concentration of 0.5% DMSO. After 4 hours of incubation, the medium was removed, 100 μL of 4% formaldehyde was added, and the assay plates were incubated at room temperature for 20 minutes. The plates were then washed once with phosphate buffered saline (PBS), and permeabilized with 100 μL of chilled methanol for 10 minutes. Non-specific antibody binding to the plates was blocked using 50 μL 1×BSA blocking buffer (Thermo-37520, 10-fold dilution by Phosphate-Buffered Saline Tween (PBST) for at least 1 hour at room temperature.

The amount of phosphor-ERK was determined using an antibody specific for phosphorylated form of ERK. Primary antibody (pERK, CST-4370, Cell Signaling Technology) was diluted 1:300 in blocking buffer, with 50 μL aliquoted to each well, and incubated overnight at 4° C. Cells were washed five times for 5 minutes with PBST. Secondary antibody (HRP-linked anti-rabbit IgG, CST-7074, Cell Signaling Technology) was diluted 1:1000 in blocking buffer, and 50 μL was added to each well and incubated 1-2 hours at room temperature. Cells were washed 5 times for 5 minutes with PBST, 100 μL TMB ELISA substrate (abcam-ab171523) were added and gently shake for 20 minutes. 50 μL stop solution (abcam-ab171529) were added, and then read the signal (OD450) by EnVision.

IC₅₀ was determined by fitting a 4-parameter sigmoidal concentration response model.

TABLE 4
Activity of Examples and Compounds of present
invention in KRAS pERK inhibition assay

| Example | G12C IC₅₀ (μM) | G12D IC₅₀ (μM) | G12V IC₅₀ (μM) |
|---|---|---|---|
| RM018 | 0.008 | >10 | >1 |
| RM461 | 0.007 | >10 | >10 |
| RM46-A | 0.049 | >10 | 7.022 |
| RM351 | 0.356 | >10 | >10 |
| Example 1 | 0.008 | 0.469 | / |
| Example 2 | 0.003 | 0.056 | 0.037 |
| Example 3 | 0.009 | 0.683 | 0.245 |
| Example 4 | 0.042 | 0.297 | 0.051 |
| Example 5 | 0.008 | 0.105 | 0.026 |
| Example 7 | 0.513 | 0.236 | / |
| Example 8 | 0.436 | / | / |
| Example 9 | 0.229 | / | 0.742 |
| Example 10 | 0.003 | 0.031 | 0.011 |
| Example 11 | 0.083 | 0.164 | 0.380 |
| Example 12 | 0.006 | 0.010 | 0.012 |
| Example 13 | 0.189 | / | 0.274 |
| Example 14 | 0.063 | 0.529 | 0.106 |
| Example 15 | 0.032 | 0.291 | 0.067 |
| Example 16 | 0.043 | 0.528 | 0.078 |
| Example 17 | 0.023 | / | 0.124 |
| Example 18 | 0.003 | 0.026 | 0.028 |
| Example 19 | 0.003 | 0.028 | 0.008 |
| Example 20 | 0.001 | 0.011 | 0.003 |
| Example 21 | 0.001 | 0.014 | 0.005 |
| Example 22 | 0.055 | 0.199 | 0.102 |
| Example 23 | 0.002 | 0.018 | 0.008 |
| Example 24 | 0.002 | 0.065 | 0.042 |
| Example 25 | 0.001 | 0.013 | 0.003 |
| Example 26 | 0.001 | 0.035 | 0.013 |

### Example 31

### Cell Viability Assay

The purpose of this cellular assay was to determine the effects of test compounds on the proliferation of human cancer cell lines NCI-H358 (ATCC-CRL5807) cells, AGS (ATCC-CRL-1739) cells, SW620 (ATCC-CCL-227) over a 3-day treatment period by quantifying the amount of NADPH present at endpoint using Cell Counting Kit-8.

Cells were seeded at 5,000 cells/well (NCI-H358), 2,000 cells/well (AGS) 2,000 cells/well (SW620) in 96-well assay plates (Corning-3699) and incubated overnight. On the day of the assay, diluted compounds were then added in a final concentration of 0.5% DMSO. After 72 hours incubation, a tenth of the volume of cell counting kit 8(Dnjindo-CK04) was added into each well. Read the signal (OD450 minus OD650) using EnVision after 2 hours incubation. IC₅₀ was determined by fitting a 4-parameter sigmoidal concentration response model.

TABLE 5
Activity of Examples and Compounds of present
invention in KRAS Cell viability assay

| Example | G12C IC50 (μM) | G12D IC50 (μM) | G12V IC50 (μM) |
|---|---|---|---|
| RM018 | 0.004 | >10 | >1 |
| RM461 | 0.022 | 2.708 | 4.045 |
| RM461-A | 0.045 | 7.916 | 6.875 |
| RM351 | 1.393 | 2.104 | 4.807 |
| Example 1 | 0.004 | / | / |
| Example 2 | 0.002 | 0.083 | 0.156 |
| Example 3 | 0.041 | 1.298 | 0.684 |
| Example 4 | 0.030 | / | 0.162 |
| Example 5 | 0.077 | 0.226 | 0.057 |
| Example 6 | 0.797 | / | / |
| Example 9 | 0.029 | / | 0.853 |
| Example 10 | 0.0004 | 0.052 | 0.059 |
| Example 11 | 0.118 | / | 0.161 |
| Example 12 | 0.005 | 0.016 | 0.016 |
| Example 13 | 0.066 | / | 0.328 |
| Example 14 | 0.142 | 1.384 | 0.367 |
| Example 15 | 0.089 | 1.241 | 0.281 |
| Example 16 | 0.077 | 1.166 | 0.294 |
| Example 17 | 0.006 | 1.133 | 0.449 |
| Example 18 | 0.0007 | 0.019 | 0.026 |
| Example 19 | 0.0009 | 0.018 | 0.015 |
| Example 20 | 0.0002 | 0.018 | 0.005 |
| Example 21 | 0.0003 | 0.044 | 0.008 |
| Example 22 | 0.0163 | 0.868 | 0.255 |
| Example 23 | <0.0006 | 3.410 | 0.015 |
| Example 24 | <0.0006 | 0.886 | 0.489 |
| Example 25 | <0.0006 | 0.094 | 0.006 |
| Example 26 | <0.0006 | 0.140 | 0.045 |

### Example 32

### Human Hepatocytes Stability Assay

The hepatocyte stability assay measures the rate of disappearance of a compound from incubations with cryopreserved suspension hepatocytes from human. Positive controls, including Midazolam, Raloxifene and Dextromethorphan, are included in every experiment. Incubations consist of 1 μM tested compound and suspension of human hepatocytes (1×10⁶ cells/mL) in supplemented Williams' E Medium with 1000 FBS and 0.5% Penicillin-streptomycin. The hepatocyte suspension was incubated with intermittent shaking 900 rpm at 37° C., in a 5% CO₂ incubator. The reaction was stopped by adding methanol containing internal standard (2 μM Tolbutamide) at 2, 10, 20, 40, 60 and 120 minutes after compound addition, depletion of the parent compound was monitored by LC-MS/MS analysis. For human data, CL_hep (mL/min/kg)>16.24 is high clearance, CL_hep (mL/min/kg)<6.96 is low clearance. 16.24<CL_hep (mL/min/kg)>6.96 is medium clearance.

TABLE 6
Human hepatocytes stability of Examples
and Compounds of present invention

| | CL_hep (Human) | Clearance |
|---|---|---|
| Example | (ml/min/kg) | Category |
| RM461 | 16.4 | high |
| RM351 | 15.5 | medium to high |
| RM461-A | 5.5 | low |
| Example 10 | <2.4 | low |
| Example 19 | 10.1 | medium |
| Example 21 | <2.4 | low |
| Example 23 | 7.9 | medium |
| Example 25 | 9.8 | medium |

Above result clearly shows that reference compounds (RM461 and RM351) showed high clearance while compounds of current invention maintained the low and moderate clearance in human hepatocytes stability assay. Particularly, comparing reference compound RM461 and its analogue RM461-A, the only difference is that the chloroacetamide was replaced by 2-chloro-2-fluoro-acetamide like the compounds of this invention, surprisingly such small change could increase the human hepatocyte stability compared with reference compounds.

1. A compound of formula (I), 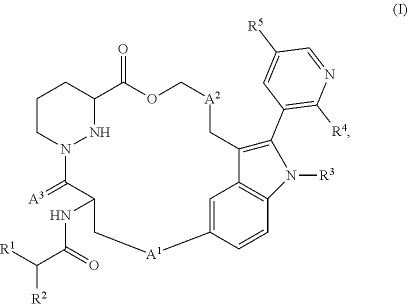
wherein
R¹ is 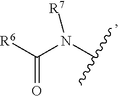
1-oxo-2,7-diazaspiro[4.4]nonan-2-yl substituted by (dihaloC₁₋₆ alkyl)carbonyl or 6-oxo-2,7-diazaspiro[4.5]decan-7-yl substituted by (dihaloC₁₋₆alkyl)carbonyl;
wherein R⁶ is azetidinyl substituted by C₂₋₆alkynyl,
cycloalkyl substituted by formyl, C₂₋₆alkynyl, pyridinylC₂₋₆alkynyl or [(C₁₋₆alkyl)₂(oxo)-1⁶-sulfanylidene]C₁₋₆alkylcarbonyl,
piperidinyl once or twice substituted by substituents independently selected from halogen, (dihaloC₁₋₆alkyl)carbonyl and C₂₋₆alkynyl, or
pyrrolidinyl substituted by (C₁₋₆alkylcarbonyl)carbonyl, (dihaloC₁₋₆alkyl)carbonyl, C₂₋₆alkynyl, cyanoC₁₋₆alkyl, cycloalkylcarbonyl, or triazolylC₂₋₆alkenylcarbonyl; and
R⁷ is C₁₋₆alkyl;
R² is C₁₋₆alkyl;
R³ is C₁₋₆alkyl or haloC₁₋₆alkyl;
R⁴ is C₁₋₆alkoxyC₁₋₆alkyl;
R⁵ is H, morpholinyl, (haloC₁₋₆alkyl)piperazinyl or C₁₋₆alkylpiperazinyl;
A¹ is thiazolylene or phenylene, said phenylene being substituted by hydroxy;
A² is C₁₋₆alkylene;
A³ is O;
or a pharmaceutically acceptable salt thereof.

2. A compound in accordance with claim 1, wherein the compound is a compound of formula (Ia), 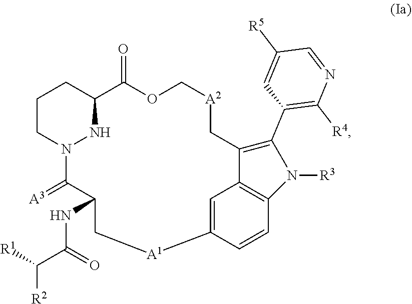
wherein
R¹ is 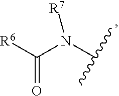
1-oxo-2,7-diazaspiro[4.4]nonan-2-yl substituted by (dihaloC₁₋₆alkyl)carbonyl or 6-oxo-2,7-diazaspiro[4.5]decan-7-yl substituted by (dihaloC₁₋₆alkyl)carbonyl;
wherein R⁶ is azetidinyl substituted by C₂₋₆alkynyl,
cycloalkyl substituted by formyl, C₂₋₆alkynyl, pyridinylC₂₋₆alkynyl or [(C₁₋₆alkyl)₂(oxo)-1⁶-sulfanylidene]C₁₋₆alkylcarbonyl,
piperidinyl once or twice substituted by substituents independently selected from halogen, (dihaloC₁₋₆alkyl)carbonyl and C₂₋₆alkynyl, or
pyrrolidinyl substituted by (C₁₋₆alkylcarbonyl)carbonyl, (dihaloC₁₋₆alkyl)carbonyl, C₂₋₆alkynyl, cyanoC₁₋₆alkyl, cycloalkylcarbonyl, or triazolylC₂₋₆alkenylcarbonyl; and
R⁷ is C₁₋₆alkyl;
R² is C₁₋₆alkyl;
R³ is C₁₋₆alkyl or haloC₁₋₆alkyl;
R⁴ is C₁₋₆alkoxyC₁₋₆alkyl;
R⁵ is H, morpholinyl, (haloC₁₋₆alkyl)piperazinyl or C₁₋₆alkylpiperazinyl;
A¹ is thiazolylene or phenylene, said phenylene being substituted by hydroxy;
A² is C₁₋₆alkylene;
A³ is O;
or a pharmaceutically acceptable salt thereof.

3. A compound according to claim 2, or a pharmaceutically acceptable salt thereof, wherein:
R¹ is 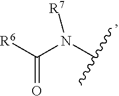
1-oxo-2,7-diazaspiro[4.4]nonan-2-yl substituted by (dihaloC₁₋₆ alkyl)carbonyl, or 6-oxo-2,7-diazaspiro[4.5]decan-7-yl substituted by (dihaloC₁₋₆alkyl)carbonyl;
wherein R⁶ is pyrrolidinyl substituted by (dihaloC₁₋₆alkyl)carbonyl; and
R⁷ is C₁₋₆alkyl.

4. A compound according to claim 2, or a pharmaceutically acceptable salt thereof, wherein:
R¹ is 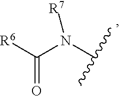
1-oxo-2,7-diazaspiro[4.4]nonan-2-yl substituted by chloro(fluoro)acetyl, or 6-oxo-2,7-diazaspiro[4.5]decan-7-yl substituted by chloro(fluoro)acetyl;
wherein R⁶ is pyrrolidinyl substituted by chloro(fluoro)acetyl; and
R⁷ is methyl.

5. A compound according to claim 2, or a pharmaceutically acceptable salt thereof wherein R¹ is

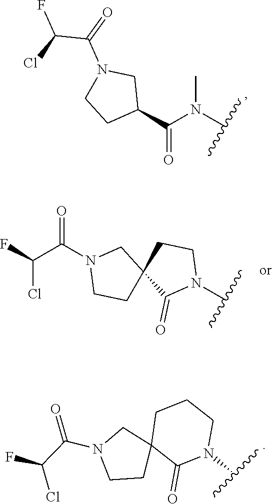

6. A compound according to claim 2, or a pharmaceutically acceptable salt thereof wherein R² is isopropyl.

7. A compound according to claim 2, or a pharmaceutically acceptable salt thereof wherein R³ is ethyl or trifluoroethyl.

8. A compound according to claim 2, or a pharmaceutically acceptable salt thereof wherein R⁴ is methoxyethyl.

9. A compound according to claim 2, or a pharmaceutically acceptable salt thereof wherein R⁴ is

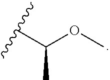 [SMILES: *[C@H](C)OC]

10. A compound according to claim 2, or a pharmaceutically acceptable salt thereof wherein R⁵ is morpholinyl or methylpiperazinyl.

11. A compound according to claim 2, or a pharmaceutically acceptable salt thereof, wherein A¹ is or 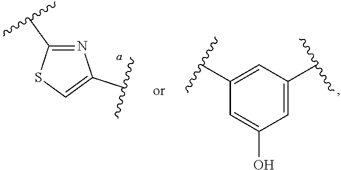 [SMILES: *c1csc(*)n1 ; *c1cc(*)cc(O)c1]
wherein bond “a” connects to indole ring.

12. A compound according to claim 2, or a pharmaceutically acceptable salt thereof, wherein A² is dimethylmethylene.

13. A compound according to claim 2, wherein
R¹ is 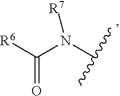
1-oxo-2,7-diazaspiro[4.4]nonan-2-yl substituted by (dihaloC₁₋₆alkyl)carbonyl or 6-oxo-2,7-diazaspiro[4.5]decan-7-yl substituted by (dihaloC₁₋₆alkyl)carbonyl;
wherein R⁶ is pyrrolidinyl substituted by (dihaloC₁₋₆alkyl)carbonyl; and
R⁷ is C₁₋₆alkyl;
R² is C₁₋₆alkyl;
R³ is C₁₋₆alkyl or haloC₁₋₆alkyl;
R⁴ is C₁₋₆alkoxyC₁₋₆alkyl;
R⁵ is morpholinyl or C₁₋₆alkylpiperazinyl;
A¹ is thiazolylene or phenylene, said phenylene being substituted by hydroxy;
A² is C₁₋₆alkylene;
A³ is O;
or a pharmaceutically acceptable salt thereof.

14. A compound according to claim 13, wherein
R¹ is or 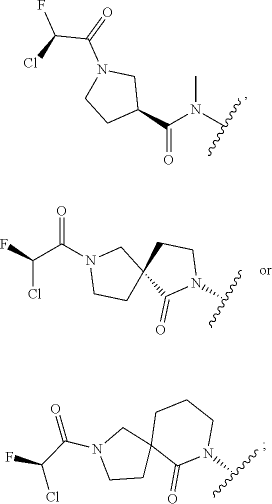
R² is isopropyl;
R³ is ethyl or trifluoroethyl;
R⁴ is 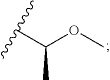 [SMILES: *[C@H](C)OC]
R⁵ is morpholinyl or methylpiperazinyl;
A¹ is 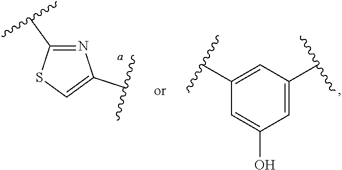 [SMILES: *c1csc(*)n1 ; *c1cc(*)cc(O)c1]
wherein bond “a” connects to indole ring;
A² is dimethylmethylene;
A³ is O;
or a pharmaceutically acceptable salt thereof.

15. A compound selected from:
(3S)-1-[(2S)-2-chloro-2-fluoro-acetyl]-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-pyrrolidine-3-carboxamide;
(3S)-1-[(2R)-2-chloro-2-fluoro-acetyl]-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-pyrrolidine-3-carboxamide;
N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-3-formyl-N-methyl-cyclobutanecarboxamide;
N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-3-ethynyl-N-methyl-cyclobutanecarboxamide;
(3S)-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-1-(2-oxopropanoyl)pyrrolidine-3-carboxamide;
(3S)-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-1-prop-2-ynyl-pyrrolidine-3-carboxamide;
(3S)-1-(cyanomethyl)-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-pyrrolidine-3-carboxamide;
3-[2-[dimethyl(oxo)-λ⁶-sulfanylidene]acetyl]-N-[(1S)-1-[[(8S,14S)-22-ethyl-4-hydroxy-(21″)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-cyclobutanecarboxamide;
(3S)-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-1-[(E)-3-(1,2,4-triazol-1-yl)prop-2-enoyl]pyrrolidine-3-carboxamide;
(3S)-1-[(2R)-2-chloro-2-fluoro-acetyl]-N-[(1S)-1-[[(7S,13S)-21-ethyl-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.12,5.19,13.022,26]octacosa-1(25),2,5(28),19,22(26),23-hexaen-7-yl]carbamoyl]-2-methyl-propyl]-N-methyl-pyrrolidine-3-carboxamide;
(3S)-1-(bicyclo[1.1.0]butane-1-carbonyl)-N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-pyrrolidine-3-carboxamide;
(3S)-1-[(2R)-2-chloro-2-fluoro-acetyl]-N-[(1S)-1-[[(8S,14S)-22-ethyl-4-hydroxy-(21M)-21-[2-[(1S)-1-methoxyethyl]-5-(4-methylpiperazin-1-yl)-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-pyrrolidine-3-carboxamide;
N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-N-methyl-3-[2-(2-pyridyl)ethynyl]cyclobutanecarboxamide;
N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-3-ethynyl-N-methyl-azetidine-1-carboxamide;
N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-4-ethynyl-N-methyl-piperidine-1-carboxamide;
N-[(1S)-1-[[(8S,14S)-22-ethyl-4-hydroxy-(21M)-21-[2-[(1S)-1-methoxyethyl]-3-pyridyl]-18,18-dimethyl-9,15-dioxo-16-oxa-10,22,28-triazapentacyclo[18.5.2.12,6.110,14.023,27]nonacosa-1(26),2,4,6(29),20,23(27),24-heptaen-8-yl]carbamoyl]-2-methyl-propyl]-4-ethynyl-4-fluoro-N-methyl-piperidine-1-carboxamide;
(3S)-1-[(2R)-2-chloro-2-fluoro-acetyl]-N-[(1S)-1-[[(7S,13S)-21-ethyl-(20M)-20-[2-[(1S)-1-methoxyethyl]-3-pyridyl]-17,17-dimethyl-8,14-dioxo-15-oxa-4-thia-9,21,27,28-tetrazapentacyclo[17.5.2.12,5.19,13.022,26]octacosa-1(25),2,5(28),19,22(26),23-hexaen-7-yl]carbamoyl]-2-methyl-propyl]-N-methyl-pyrrolidine-3-carboxamide;
(2S)-2-[2-[(2R)-2-chloro-2-fluoro-acetyl]-6-oxo-2,7-diazaspiro[4.5]decan-7-yl]-N-[(7S,13S)-21-ethyl-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.12,5.19,13.022,26]octacosa-1(25),2,5(28),19,22(26),23-hexaen-7-yl]-3-methyl-butanamide;
(2S)-2-[2-[(2R)-2-chloro-2-fluoro-acetyl]-6-oxo-2,7-diazaspiro[4.5]decan-7-yl]-N-[(7S,13S)-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.12,5.19,13.022,26]octacosa-1(25),2,5(28),19,22(26),23-hexaen-7-yl]-3-methyl-butanamide;
1-[(2R)-2-chloro-2-fluoro-acetyl]-4-fluoro-N-[(1S)-1-[[(7S,13S)-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.12,5.19,13.022,26]octacosa-1(25),2,5(28),19,22(26),23-hexaen-7-yl]carbamoyl]-2-methyl-propyl]-N-methyl-piperidine-4-carboxamide;
(3S)-1-[(2R)-2-chloro-2-fluoro-acetyl]-N-[(1S)-1-[[(7S,13S)-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.12,5.19,13.022,26]octacosa-1(25),2,5(28),19,22(26),23-hexaen-7-yl]carbamoyl]-2-methyl-propyl]-N-methyl-pyrrolidine-3-carboxamide;
(3S)-1-[(2R)-2-chloro-2-fluoro-acetyl]-N-[(1S)-1-[[(7S,13S)-(20M)-20-[2-[(1S)-1-methoxyethyl]-3-pyridyl]-17,17-dimethyl-8,14-dioxo-21-(2,2,2-trifluoroethyl)-15-oxa-4-thia-9,21,27,28-tetrazapentacyclo[17.5.2.12,5.19,13.022,26]octacosa-1(25),2,5(28),19,22(26),23-hexaen-7-yl]carbamoyl]-2-methyl-propyl]-N-methyl-pyrrolidine-3-carboxamide;
(2S)-2-[(5S)-7-[(2R)-2-chloro-2-fluoro-acetyl]-1-oxo-2,7-diazaspiro[4.4]nonan-2-yl]-N-[(7S,13S)-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.12,5.19,13.022,26]octacosa-1(25),2,5(28),19,22(26),23-hexaen-7-yl]-3-methyl-butanamide;
(2S)-2-[(5R)-7-[(2R)-2-chloro-2-fluoro-acetyl]-1-oxo-2,7-diazaspiro[4.4]nonan-2-yl]-N-[(7S,13S)-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.12,5.19,13.022,26]octacosa-1(25),2,5(28),19,22(26),23-hexaen-7-yl]-3-methyl-butanamide;
(3S)-1-[(2R)-2-chloro-2-fluoro-acetyl]-N-[(1S)-1-[[(7S,13S)-(20M)-20-[2-[(1S)-1-methoxyethyl]-5-morpholino-3-pyridyl]-17,17-dimethyl-8,14-dioxo-21-(2,2,2-trifluoroethyl)-15-oxa-4-thia-9,21,27,28-tetrazapentacyclo[17.5.2.12,5.19,13.022,26]octacosa-1(25),2,5(28),19,22(26),23-hexaen-7-yl]carbamoyl]-2-methyl-propyl]-N-methyl-pyrrolidine-3-carboxamide; and
(3S)-1-[(2R)-2-chloro-2-fluoro-acetyl]-N-[(1S)-1-[[(7S,13S)-21-ethyl-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.12,5.19,13.022,26]octacosa-1(25),2,5(28),19,22(26),23-hexaen-7-yl]carbamoyl]-2-methyl-propyl]-N-methyl-pyrrolidine-3-carboxamide;
or a pharmaceutically acceptable salt thereof.

16. Compound RM461-A with structure of 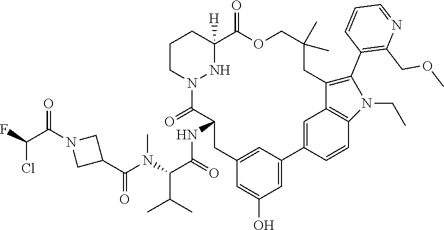 [SMILES: CCn1c(-c2cccnc2COC)c2c3cc(ccc31)-c1cc(O)cc(c1)C[C@H](NC(=O)[C@H](C(C)C)N(C)C(=O)C1CN(C(=O)[C@H](F)Cl)C1)C(=O)N1CCC[C@H](N1)C(=O)OCC(C)(C)C2]
or a pharmaceutically acceptable salt thereof.

17. A process for the preparation of a compound according to claim 2, or a pharmaceutically acceptable salt thereof, comprising any of the following steps:
a) a coupling reaction between compound of formula (IX), 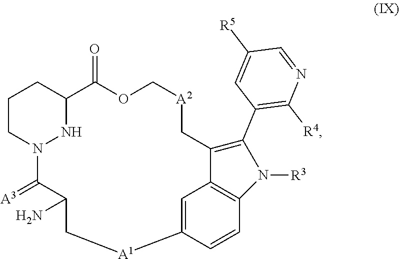
and acid (X), 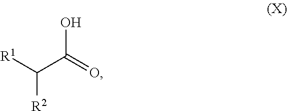
with coupling reagent in the presence of a base;
b) a coupling reaction between compound of formula (XIII), 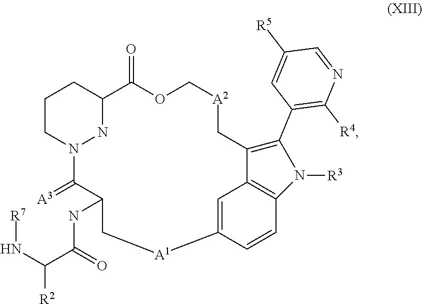
and acid (XIV), 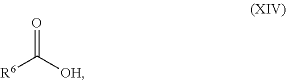
with coupling reagent in the presence of a base; or
c) a coupling reaction between compound of formula (XVIII), 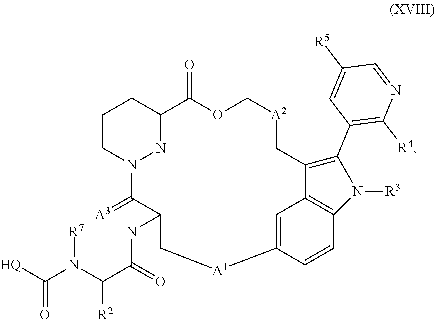
and acid (XIX), 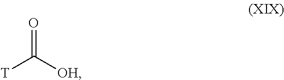
with coupling reagent in the presence of a base;
wherein Q is heterocyclylene; T is (C₁₋₆alkyl)₂oxooxetanyl, C₁₋₆alkylcarbonyl, dihaloC₁₋₆alkyl, oxooxetanylamino, C₂₋₆alkynyl, C₃₋₈alkadienyl, cyanoC₁₋₆alkyl, cycloalkyl, morpholinylC₂₋₆alkynyl, oxoazetidinyl, pyridinylC₂₋₆alkynyl or triazolylC₂₋₆alkenyl; R¹, R², R³, R⁴, R⁵, R⁶, R⁷, A¹, A² and A³ are defined as in claim 2; the coupling reagent is T₃P, HATU, PyBOP or EDCI/HOBt; and the base is TEA, DIEPA or DMAP.

18. (canceled)

19. A pharmaceutical composition comprising a compound in accordance with claim 2, or a pharmaceutically acceptable salt thereof, and a therapeutically inert carrier.

20-27. (canceled)

28. A method for the treatment of a KRAS mutation driven cancer in a human in need thereof, comprising administering a therapeutically effective amount of a compound of claim 2, or a pharmaceutically acceptable salt thereof, to the human; wherein the cancer is selected from pancreatic adenocarcinoma, colorectal cancer, and non-small cell lung cancer.

29-30. (canceled)

31. A method for the treatment of a KRAS mutation driven cancer in a human in need thereof, comprising administering a therapeutically effective amount of a compound of claim 15, or a pharmaceutically acceptable salt thereof, to the human; wherein the cancer is selected from pancreatic adenocarcinoma, colorectal cancer, and non-small cell lung cancer.

32. A method for the treatment of a KRAS G12C protein-related disease in a human in need thereof, comprising administering a therapeutically effective amount of a compound of claim 2, or a pharmaceutically acceptable salt thereof, to the human.

33. A method of treating a KRAS G12C, G12D and G12V protein-related disease in a human in need thereof, comprising administering a therapeutically effective amount of a compound of claim 2, or a pharmaceutically acceptable salt thereof, to the human.

34. A pharmaceutical composition comprising a compound in accordance with claim 15, or a pharmaceutically acceptable salt thereof, and a therapeutically inert carrier.

35. A pharmaceutical composition comprising a compound in accordance with claim 16, or a pharmaceutically acceptable salt thereof, and a therapeutically inert carrier.
